(19) United States
(12) Patent Application Publication
(10) Pub. No.: US 2026/0152501 A1
(43) Pub. Date: Jun. 4, 2026

(54) CYCLIN MODULATOR

(71) Applicant: EUBULUS BIOTHERAPEUTICS (HONG KONG) LIMITED, HONG KONG (HK)

(72) Inventors: Sheldon CAO, Jiaxing, Zhejiang (CN); Xiaolei WANG, Jiaxing, Zhejiang (CN); Feng DING

(21) Appl. No.: 19/106,717

(22) PCT Filed: Aug. 28, 2023
(86) PCT No.: PCT/CN2023/115312

§ 371 Date: Feb. 26, 2025

(30) Foreign Application Priority Data
Aug. 26, 2022 (CN) 202211037727.8

(51) Int. Cl. C07D 473/16; A61K 31/52; A61K 31/5377; A61K 47/54; A61K 47/64; A61P 35/00; C07D 473/18

(52) U.S. Cl. CPC C07D 473/16; A61K 31/52; A61K 31/5377; A61K 47/555; A61K 47/64; A61P 35/00; C07D 473/18

(57) ABSTRACT

A cyclin modulator, specifically a compound of formula (I) or a pharmaceutically acceptable salt thereof are provided.

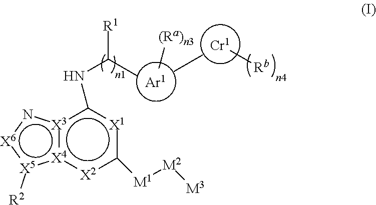

### TECHNICAL FIELD

The present invention belongs to the field of pharmaceutical chemistry, specifically, relates to a cyclin modulator.

### BACKGROUND

Unlike the action of most traditional drugs that directly inhibit molecular targets, molecular glue degraders kill cancer cells by destroying target proteins via the ubiquitin-proteasome system. For example, the multiple myeloma drug lenalidomide is a molecular glue degrader that recruits E3 ubiquitin ligase to mark the target protein in the cell and then degrade it. Unlike traditional small molecule inhibitors, molecular glue drives target ubiquitination and induces degradation in a catalytic manner, which is a new strategy that can inactivate targets that are difficult to treat with traditional pharmacological methods. Molecular glues also cleverly avoid the limitations of traditional inhibitors, turning some targets from “non-drugable” to “drugable”.

Cell cycle is a basic process of cell life activities, which controls the transition of cells from the quiescent phase to the growth and proliferation phase. Cyclin-dependent kinases (CDKs) and Cyclins are the core molecules in the entire cell cycle regulation mechanism. In normal cells, the activity of cyclins is strictly controlled by their cell cycle-specific transcription and protein degradation, and affected by some CDK inhibitory proteins. In addition to promoting cell division, cyclins are also responsible for regulating various cell functions. This mechanism is jointly operated by cyclins and their catalytic partners, cyclin-dependent kinases (CDKs). However, these influencing factors are often out of control in human cancers, leading to abnormal activation of cyclins Abnormal activity of the cell cycle mechanism is present in almost all types of tumors and is the driving force behind tumorigenesis. Targeting a specific cyclin may be an effective anti-cancer strategy.

So far, there are few CDK inhibitors on the market, and all of them are CDK4/6 inhibitors.

Inhibitors that selectively target other CDK families are difficult to achieve the required specificity. Almost all of them are multi-target inhibitors, which exhibit unacceptable toxicity in clinical practice, thus restricting their clinical application. In addition, although kinase inhibitors can lead to the removal of kinase subunits, they also maintain the integrity of cyclins, which may trigger compensatory mechanisms. The degradation of kinases may lead to effects that last longer than their inhibitory effects.

Although molecular glue degraders are very ideal, have good clinical effects and are very popular, there are still only a few molecular glue degraders discovered so far, and most of them are discovered by chance. As a newly discovered CyclinK degrader, CR8 is firstly a multi-kinase inhibitor that can inhibit the activity of multiple cyclins in the CDK family, and the resulting toxicity limits its clinical application. In addition to poor selectivity, its activity in degrading CyclinK is also average, which limits its use in catalytic amounts.

In summary, there is an urgent need in the art to develop a class of cyclin modulator such as Cyclin degraders with higher activity and/or lower toxicity.

### SUMMARY OF THE INVENTION

The purpose of the present invention is to provide a cyclin degrader with higher activity, or a novel cyclin modulator.

In the first aspect of the present invention, provided is a compound of formula (I), or a pharmaceutically acceptable salt thereof,

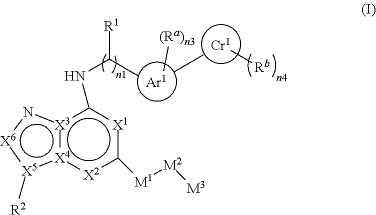

wherein,
R¹ is each independently H or C₁₋₄alkyl;
subscript n1 is 1, 2 or 3;
Ring Ar¹ is selected from the group consisting of: C₆₋₁₀ aromatic ring, 5 to 10 membered heteroaromatic ring, and 5 to 10 membered bridged ring;
Ring Cr¹ is selected from the group consisting of: H, C₃₋₁₀ carbocyclyl, 3 to 10 membered heterocyclyl, C₆₋₁₀ aryl, and 5 to 10 membered heteroaryl;
R^{a} and R^{b} are each independently selected from the group consisting of: H, R^{e} and R; or R^{a} and R^{b} together with Ring Ar¹ and Ring Cr¹ form

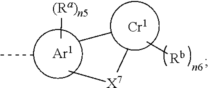

wherein,
X⁷ is each independently selected from the group consisting of: —O—, —S—, —N(R^{c})—, —C(R^{c})₂—, and —C(R^{c})₂—C(R^{c})₂—;
subscripts n5 and n6 are each independently 0, 1, 2 or 3;
R^{e} is each independently selected from the group consisting of: hydroxyl, C₁₋₆alkyl, —O—C₁₋₆alkyl, and —O—C₁₋₆alkylene-Rf;
wherein, R^{f} is selected from the group consisting of: —CN, —OH, —NH₂, —NH(C₁₋₆alkyl), and —N(C₁₋₆alkyl)₂;
subscripts n3 and n4 are each independently 0, 1, 2, 3 or 4;
R² is selected from the group consisting of: H, CN, optionally substituted C₁₋₆alkyl, optionally substituted C₂₋₆ alkenyl, optionally substituted C₂₋₆ alkynyl, optionally substituted C₃₋₈ cycloalkyl, and optionally substituted 3 to 8 membered heterocyclyl;
X¹, X² and X⁶ are each independently N or C(R^{c});
X³, X⁴ and X⁵ are each independently N or C;
M¹ is selected from the group consisting of: null, X⁸, and (M⁴)ₛ; wherein, X⁸ is N(R^{c}) or C(R^{c})₂;
M⁴ is each independently selected from the group consisting of: O, S, C(O)O, C(O), N(R^{c}) and C₁₋₄ alkylene;
s is 1, 2 or 3;
M² is null or a ring of formula A;

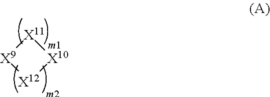

in formula A, X⁹ is the position attached to M¹, X¹⁰ is the position attached to M³; X⁹ is N or C(R^{m}), X¹⁰ is selected from the group consisting of: O, S, N and C(R^{m}); X¹¹ and X¹² are each independently selected from the group consisting of: —C(R^{m})₂—, and —N(R^{m})—; subscripts m1 and m2 are each independently 0, 1, 2 or 3, and m1+m2≥2;
wherein, R^{m} is each independently R^{e} or R^{m1}; wherein,
R^{m1} is each independently selected from the group consisting of: hydroxyl, optionally substituted C₁₋₆ alkyl, optionally substituted C₁₋₆hydroxyalkyl, and optionally substituted C₁₋₆ haloalkyl; or, two R^{m1} together form a single bond, optionally substituted C₁₋₄alkylene or optionally substituted 1 to 4 membered heteroalkylene;
M³ is selected from the group consisting of: null, R³, and —NH—R³; wherein,
R³ is selected from the group consisting of: H, optionally substituted C₁₋₆alkyl, optionally substituted C₁₋₆-hydroxyalkyl, and optionally substituted C₁₋₆-haloalkyl;
R^{c} is each independently H or C₁₋₄alkyl;
unless specifically defined, said optionally substituted means unsubstituted or one or more (such 1, 2, or 4) hydrogen atoms in the group are substituted with substituent R, and R is selected from the group consisting of: D, halogen, C₁₋₆ alkyl, C₁₋₆ haloalkyl, C₁₋₆ hydroxyalkyl, C₂₋₆ alkenyl, C₂₋₆ alkynyl, —CN, —OR′, —NO₂, —NR′R″, —SR′, —OC(O)R′, —C(O)R′, —CO₂R′, —CONR′, —OC(O)NR′R″, —NR″C(O)R′, —NR″—C(O)NR′R″, —NR″C(O)₂R′, —S(O)R′, —S(O)₂R′, —S(O)₂NR′R″, —NR″S(O)₂R′, C₃₋₁₀cycloalkyl optionally substituted with one or more R′″, 4 to 10 membered heterocycloalkyl optionally substituted with one or more R′″, C₆₋₁₀ aryl optionally substituted with one or more R′″, 5 to 10 membered heteroaryl optionally substituted with one or more R′″, —C₁₋₄alkylene-C₃₋₁₀ cycloalkyl optionally substituted with one or more R′″, —C₁₋₄alkylene-4 to 10 membered heterocycloalkyl optionally substituted with one or more R′″, —C₁₋₄alkylene-C₆₋₁₀aryl optionally substituted with one or more R′″, —C₁₋₄alkylene-5 to 10 membered heteroaryl optionally substituted with one or more R′″;
each R′ is independently H, D, a group selected from the following group that is optionally substituted with one or more R′″: C₁₋₆alkyl, C₃₋₁₀ cycloalkyl, 4 to 10 membered heterocycloalkyl, C₆₋₁₀aryl, 5 to 10 membered heteroaryl, —C₁₋₄alkylene-C₃₋₁₀cycloalkyl, —C₁₋₄alkylene-4 to 10 membered heterocycloalkyl, —C₁₋₄alkylene-C₆₋₁₀aryl and —C₁₋₄alkylene-5 to 10 membered heteroaryl;
each R″ is selected from the group consisting of: H, D, C₁₋₄alkyl, C₁₋₄haloalkyl, and C₃₋₄ cycloalkyl;
each R′″ is independently selected from the group consisting of: D, halogen, hydroxyl, nitro, CN, C₁₋₆alkyl, and C₁₋₆ haloalkyl.

In another preferred embodiment, the compound is not CR8 as shown below:

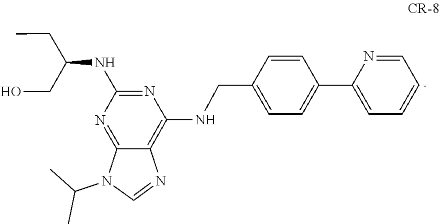 [SMILES: CC[C@H](CO)Nc1nc(NCc2ccc(-c3ccccn3)cc2)c2ncn(C(C)C)c2n1]

In another preferred embodiment, R¹ is H. In another preferred embodiment, n1=1. In another preferred embodiment, R¹ is H, and n1=1.

In another preferred embodiment, Ring Ar¹ is selected from the group consisting of: C₆₋₁₀ aromatic ring, and 5 to 10 membered heteroaromatic ring.

In another preferred embodiment, Ring Ar¹ is selected from the group consisting of: benzene ring, and 5 to 10 membered heteroaromatic ring.

In another preferred embodiment, Ring Ar¹ is selected from the group consisting of:

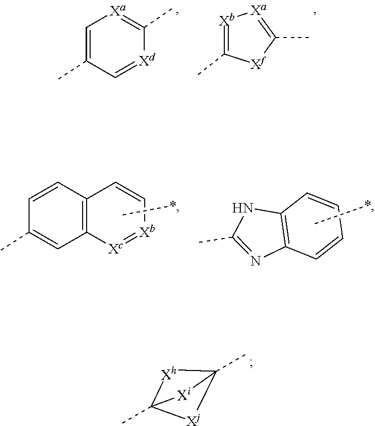

wherein, * represents the position attached to Ring Cr¹; X^{a}, X^{b}, X^{c} and X^{d} are each independently CH or N; X^{g} is selected from the group consisting of: NH, O, and S; X^{h}, Xⁱ and X^{j} are each independently —CH₂— or —CH₂—CH₂—.

In another preferred embodiment,

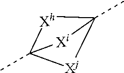

is

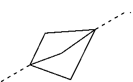

In another preferred embodiment, in Ring Ar¹,

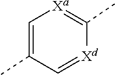

is

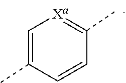

In another preferred embodiment, Ring Ar¹ is selected from the group consisting of:

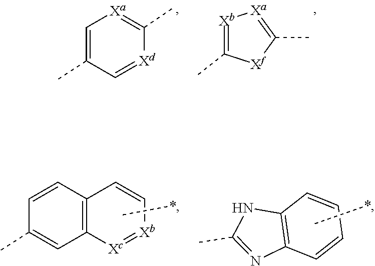

wherein, * represents the position attached to Ring Cr¹; X^{a}, X^{b}, X^{c} and X^{d} are each independently CH or N; X^{g} is selected from the group consisting of: NH, O, and S.

In another preferred embodiment, Ring Ar¹ is

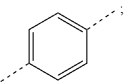

wherein, * represents the position attached to Ring Cr¹.

In another preferred embodiment,

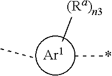

is

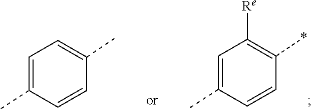

wherein * represents the position attached to Ring Cr¹

In another preferred embodiment, in Ring Cr¹, the carbocycle is saturated carbocycle or unsaturated carbocycle containing 1 or 2 double bonds.

In another preferred embodiment, in Ring Cr¹, the C₃₋₁₀ carbocycle is C₄₋₁₀ carbocycle; preferably, is C₄₋₆ carbocycle.

In another preferred embodiment, in Ring Cr¹, the 3 to 10 membered heterocyclyl is saturated 3 to 10 membered heterocyclyl.

In another preferred embodiment, in Ring Cr¹, the 3 to 10 membered heterocyclyl is 4 to 10 membered heterocyclyl; preferably, is 4 to 6 membered heterocyclyl.

In another preferred embodiment, Ring Cr¹ is selected from the group consisting of:

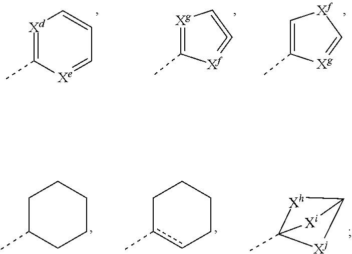

wherein, X^{d} and X^{e} are each independently N or CH; X^{f} is NH, S, or O; X^{g} is N or CH.

In another preferred embodiment,

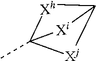

is

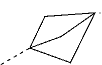

In another preferred embodiment, Ring Cr¹ is Ring Ar²; and Ring Ar² is selected from the group consisting of: C₆₋₁₀ aryl, and 5 to 10 membered heteroaryl.

In another preferred embodiment, Ring Ar² is selected from the group consisting of:

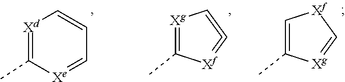

wherein, X^{d} and Xe are each independently —N— or —CH—; X is —NH—, —S—, or —O—; X^{g} is N or CH.

In another preferred embodiment, Ring Ar² is

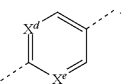

In another preferred embodiment, Ring Ar² is phenyl.

In another preferred embodiment, when M² is null, Ring Ar² is not a nitrogen-containing heteroaryl (wherein, the nitrogen-containing heteroaryl is a heteroaryl having 1 or 2 nitrogen heteroatoms and no other heteroatoms on the ring, such as pyridyl, pyrazolyl, imidazolyl and pyrazinyl).

In another preferred embodiment, when M2 is null, Ring Ar² is not

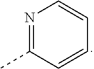

In another preferred embodiment, n3 is 0 (i.e., Ring Ar¹ is unsubstituted); or, n3 is 1, 2, 3 or 4 (i.e., Ring Ar¹ is substituted with 1, 2, 3 or 4 R^{a}), and R^{a} is each independently selected from the group consisting of: D, halogen, C₁₋₆alkyl, and C₁₋₆ haloalkyl.

In another preferred embodiment, n3 is 0. In another preferred embodiment, n3 is 1, and R^{a} is R^{c}.

In another preferred embodiment, n4 is 0 (i.e., Ring Cr¹ is unsubstituted); or, n4 is 1, 2, 3 or 4 (i.e., Ring Cr¹ is substituted with 1, 2, 3 or 4 R^{b}), and R^{b} is each independently selected from the group consisting of: D, halogen, C₁₋₆alkyl, and C₁₋₆ haloalkyl.

In another preferred embodiment, n4 is 0. In another preferred embodiment, n4 is 1, and Rb is R^{c}.

In another preferred embodiment, n3 is 0 (i.e., Ring Ar¹ is unsubstituted); or, n3 is 1, 2, 3 or 4 (i.e., Ring Ar¹ is substituted with 1, 2, 3 or 4 R^{a}), and R^{a} is each independently selected from the group consisting of: D, halogen, C₁₋₆alkyl, and C₁₋₆ haloalkyl.

In another preferred embodiment, when M2 is null,

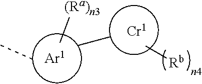

is not

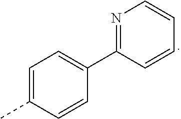

In another preferred embodiment,

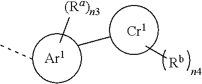

is

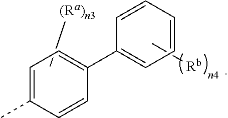

In another preferred embodiment, X¹ is N, X² is CR^{c}, X³ is C, X⁴ is C, X⁵ is N and X⁶ is CR^{c}. In another preferred embodiment, X¹ is CR^{c}, X² is CR^{c}, X³ is CR^{c}, X⁴ is N, X⁵ is C and X⁶ is CR^{c}. In another preferred embodiment, X¹ is N, X² is CR^{c}, X³ is CR^{c}, X⁴ is N, X⁵ is C and X⁶ is CR^{c}. In another preferred embodiment, X¹ is CR^{c}, X² is CR^{c}, X³ is CR^{c}, X⁴ is N, X⁵ is N and X⁶ is CR^{c}. In another preferred embodiment, X¹ is CR^{c}, X² is CR^{c}, X³ is N, X⁴ is CR^{c}, X⁵ is C and X⁶ is CR^{c}. In another preferred embodiment, X¹ is CR^{c}, X² is N, X³ is N, X⁴ is CR^{c}, X⁵ is C and X⁶ is CR^{c}. In another preferred embodiment, X¹ is N, X² is CR^{c}, X³ is N, X⁴ is CR^{c}, X⁵ is C and X⁶ is CR^{c}.

In another preferred embodiment, all R^{e} are H.

In another preferred embodiment, X¹ is N, X² is CH, X³ is C, X⁴ is C, X⁵ is N and X⁶ is CH. In another preferred embodiment, X¹ is CH, X² is CH, X³ is CH, X⁴ is N, X⁵ is C and X⁶ is CH. In another preferred embodiment, X¹ is N, X² is CH, X³ is CH, X⁴ is N, X⁵ is C and X⁶ is CH. In another preferred embodiment, X¹ is CH, X² is CH, X³ is CH, X⁴ is N, X⁵ is N and X⁶ is CH. In another preferred embodiment, X¹ is CH, X² is CH, X³ is N, X⁴ is CH, X⁵ is C and X⁶ is CH. In another preferred embodiment, X¹ is CH, X² is N, X³ is N, X⁴ is CH, X⁵ is C and X⁶ is CH. In another preferred embodiment, X¹ is N, X² is CH, X³ is N, X⁴ is CH, X⁵ is C and X⁶ is CH.

In another preferred embodiment,

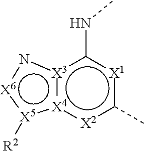

is selected from the group consisting of:

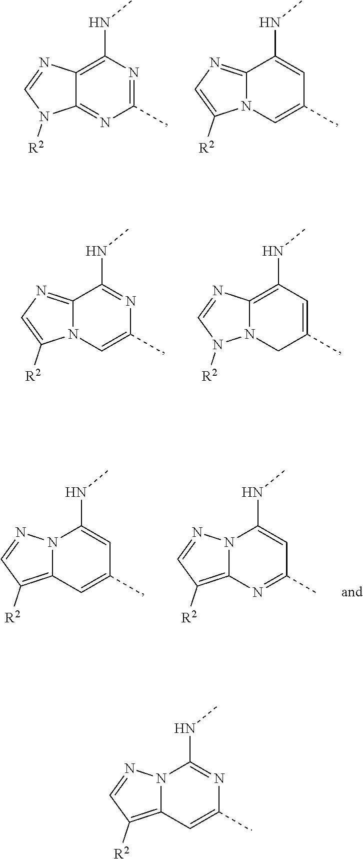

In another preferred embodiment,

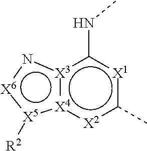

is

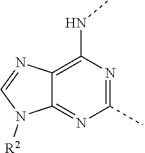

In another preferred embodiment, R² is optionally substituted C₁₋₆alkyl. In another preferred embodiment, R² is C₁₋₆alkyl. In another preferred embodiment, R² is selected from the group consisting of: methyl, ethyl, n-propyl, isopropyl, n-butyl, isobutyl, and tert-butyl.

In another preferred embodiment, in formula A, when two R^{m1} are located on the same carbon atom, the two R^{m1} together form optionally substituted C₂₋₄ alkylene or optionally substituted 2 to 4-membered heteroalkylene (i.e., the ring of formula A is a spiro ring).

In another preferred embodiment, in formula A, when two R^{m1} are located on two adjacent ring atoms, the two R^{m1} together form optionally substituted C₁₋₄ alkylene or optionally substituted 1 to 4-membered heteroalkylene (i.e., the ring of formula A is a fused ring).

In another preferred embodiment, in formula A, when two R^{m1} are located on two ring atoms separated by at least one ring atom, the two R^{m1} together form a single bond, optionally substituted C₁₋₃ alkylene or optionally substituted 1 to 3-membered heteroalkylene (i.e., the ring of formula A is a fused ring).

In another preferred embodiment, two R^{m1} together form C₁₋₄ alkylene.

In another preferred embodiment, 2≤m1+m2≤4 (i.e., a ring of formula A is a 4 to 6 membered ring).

In another preferred embodiment, when X¹⁰ is O or S, M³ is null; when X¹⁰ is N or C(R^{m}), M³ is R³⁻⁻ or —NH—R³.

In another preferred embodiment, M¹ is null or X⁸.

In another preferred embodiment, M¹ is null, M³ is —NH—R³, and M² is null.

In another preferred embodiment, R³ is optionally substituted C₁₋₆-hydroxyalkyl.

In another preferred embodiment,

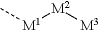

is

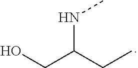

In another preferred embodiment, M¹ is null, M³ is null or R³, and M² is a ring of formula A.

In another preferred embodiment, m1 and m2 are each independently 1 or 2. In another preferred embodiment, both of m1 and m2 are 2.

In another preferred embodiment, X⁹ is N.

In another preferred embodiment, R³ is H.

In another preferred embodiment, X¹⁰ is N, M³ is R³, and R³ is H or C₁₋₆alkyl. In another preferred embodiment, X¹⁰ is N, M³ is R³, and R³ is H.

In another preferred embodiment, X¹¹ and X¹² are each independently —C(R^{m})₂—.

In another preferred embodiment, in X¹¹ and X¹², at most two of R′ are R′″¹, the rest of R^{m} is R^{c}(preferably, the rest of R^{m} are H).

In another preferred embodiment, M¹ is null, M³ is null or R³, and M² is a ring of formula A; and, in formula A, X⁹ is N or C(R^{m}), X¹⁰ is selected from the group consisting of: O, S, N and C(R^{m}); X¹¹ and X¹² are each independently —C(R^{m})₂—; subscripts m1 and m2 are each independently 1 or 2 (preferably, both of subscripts m1 and m2 are 2).

In another preferred embodiment, M¹ is null, M³ is R³, and M² is a ring of formula A; and, in formula A, X⁹ is N or C(R^{m}), X¹⁰ is N; X¹¹ and X¹² are each independently C(R^{m})₂—; subscripts m1 and m2 are each independently 1 or 2 (preferably, both subscripts m1 and m2 are 2).

In another preferred embodiment, M¹ is null, M³ is R³, and R³ is H, and M² is a ring of formula A; and, in formula A, X⁹ is N, X¹⁰ is N; X¹¹ and X¹² are each independently —C(R^{m})₂—; subscripts m1 and m2 are each independently 1 or 2 (preferably, both subscripts m1 and m2 are 2).

In another preferred embodiment,

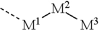

is

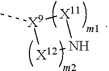

In another preferred embodiment,

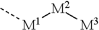

is

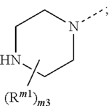

wherein, m3 is 0, 1 or 2.

In another preferred embodiment,

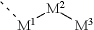

is selected from the group consisting of:

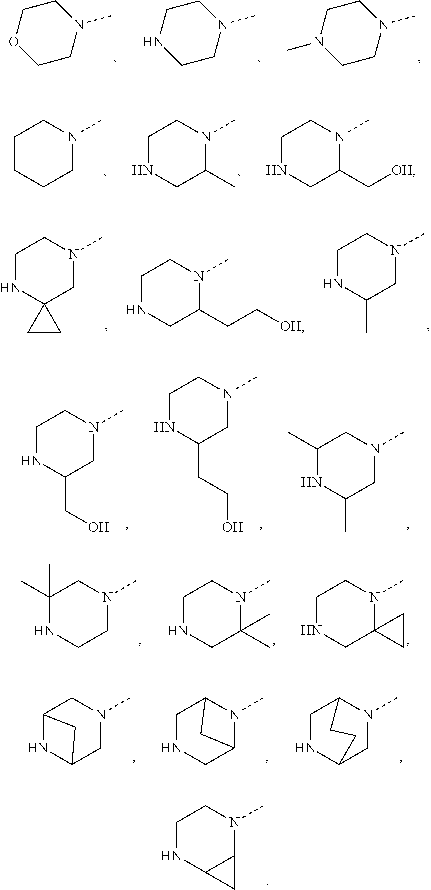

In another preferred embodiment, the compound is of formula I-1

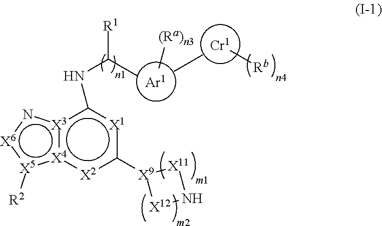

wherein, each group is as defined above.

In another preferred embodiment, M¹ is X⁸ or (M⁴)ₛ, M³ is null or R³, and M² is a ring of formula A; wherein, M⁴ is each independently selected from the group consisting of: O and C₁₋₄ alkylene; s is 1, 2 or 3; and at most one of M⁴ is O.

In another preferred embodiment, M¹ is X⁸, M³ is null or R³, and M² is a ring of formula A.

In another preferred embodiment, X⁸ is N(R^{c}).

In another preferred embodiment, R³ is H.

In another preferred embodiment, m1 and m2 are each independently 1, 2 or 3. In another preferred embodiment, m1 and m2 are each independently 1, 2 or 3 and m1+m2≤4.

In another preferred embodiment, X¹⁰ is N or C(R^{c}), M³ is R³, and R³ is H or C₁₋₆alkyl. In another preferred embodiment, X¹⁰ is N or CH, M³ is R³, and R³ is H.

In another preferred embodiment, X¹⁰ is N, M³ is R³, and R³ is H or C₁₋₆alkyl. In another preferred embodiment, X¹⁰ is N, M³ is R³, and R³ is H.

In another preferred embodiment, in X¹¹ and X¹², only one of R^{m} is R^{m1}, the rest of R^{m} is R^{c}(preferably, the rest of R^{m} are H).

In another preferred embodiment, M¹ is X⁸, M³ is R³, and M² is a ring of formula A; and, in formula A, X⁹ is N or C(R^{m}), X¹⁰ is N or C(R^{m}); X¹¹ and X¹² are each independently selected from the group consisting of: —C(R^{m})₂—, and —N(R^{m})—; subscripts m1 and m2 are each independently 1, 2 or 3 (preferably, m1 and m2 are each independently 1, 2 or 3 and m1+m2≤4).

In another preferred embodiment, M¹ is X⁸, M³ is R³, and M² is a ring of formula A; and, in formula A, X⁹ is N or C(R^{m}), X¹⁰ is N; X¹¹ and X¹² are each independently —C(R^{m})₂—; subscripts m1 and m2 are each independently 1, 2 or 3 (preferably, m1 and m2 are each independently 1, 2 or 3 and m1+m2≤4).

In another preferred embodiment, M¹ is X⁸, X⁸ is N(R^{c}); M³ is R³, and R³ is H, and M² is a ring of formula A; in formula A, X⁹ is C(R^{m}), X¹⁰ is N; X¹¹ and X¹² are each independently —C(R^{m})₂—; subscripts m1 and m2 are each independently 1, 2 or 3 (preferably, m1 and m2 are each independently 1, 2 or 3 and m1+m2≤4).

In another preferred embodiment,

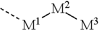

is

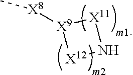

In another preferred embodiment

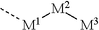

is

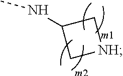

wherein, subscripts m1 and m2 are each independently 1, 2 or 3 (preferably, m1 and m2 are each independently 1, 2 or 3 and m1+m2≤4).

In another preferred embodiment,

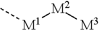

is selected from the group consisting of:

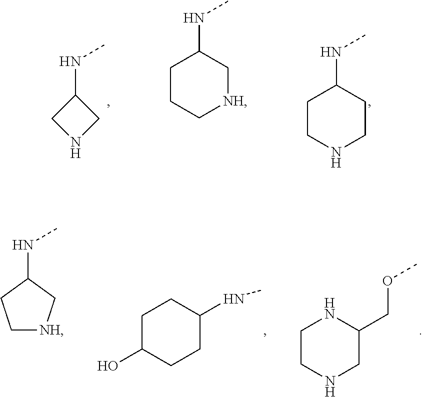

In another preferred embodiment, the compound is of formula I-2

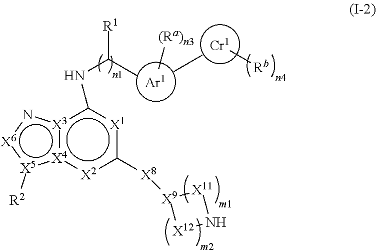

wherein, each group is as defined above.

In another preferred embodiment, M¹ is null, M³ is —NH—R³, and M² is a ring of formula A.

In another preferred embodiment, m1 and m2 are each independently 1, 2 or 3. In another preferred embodiment, m1 and m2 are each independently 1, 2 or 3 and m1+m2≤4.

In another preferred embodiment, R³ is H.

In another preferred embodiment, X⁹ is N.

In another preferred embodiment, X¹⁰ is C(R^{m}). In another preferred embodiment, X¹⁰ is C(R^{c}). In another preferred embodiment, X¹⁰ is CH.

In another preferred embodiment, X¹¹ and X¹² are each independently —C(R^{m})₂—. In another preferred embodiment, X¹¹ and X¹² are each independently —C(R^{c})₂—. In another preferred embodiment, X¹¹ and X¹² are each independently —CH₂—.

In another preferred embodiment, M¹ is null, M³ is —NH—R³, and M² is a ring of formula A; and, in formula A, X⁹ is N or C(R^{m}), X¹⁰ is C(R^{m}); X¹¹ and X¹² are each independently —C(R^{m})₂—; subscripts m1 and m2 are each independently 1, 2 or 3 (preferably, m1 and m2 are each independently 1, 2 or 3 and m1+m2≤4).

In another preferred embodiment, M¹ is null, M³ is —NH—R³, and M² is a ring of formula A; and, in formula A, X⁹ is N, X¹⁰ is C(R^{m}); X¹¹ and X¹² are each independently —C(R^{m})₂—; subscripts m1 and m2 are each independently 1, 2 or 3 (preferably, m1 and m2 are each independently 1, 2 or 3 and m1+m2≤4).

In another preferred embodiment, M¹ is null, M³ is —NH—R³, and M² is a ring of formula A; and, in formula A, X⁹ is N, X¹⁰ is C(R^{c}); X¹¹ and X¹² are each independently is —C(R^{c})₂—; subscripts m1 and m2 are each independently 1, 2 or 3 (preferably, m1 and m2 are each independently 1, 2 or 3 and m1+m2≤4).

In another preferred embodiment,

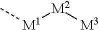

is

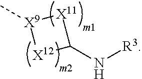

In another preferred embodiment,

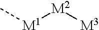

is

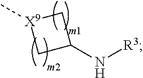

wherein, subscripts m1 and m2 are each independently 1, 2 or 3 (preferably, m1 and m2 are each independently 1, 2 or 3 and m1+m2≤4).

In another preferred embodiment,

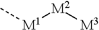

is selected from the group consisting of:

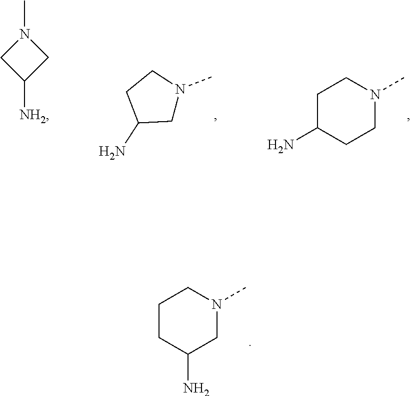

In another preferred embodiment, the compound is of formula I-3

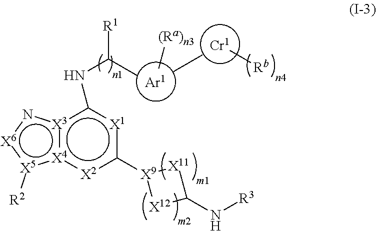

wherein, each group is as defined above.

In another preferred embodiment, M¹ is X⁸, M³ is —NH—R³, and M² is a ring of formula A.

In another preferred embodiment, X⁸ is N(R^{c}).

In another preferred embodiment, R³ is H.

In another preferred embodiment, m1 and m2 are each independently 1, 2 or 3. In another preferred embodiment, m1 and m2 are each independently 1, 2 or 3 and m1+m2≤4.

In another preferred embodiment, X⁹ is C(R^{m}); preferably, X⁹ is C(R^{c}); preferably, X⁹ is CH.

In another preferred embodiment, X¹⁰ is C(R^{m}); preferably, X¹⁰ is C(R^{c}); preferably, X¹⁰ is CH.

In another preferred embodiment, X¹¹ and X¹² are each independently —C(R^{m})₂—. In another preferred embodiment, X¹¹ and X¹² are each independently —C(R^{c})₂—. In another preferred embodiment, X¹¹ and X¹² are each independently —CH₂—.

In another preferred embodiment, M¹ is X⁸, M³ is —NH—R³, and M² is a ring of formula A; and, X⁸ is N(R^{c}) or C(R^{c})₂; in formula A, X⁹ is C(R^{c}), X¹⁰ is C(R^{c}); X¹¹ and X¹² are each independently —C(R^{c})₂—; subscripts m1 and m2 are each independently 1, 2 or 3 (preferably, m1 and m2 are each independently 1, 2 or 3 and m1+m2≤4). In another preferred embodiment, M¹ is X⁸, M³ is —NH—R³, and M² is a ring of formula A; and, X⁸ is N(R^{c}); in formula A, X⁹ is C(R^{c}), X¹⁰ is C(R^{c}); X¹¹ and X¹² are each independently —C(R^{c})₂—; subscripts m1 and m2 are each independently 1, 2 or 3 (preferably, m1 and m2 are each independently 1, 2 or 3 and m1+m2≤4).

In another preferred embodiment, M¹ is X⁸, M³ is —NH—R³, and M² is a ring of formula A; and, X⁸ is NH; in formula A, X⁹ is CH, X¹⁰ is CH; X¹¹ and X¹² are each independently —CH₂—; subscripts m1 and m2 are each independently 1, 2 or 3 (preferably, m1 and m2 are each independently 1, 2 or 3 and m1+m2≤4).

In another preferred embodiment

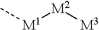

is

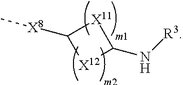

In another preferred embodiment,

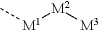

is

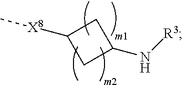

wherein, subscripts m1 and m2 are each independently 1, 2 or 3 (preferably, m1 and m2 are each independently 1, 2 or 3 and m1+m2≤4).

In another preferred embodiment,

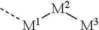

is selected from the group consisting of:

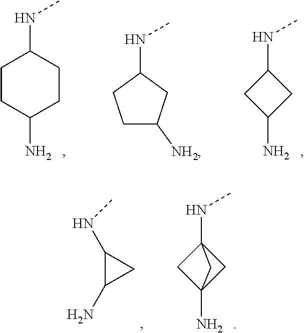

In another preferred embodiment, the compound is of formula I-4

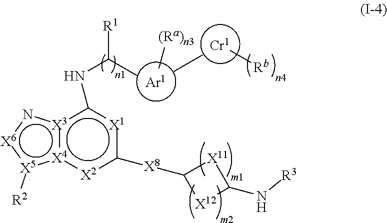

wherein, each group is as defined above.

In another preferred embodiment, the compound is a compound of formula I-1, wherein,

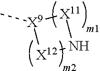

is

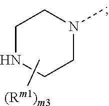

m3 is 0, 1 or 2.

In another preferred embodiment, R¹, R², R³, X¹, X², X³, X⁴, X⁵, X⁶, X⁷, X⁸, X⁹, X¹⁰, X¹¹, X¹², M¹, M², M³, M⁴, subscript n1, subscript n3, subscript n4, subscript n5, subscript n6, subscript m1, subscript m2, subscript m3, subscript s, Ar¹, Ar², Cr¹, R^{a}, R^{b}, R^{c}, R^{e}, R^{f}, R^{m}, R^{m1}, R, R′, R″ and R′″ are each independently the corresponding group in example compounds or specific compounds shown in Table A1, A2, A3 and A4.

In another preferred embodiment, the compound is a compound selected from Table A1, A2, A3 and Table A4.

In the Second aspect of the present invention, provided is a pharmaceutical composition comprising
(i) the compound according to the first aspect, or the pharmaceutically acceptable salt thereof; and
(ii) pharmaceutically acceptable carriers.

In the third aspect of the present invention, provided is a use of the compound of formula (I) according to the first aspect, or the pharmaceutically acceptable salt thereof in the preparation of a medicament for treating cancer.

In the forth aspect of the present invention, provided is a method for treating cancer comprising a step of administering to a subject in need thereof a safe and effective amount of the compound of formula (I) according to the first aspect or the pharmaceutically acceptable salt thereof.

In the fifth aspect of the present invention, provided is a method for degrading Cyclin K comprises a step of: treating a subject with the compound of formula (I) according to the first aspect, thereby degrading Cyclin K.

In another preferred embodiment, the subject is a cell.

In another preferred embodiment, the subject is a HepG2 cell.

In another preferred embodiment, the method is in vitro and non-therapeutic.

In the sixth aspect of the present invention, provided is a conjugate or a pharmaceutically acceptable salt thereof, wherein the conjugate is a conjugate formed by the compound of formula (I) according to the first aspect and a polypeptide element or a targeting ligand.

In another preferred embodiment, the conjugate is of formula II,

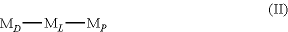

wherein,
M_{D} is a moiety derived from the compound of formula (I) according to the first aspect;
M_{L} is null or a linker moiety for connecting M_{D} and M_{P};
M_{P} is a moiety derived from the polypeptide element or the targeting ligand.

In another preferred embodiment, the targeting ligand refers to a small molecule that is capable of binding to an extracellular receptor.

In another preferred embodiment, the polypeptide element includes (but not limited to): peptide, antibody, antibody fragment, fusion protein, or combinations thereof.

In another preferred embodiment, M_{P} is selected from the group consisting of: peptide, antibody, antibody fragment, fusion protein, or a moiety of small molecule ligand that is capable of binding to extracellular receptor.

In another preferred embodiment, the antibody includes (but is not limited to): nanobody, small molecule antibody (minibody), antibody fragment (such as scFv, Fab), double antibody (Dibody), monoclonal antibody (mAb), or combinations thereof.

In another preferred embodiment, the polypeptide (e.g., targeting polypeptide) includes but is not limited to: EGFR, FGFR, SSTR1-14, GnRH, TRPV1-6, RGD, iRGD, EphA2, or combinations thereof.

In another preferred embodiment, the targets that the small molecule ligand can bind to include (but are not limited to): FR, HSP90, PSMA, ASGPR, or combinations thereof.

In another preferred example, the antibody can bind to an antigen or receptor selected from the group consisting of (for example, bind to one (i.e., monofunctional antibody) or two (i.e., bifunctional antibody) or more (i.e., multifunctional antibody) antigens and/or receptors selected from the group consisting of): DLL3, EDAR, CLL1, BMPR1B, E16, STEAP1, 0772P, MPF, 5T4, NaPi2b, Sema 5b, PSCA hlg, ETBR, MSG783, STEAP2, TrpM4, CRIPTO, CD21, CD22, CD79b, CD19, CD37, CD138, FcRH2, B7-H4, HER2, NCA, MDP, IL20Rα, Brevican, EphB2R, ASLG659, PSCA, GEDA, BAFF-R, CD79a, CXCR5, HLA-DOB, P2X5, CD72, LY64, FcRH1, IRTA2, TENB2, PMEL17, TMEFF1, GDNF-Ra1, Ly6E, TMEM46, Ly6G6D, LGR5, RET, LY6K, GPR19, GPR54, ASPHD1, Tyrosinase, TMEM118, GPR172A, MUC1, CD70, CD71, MUC16, methothelin, FOLR1, Trop-2, gpNMB, EGFR, ENPP3, PSMA, CA6, GPC-3, PTK7, CD44, CD56, TIM-1, Cadherin-6, ASG-15ME, ASG-22ME, CanAg, AXL, CEACAM5, EphA4, cMet, FGFR2, FGFR3, CD123, Her3, LAMP1, LRRC15, TDGF1, CD66, CD25, BCMA, GCC, Noch3, cMet, EGFR and CD33, or receptors such as CD70, Trop2, PD-L1, CD47, CLDN-18.2.

In another preferred embodiment, the targeting ligand can also bind to receptors that can be targeted by specific small molecules, such as folic acid, HSP90, glucose transporter 1 (GLUT1), aminopeptidase N (APN), low-density lipoprotein receptor-related protein 1 (LRP1), prostate-specific membrane antigen (PSMA), integrin αvβ3, bombesin receptor, somatostatin receptor (SSTR), tumor hypoxic microenvironment, and carbonic anhydrase IX (CAIX) and other receptors.

In the seventh aspect of the invention, provided is a pharmaceutical composition comprising
(i) the conjugate according to the sixth aspect, or the pharmaceutically acceptable salt thereof; and
(ii) pharmaceutically acceptable carriers.

In the eighth aspect of the present invention, provided is a use of the conjugate according to the sixth aspect, or the pharmaceutically acceptable salt thereof in the preparation of a medicament for treating cancer.

In the ninth aspect of the present invention, provided is a method for treating cancer comprising a step of: administering to a subject in need thereof a safe and effective amount of the conjugate according to the sixth aspect, or the pharmaceutically acceptable salt thereof.

It should be understood that within the scope of the present invention, the above technical features of the present invention and the technical features specifically described in the following (e.g., embodiments) can be combined with each other, thereby forming a new or preferred technical solution. Due to space limitations, it will not be repeated herein.

### DESCRIPTION OF THE DRAWINGS

FIG. 1 shows the immunohistochemical staining (IHC) results of compounds CR-8, UB-018, UB-022, and UB-027.

FIG. 2 shows the degradation effects of CR-8 and the compounds of the present invention on Cyclin K in HEK293 cells.

### EMBODIMENTS FOR CARRYING OUT THE INVENTION

After extensive and in-depth research, the inventors unexpectedly discovered a class of compounds with novel structures (such as the compounds of formula (I), formula (I-1), formula (I-2), formula (I-3) or formula (I-4) in this article, especially when there is no N atom on the Cr¹ ring), which also have excellent effects in inducing the degradation of Cyclin K by modifying functional groups at other positions of the core structure. Based on this, the inventors completed the present invention.

### Terms

Unless otherwise indicated, the bonds represented by dashed lines in each structural formula represent the position of attachment to other parts.

As used herein, the term “alkyl”, by itself or as part of another substituent, means, unless otherwise stated, a straight or branched chain hydrocarbon radical, having the number of carbon atoms designated (i.e. C₁₋₆ means 1-6 carbons). Preferably, alkyl contains 1 to 4 carbons i.e. C₁₋₄alkyl. Examples of alkyl groups include, but are not limited to, methyl, ethyl, n-propyl, isopropyl, n-butyl, t-butyl, iso-butyl, sec-butyl, n-pentyl, n-hexyl, n-heptyl, n-octyl, and the like. The term “alkenyl” refers to an unsaturated alkyl group having one or more double bonds. Preferably, alkenyl contains 2 to 4 carbons i.e., C₂₋₄alkenyl. Similarly, the term “alkynyl” refers to an unsaturated alkyl group having one or more triple bonds. Preferably, alkynyl contains 2 to 4 carbons i.e., C₂₋₄alkynyl. Examples of such unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

Unless otherwise stated, the term “heteroalkyl”, by itself or in combination with other terms, refers to a stable straight or branched group formed by an alkyl group as defined above in which one or more (e.g., 1 or 2) carbon atoms are replaced by, for example, a heteroatom selected from O, N, Si and S, and wherein the nitrogen and sulfur atoms are optionally oxidized and the nitrogen heteroatom is optionally quaternized. The heteroatom(s) O, N and S may be placed at any interior position of the heteroalkyl group. The heteroatom Si may be placed at any position of the heteroalkyl group, including the position at which the alkyl group is attached to the remainder of the molecule.

The term “alkylene”, by itself or as part of another substituent, means a divalent group derived from an alkane, for example, —CH₂. Preferably, in the present application, with 1, 2, 3 or 4 carbon atoms (i.e., C₁₋₄ alkylene).

The term “heteroalkylene”, by itself or as part of another substituent, means a divalent group derived from a hereroalkane.

As used herein, the term “carbocyclyl” or “carbocycle” refers to a hydrocarbon ring (hydrocarbyl) having an indicated number of ring atoms (e.g., C₃₋₁₀ carbocycle(carbocyclyl), C₄₋₁₀ carbocycle(carbocyclyl), C₄₋₆ carbocycle(carbocyclyl)) and being fully saturated or having one or two double bond between ring vertices. This term is also meant to contain bicyclic and polycyclic hydrocarbon rings such as, for example, bicyclo[2.2.1]heptane, bicyclo[2.2.2]octane, etc. The term “heterocyclyl” or “heterocycle” refers to a carbocycle (carbocyclyl) that contain from one to five heteroatoms selected from N, O, and S, wherein the nitrogen and sulfur atoms are optionally oxidized, and the nitrogen atom(s) are optionally quaternized. The heterocycle (heterocyclyl) can be a monocyclic, bicyclic or polycyclic ring system, preferably a monocyclic ring. Non limiting examples of heterocycle (heterocyclyl) include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, and the like. The heterocycle (heterocyclyl) can be attached to the rest of the molecule via a ring carbon or a heteroatom.

The term “cycloalkyl” refers to a hydrocarbon ring with a specified number of ring atoms (e.g., C₃₋₆ cycloalkyl) and is completely saturated. Cycloalkyl groups can be monovalent or divalent.

The terms “alkoxy” is used in their conventional meaning, and refers to those alkyl groups attached to the rest of the molecule via an oxygen atom, amino, or a sulfur atom, respectively.

The terms “halo” or “halogen,” by themselves or as part of another substituent, mean, unless otherwise stated, fluorine, chlorine, bromine, or iodine atom. Additionally, terms such as “haloalkyl,” are meant to include monohaloalkyl or polyhaloalkyl. For example, the term “C₁₋₄ haloalkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon group which can be a monocycle or polycyclic rings (up to three rings) which are fused together or linked covalently. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom.

Non-limiting examples of aryl include phenyl, naphthyl, and biphenyl, while non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiazolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. Substituents for above-stated aryl and heteroaryl ring systems are selected from the group of acceptable substituents described below.

The above terms (e.g., “alkyl,” “aryl” and “heteroaryl”), in some embodiments, will include both substituted and unsubstituted forms of the indicated radical. The preferred substituents for each type of group are provided below. For brevity, the terms aryl and heteroaryl will refer to substituted or unsubstituted versions as provided below, while the term “alkyl” and related aliphatic radicals are meant to refer to unsubstituted version, unless indicated to be substituted.

substituents for the alkyl radicals (including those groups often referred to as alkylene, alkenyl, alkynyl and cycloalkyl) can be a variety of groups selected from: -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, NR′S(O)₂R″, —CN and —NO₂, in a number ranging from zero to (2M′+1), where M′ is the total number of carbon atoms in such radical. R′, R″ and R′″ each independently refer to hydrogen, unsubstituted C₁₋₈ alkyl, unsubstituted heteroalkyl, unsubstituted aryl, aryl substituted with 1-3 halogens, unsubstituted C₁₋₈ alkyl, C₁₋₈ alkoxy or C₁₋₈ thioalkoxy groups, or unsubstituted aryl-C₁₋₄ alkyl groups. When R′ and R″ are attached to the same nitrogen atom, they can combine with the nitrogen atom to form a 3-, 4-, 5-, 6-, or 7-membered ring. For example, —NR′R″ is meant to include 1-pyrrolidinyl and 4-morpholinyl. The term “acyl” as used by itself or as part of another group refers to groups wherein two substitutents on the carbon that is closest to the point of attachment for the radical is replaced with the substitutent ═O (e.g., C(O)CH₃, —C(O)CH₂CH₂OR′ and the like).

Similarly, substituents for the aryl and heteroaryl groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —NR'S(O)₂R″, —N₃, perfluoro(C₁-C₄)alkoxy and perfluoro(C₁-C₄)alkyl, in a number ranging from 0 to the total number of open valences on the aromatic ring system; and wherein R′, R″ and R′″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C-4 alkyl. Other suitable substituents include each of the above aryl substituents attached to a ring atom by an alkylene tether of from 1-4 carbon atoms.

As used herein, the term “heteroatom” is meant to include oxygen (O), nitrogen (N), sulfur (S) and silicon (Si).

For the compounds provided herein, a bond that is drawn from a substituent (typically an R group) to the center of an aromatic ring (e.g., benzene, pyridine, and the like) will be understood to refer to a bond providing a connection at any of the available vertices of the aromatic ring. In some embodiments, the depiction will also include connection at a ring which is fused to the aromatic ring. For example, a bond drawn to the center of the benzene portion of an indole, will indicate a bond to any available vertex of the six- or five-membered ring portions of the indole.

As used herein, “moiety derived from . . . ” refers to a moiety or fragment of an active substance remaining after an active substance (e.g., a polypeptide element such as an antibody or a targeting ligand) is subject to certain means (e.g., reacting an active group thereon, or introducing an active group thereon for reaction) to form a linking group with other moiety, and the moiety or fragment retains the function of the active substance (e.g., the ability to target a desired receptor). Specifically, the linking groups formed by “derived” include but are not limited to: —NH—, —CONH—, —CO—, —S—S—, and the like.

The term “pharmaceutically acceptable salts” is meant to include salts prepared from the active compounds and relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either solvent-free or in a suitable inert solvent. Examples of salts derived from pharmaceutically acceptable inorganic bases include aluminum, ammonium, calcium, copper, iron, ferrous, lithium, magnesium, manganese, manganous, potassium, sodium, zinc, and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, halamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either solvent-free or in a suitable inert solvent. Examples of pharmaceutically acceptable acid addition salts include those derived from inorganic acids like hydrochloric acid, hydrobromic acid, nitric acid, carbonic acid, monohydrogencarbonic acid, phosphoric acid, monohydrogenphosphoric acid, dihydrogenphosphoric acid, sulfuric acid, monohydrogensulfuric acid, hydriodic acid, or phosphorous acid and the like, as well as the salts derived from relatively nontoxic organic acids like acetic acid, propionic acid, isobutyric acid, malonic acid, benzoic acid, succinic acid, suberic acid, fumaric acid, mandelic acid, phthalic acid, benzenesulfonic acid, p-tolylsulfonic acid, citric acid, tartaric acid, methanesulfonic acid, and the like. Also included are salts of amino acids such as arginate and the like, and salts of organic acids like glucuronic acid or galactunoric acid and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms thereof in certain physical properties, such as solubility in polar solvents, but in addition to the above, those salts are equivalent to the parent form of the compound for the purposes of the present invention.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, when placed in a transdermal patch reservoir containing suitable enzymes or chemical reagents, the prodrug can be slowly converted to the compound of the invention.

Certain compounds of the present disclosure can exist in unsolvated forms as well as solvated forms, including hydrated forms. The solvated forms are generally equivalent to the non-solvated forms and should be included in the scope of the present invention. Certain compounds of the present disclosure may exist in polymorph or amorphous forms. Generally, as for the application considered in the present invention, all physical forms are equivalent and should be included in the scope of the present invention.

Certain compounds of the present disclosure possess asymmetric carbon atoms (optical centers) or double bonds; the racemates, diastereomers, geometric isomers, regioisomers and individual isomers (e.g., separate enantiomers) are all intended to be encompassed within the scope of the present disclosure. When compounds are provided herein with an identified stereochemistry (indicated as R or S, or with dashed or wedge bond designations), those compounds will be understood by one of skill in the art to be substantially free of other isomers (e.g., at least 80%, 90%, 95%, 98%, 99%, and up to 100% free of the other isomer).

The compounds of the present disclosure may also contain unnatural proportions of atomic isotopes at one or more of isotopic atoms that constitute such compounds. The unnatural proportions of certain isotope can be defined as the amount from the naturally found amount of the atom discussed to 100% of that atom. For example, the compounds may incorporate radioactive isotopes, such as for example tritium (3H), iodine-125 (¹²⁵I) or carbon-14 (¹⁴C), or non-radioactive isotopes, such as deuterium (2H) or carbon-13 (¹³C). Such isotopic variants may provide additional uses in addition to those described in this application.

For instance, isotopic variants of the compounds of the disclosure may find additional utility, including but not limited to, as diagnostic and/or imaging reagents, or as cytotoxic/radiotoxic therapeutic agents. Additionally, isotopic variants of the compounds of the disclosure can have altered pharmacokinetic and pharmacodynamic characteristics which can contribute to enhanced safety, tolerability or efficacy during treatment. All isotopic variations of the compounds of the present disclosure, whether radioactive or not, should be encompassed within the scope of the present disclosure.

### Molecular Glue Degrader

Current studies have found that the Cyclin-dependent kinase (CDK) inhibitor CR8 is also a molecular glue degrader. CR8 induces CDK12/cyclin K to directly form a complex with CUL4/DDB1, causing cyclin K to be ubiquitinated and degraded through the proteasome system, thereby killing cancer cells more effectively.

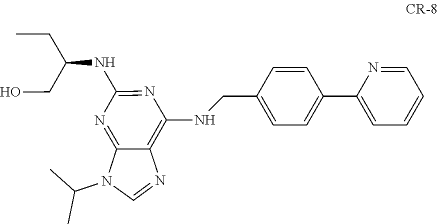 [SMILES: CC[C@H](CO)Nc1nc(NCc2ccc(-c3ccccn3)cc2)c2ncn(C(C)C)c2n1]

Further analysis of the structure of the protein-small molecule-protein complex CUL4-RBX1-DDB1-CR8-CDK12-cyclin revealed that CDK12 plays a role similar to the CRBN substrate receptor. The presence and correct orientation of CDK12 surface and the 2-pyridine moiety of the CR8 increase the gain of function of CR8, leading to the degradation of cyclin K. That is, CR8-phenylpyridine endows it with the activity of a molecular glue, induces cyclin K degradation, and increases the toxicity of CR8.

By modifying the structure of CR8, the authors found that the activity of the CR8 molecular glue mainly depends on the 2-pyridine moiety exposed on the kinase surface. They concluded that this chemical group enables CR8 to function like a molecular glue degrader. Therefore, chemical modification of the exposed portion of the inhibitor on the protein surface can turn them into molecular glue degraders targeting specific protein targets.

However, the inventors found in their research that the compounds having the structures shown in the present invention, formula (I), formula (I-1), formula (I-2), formula (I-3), and formula (I-4), especially the structure shown in formula (I-1), still have excellent effects in inducing degradation of cyclins such as cyclin K in the presence or absence of pyridine substituents (or other nitrogen-containing heteroaromatic ring substituents) or whether they are pyridine substituents (or other nitrogen-containing heteroaromatic ring substituents), and based on this, the inventors provide a series of novel cyclin regulators (more specifically molecular glue degraders).

In one embodiment, provided is a compound of formula (I), or a pharmaceutically acceptable salt thereof,

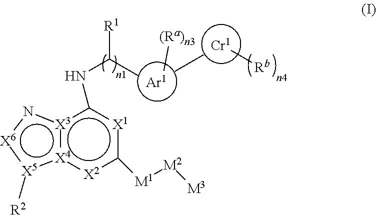

wherein, each group is as defined in the first aspect.

In another preferred embodiment, provided is a compound of formula (I-1), (I-2), (I-3) or (I-4), or a pharmaceutically acceptable salt thereof;

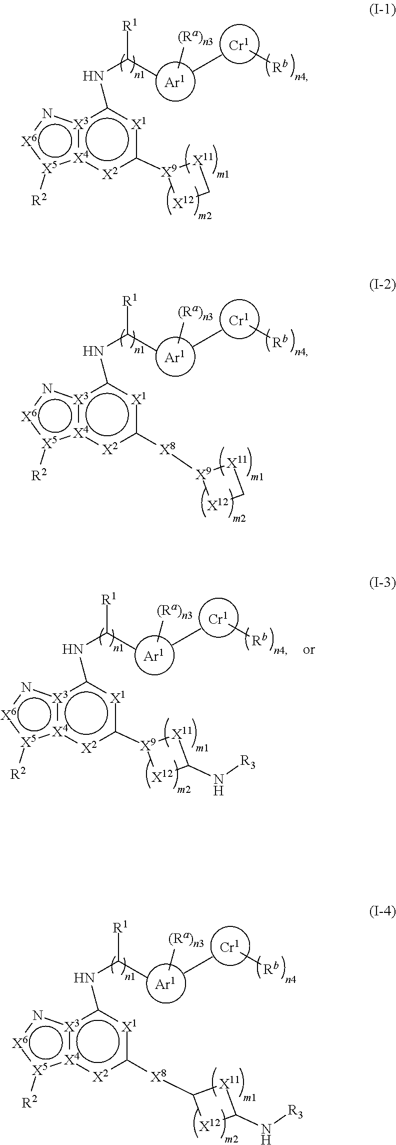

wherein, each group is defined as above.

In one aspect of the present invention, provided is a molecular glue degrader as shown in any of the following

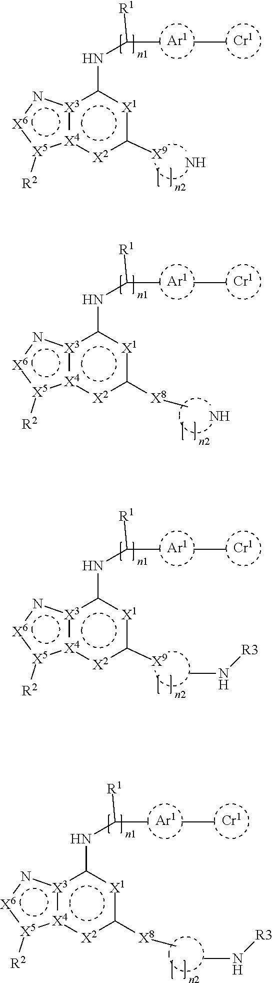

wherein, n2 is 2- to 4 (i.e., the ring in which n2 is located is a 4- to 6-membered ring), Ring Ar¹ may further be optionally substituted with n3 R^{a} substituents (not shown), and Ring Cr¹ may further be optionally substituted with n4 R^{b} substituents (not shown); R¹, R², R³, X¹, X², X³, X⁴, X⁵, X⁶, X⁸, X⁹, subscripts n1, n3, n4, Ar¹, Cr¹, R^{a} and R^{b} are as defined elsewhere herein.

In another aspect of the present invention, provided is a molecular glue degrader as shown in any of the following

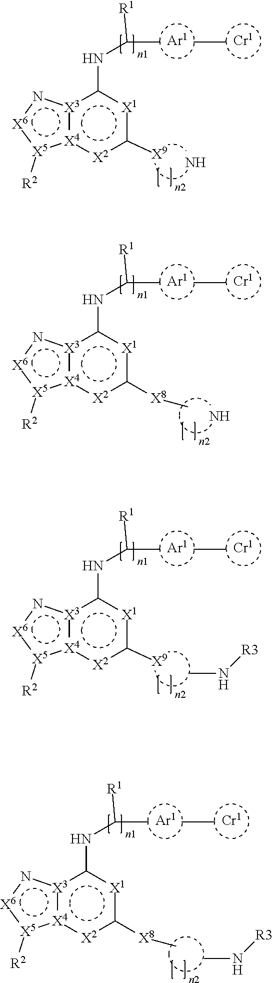

wherein n2 is 2- to 4 (i.e., the ring in which n2 is located is a 4- to 6-membered ring), Ring Ar¹ may further be optionally substituted with n3 R^{a} substituents (not shown), and Ring Ar² may further be optionally substituted with n4 R^{b} substituents (not shown); R¹, R², R³, X¹, X², X³, X⁴, X⁵, X⁶, X⁸, X⁹, subscripts n1, n3, n4, Ar¹, Cr¹, R^{a} and R^{b} are as defined elsewhere herein.

### Active Ingredients

As used herein, the term “compound of the invention” refers to the compound of formula (I). The term also comprises the crystal forms, or pharmaceutically acceptable salts of compound of formula (I).

As used herein, an active ingredient may also be a conjugate formed by the compound of formula (I) with an antibody or a polypeptide.

### Pharmaceutical Compositions and Administration Method

Because the compound of the present invention has excellent activity in inducing degradation of Cyclin K, the compound of the present invention and its various crystal forms, pharmaceutically acceptable inorganic or organic salts, hydrates or solvates, and pharmaceutical compositions containing the compound of the present invention as the main active ingredient, or the conjugate formed by the compound of the invention with an antibody or a polypeptide can be used to treat, prevent Cyclin K-related diseases or Cyclin K-involved diseases. According to the prior art, the compounds of the invention can be used to treat the following diseases: cancer, etc.

The pharmaceutical composition of the invention comprises the compound of the present invention or the pharmaceutically acceptable salts thereof in a safe and effective dosage range, and pharmaceutically acceptable excipients or carriers. In which, the term “safe and effective amount” means that the amount of the compound is sufficient to significantly improve the condition without causing serious side effects.

“Pharmaceutically acceptable carrier” means one or more compatible solid or liquid fillers, or gelatinous materials which are suitable for human use and should be of sufficient purity and sufficiently low toxicity. “Compatibility” used herein means that the components of the composition can be admixed with the compounds of the invention and with each other without significantly reducing the efficacy of the compounds. Some examples of pharmaceutically acceptable carriers include cellulose and the derivatives thereof (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agent (such as sodium dodecyl sulfate), coloring agents, flavoring agents, stabilizers, antioxidants, preservatives, pyrogen-free water, etc.

There is no special limitation on the administration mode for the compound or pharmaceutical compositions of the present invention, and the representative administration mode includes (but is not limited to): oral, intratumoral, rectal, parenteral (intravenous, intramuscular or subcutaneous), and topical administration.

Solid dosage forms for oral administration include capsules, tablets, pills, powders and granules. In these solid dosage forms, the active compounds are mixed with at least one conventional inert excipient (or carrier), such as sodium citrate or CaHPO₄, or mixed with any of the following components: (a) fillers or compatibilizer, for example, starch, lactose, sucrose, glucose, mannitol and silicic acid; (b) binders, for example, hydroxymethyl cellulose, alginates, gelatin, polyvinylpyrrolidone, sucrose and arabic gum; (c) humectant, such as, glycerol; (d) disintegrating agents such as agar, calcium carbonate, potato starch or tapioca starch, alginic acid, certain composite silicates, and sodium carbonate; (e) dissolution-retarding agents, such as paraffin; (f) absorption accelerators, for example, quaternary ammonium compounds; (g) wetting agents, such as cetyl alcohol and glyceryl monostearate; (h) adsorbents, for example, kaolin; and (i) lubricants such as talc, stearin calcium, magnesium stearate, solid polyethylene glycol, sodium lauryl sulfate, or the mixtures thereof. In capsules, tablets, and pills, the dosage form may also include a buffer.

Solid dosage forms such as tablets, sugar pills, capsules, pills, and granules can be prepared using coating and shell materials, such as casings and other well-known materials in the art. They can contain an opaque agent. The release of the active compounds or compounds in the compositions can be released in a delayed mode in a given portion of the digestive tract. Examples of embedding components that can be used are polymeric substances and waxes. If necessary, the active compound can also form microcapsules with one or more of the aforementioned excipients.

Liquid dosage forms for oral administration include pharmaceutically acceptable emulsions, solutions, suspensions, syrups or tinctures. In addition to the active compound, the liquid dosage form may contain inert diluents conventionally used in the art, such as water or other solvents, solubilizers and emulsifiers, such as ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil or mixtures of these substances.

In addition to these inert diluents, the composition can also include additives such as wetting agents, emulsifiers and suspending agents, sweeteners, corrigents, and spices.

In addition to active compounds, suspensions can include suspending agents such as ethoxylated isooctadecanol, polyoxyethylene sorbitol and dehydrated sorbitol esters, microcrystalline cellulose, methanol aluminum and agar, or mixtures of these substances.

The compositions for parenteral injection may comprise physiologically acceptable sterile aqueous or anhydrous solutions, dispersions, suspensions or emulsions, and sterile powders which can be re-dissolved into sterile injectable solutions or dispersions. Suitable aqueous and non-aqueous carriers, diluents, solvents or excipients include water, ethanol, polyols, and their suitable mixtures.

The dosage forms of the compounds of the present invention used for local administration include ointments, powders, patches, sprays, and inhalants. The active ingredients are mixed under sterile conditions with physiologically acceptable carriers and any preservatives, buffers, or propellants that may be necessary.

The compound of the present invention can be administered separately or in combination with other pharmaceutically acceptable compounds.

When the pharmaceutical composition is used, a safe and effective amount of the compound of the present invention is administered to a mammal (such as a human) in need of treatment, wherein the dosage at the time of administration is the pharmaceutically effective dosage, for people having a body weight of 60 kg, the daily dose is usually 1-2000 mg, preferably 20-500 mg. Of course, the specific dosage should also consider factors such as the route of administration and the patient's health status, which are within the skill range of a skilled physician.

### Polypeptide Element

As used herein, the term “polypeptide element” includes peptide fragments (such as oligopeptide comprising 3-20 aa) or proteins. In addition, this term also includes intact proteins or fragments thereof. Preferred polypeptide elements include antibodies (such as intact antibodies, single-chain antibodies, nanobodies, antibody fragments), especially those antibodies against tumor cell markers (such as tumor markers located on the surface of tumor cells, such as receptors on the cell surface) or inflammatory factors (such as inflammatory factors associated with autoimmune diseases).

As used herein, term “antibody” or “immunoglobulin” is a heterotetrameric glycoprotein of about 150,000 daltons with the same structural characteristics, which consists of two identical light chains (L) and two identical heavy chains (H). Each light chain is connected to the heavy chain by a covalent disulfide bond, and the number of disulfide bonds between the heavy chains of different immunoglobulin isotypes are different. Each heavy and light chain also has regularly spaced intrachain disulfide bonds. Each heavy chain has a variable region (VH) at one end, followed by multiple constant regions. There are a variable region (VL) at one end of each light chain and a constant region at the other end. The constant region of the light chain is relative to the first constant region of the heavy chain, and the variable region of the light chain is relative to the variable region of the heavy chain. Special amino acid residues form an interface between the variable regions of the light chain and the heavy chain.

As used herein, terms “single-domain antibody” and “nanobody” have the same meaning, and refer to cloning the variable region of the heavy chain of an antibody, and constructing a single-domain antibody consisting of only one heavy chain variable region, which is the smallest antigen-binding fragment that having complete functions. Usually, after obtaining an antibody naturally missing constant region 1 (CH1) of light chain and heavy chain, the variable region of the heavy chain of the antibody is cloned to construct a single domain antibody consisting of only one heavy chain variable region.

As used herein, term “variable” means that certain parts of the variable region of the antibody are different in sequence, which forms the binding and specificity to specific antigens of various specific antibodies. However, variabilities are not evenly distributed throughout the variable regions of antibodies. It is concentrated in three fragments that are called complementarity determining regions (CDR) or hypervariable regions in the variable regions of light chain and heavy chain. More conservative parts of the variable region are called the framework region (FR). The variable regions of the natural heavy and light chains each contain four FR regions, which are in a roughly β-folded conformation and are linked by three CDRs that form a linking loop, which in some cases can form a partially folded structure. The CDRs in each chain are closely placed together through the FR regions and form the antigen binding site of the antibody together with the CDRs in the other chain. Constant regions do not directly participate in the binding of antibodies to antigens, but they exhibit different effector functions, such as participating in antibody-dependent cytotoxicity of antibodies.

The “light chains” of vertebrate antibodies (immunoglobulins) can be classified in one of two distinct categories (called K and k) based on the amino acid sequence of constant regions thereof. According to the amino acid sequence of the constant region in heavy chain thereof, immunoglobulins can be classified into different types. There are five main classes of immunoglobulins: IgA, IgD, IgE, IgG and IgM, some of which can be further classified into subclasses (isotypes), such as IgG1, IgG2, IgG3, IgG4, IgA and IgA2. The constant regions in heavy chains corresponding to different classes of immunoglobulins are called α, δ, ε, γ, and μ respectively. The subunit structures and three-dimensional configurations of different classes of immunoglobulins are well known to those skilled in the art.

Generally, the antigen-binding properties of antibodies can be described by 3 specific regions located in the variable regions of the heavy and light chains, called variable regions (CDR), which divide the region into 4 framework regions (FRs). The amino acid sequence of 4 FRs is relatively conservative and does not directly participate in the binding reaction. These CDRs form a circular structure, and the β-pleated sheet formed by the FRs in between are close to each other in space structure, and the CDRs on the heavy chain and the corresponding CDRs on the light chain constitute the antigen binding site of the antibody. It can be determined by comparing the amino acid sequences of antibodies of the same type which amino acids constitute the FR or CDR regions.

In the present invention, the polypeptide elements can include not only intact antibodies, but also fragments of antibodies with immunological activity (such as Fab or (Fab′)₂ fragment; heavy chain of antibodies; or light chain of antibodies) or fusion proteins formed by antibodies and other sequences. Therefore, the present invention also includes fragments, derivatives and analogs of the antibodies.

### Targeting Ligand

Targeting ligands (or moiety of target protein or target protein ligand or ligand) are small molecules that capable of binding to interesting target protein.

In some embodiments of the present application, the targeting ligand may be (or derived from) a target molecule.

Some embodiments of this application relate to target molecules. Representative target molecules include but are not limited to: folic acid, Hsp90 inhibitors, kinase inhibitors, MDM2 inhibitors, compounds targeting proteins containing human BET bromodomain, compounds targeting cytoplasmic signaling protein FKBP12, HDAC inhibitors, human lysine methyltransferase inhibitors, angiogenesis inhibitors, immunosuppressive compounds and compounds targeting aryl hydrocarbon receptor (AHR) and compounds targeting anaerobic tumor microenvironment.

In certain embodiments, the targeting ligand is capable of binding kinases, BET bromodomain-containing proteins, cytoplasmic signaling proteins (such as FKBP12), nucleoproteins, histone deacetylases, lysine methyl transferase, protein regulating angiogenesis, proteins regulating immune response, aromatic hydrocarbon receptors (AHRs), estrogen receptors, androgen receptors, glucocorticoid receptors, or transcription factor (e.g., SMARCA4, SMARCA2, TRIM24).

In certain embodiments, kinases, to which targeting ligands are capable of binding, include, but not limited to: Tyrosine kinases (for example, AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, HRAS, HSP90, IGF1R, ILK, INSR, INSRR, IRAK4, ITK, JAK1, JAK2, JAK3, KDR, KIT, K^{RA}S, KSP, KSR1, LCK, LMTK2, LMTK3, LTK, LYN, MATK, MERTK, MET, MLTK, MST1R, MUSK, NPR1, N^{RA}S, NTRK1, NTRK2, NTRK3, PDGF^{RA}, PDGF^{RB}, PLK4, PTK2, PTK2B, PTK6, PTK7, RET, ROR1, ROR2, ROS1, RYK, SGK493, SRC, SRMS, STYK1, SYK, TEC, TEK, TEX14, TIEl, TNK1, TNK2, TNNI3K, TXK, TYK2, TYRO3, YES1 or ZAP70), Serine/threonine kinase (such as Casein Kinase2, protein kinase A, protein kinase B, protein kinaseC, Raf kinase, CaM kinase, AKT1, AKT2, AKT3, ALK1, ALK2, ALK3, ALK4, AuroraA, AuroraB, AuroraC, CHK1, CHK2, CLK1, CLK2, CLK3, DAPK1, DAPK2, DAPK3, DMPK, ERK1, ERK2, ERK5, GCK, GSK3, HIPK, KHS1, LKB1, LOK, MAPKAPK2, MAPKAPK, MEK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIP5, RSK1, RSK2, SGK2, SGK3, SIK1, STK33, TAO1, TAO2, TGF-β, TLK2, TSSK1, TSSK2, MLK1 or MLK2), Cyclin-dependent kinases (such as Cdk1-Cdk11) and Leucine-rich repetitive kinase (such as LRRK2).

The main advantages of the present invention include:
(a) The compounds of the present invention have excellent effects in inducing the degradation of Cyclin K.
(b) In-depth research has found that the compounds of the present invention also have the function of inducing the degradation of other Cyclin, thereby further increasing cytotoxicity thereof.

The present invention was further elaborated hereafter in combination with specific embodiments. It should be understood that these examples are only used to illustrate the invention and not to limit the scope of the invention. The experimental methods without specific conditions in the following examples generally follow the conventional conditions or the conditions suggested by the manufacturer. Unless otherwise stated, percentages and parts are percentages by weight and parts by weight.

### A. Preparation Example

### General Synthesis Method

The compounds of the present invention may be prepared, isolated or obtained by any method that is obvious to a person skilled in the art. The compounds of the present invention may also be prepared according to the exemplary preparation schemes provided below (such as the methods in the Examples). Reaction conditions, steps and reactants not provided in the exemplary preparation schemes are obvious and known to a person skilled in the art. As used herein, the symbols and conventions used in these processes, schemes and examples, whether or not a particular abbreviation is specifically defined, have meanings that are well known to a person skilled in the art. Specifically, but not limited to, the following abbreviations may be used in the Examples and throughout the specification: g (gram); mg (milligram); mL (milliliter); μL (microliter); mm (millimole); μM (micromole); MHz (hertz); MHz (megahertz); mmol (millimole); hr or hrs (hours); min (minutes); MS (mass spectrometry); ESI (electrospray ionization); TLC (thin layer chromatography); HPLC (high performance liquid chromatography); THF (tetrahydrofuran); CDCl₃ (deuterated chloroform); AcOH (acetic acid); DCM (dichloromethane); DMSO (dimethyl sulfoxide); EtOAc (ethyl acetate); MeOH (methanol); and BOC (tert-butyloxycarbonyl), etc.

Unless otherwise stated, the starting materials used in the examples were obtained from commercial sources or synthesized in a manner known to those skilled in the art or in a manner analogous to that described in the examples.

### Example 1: Synthesis of Compound UB-001

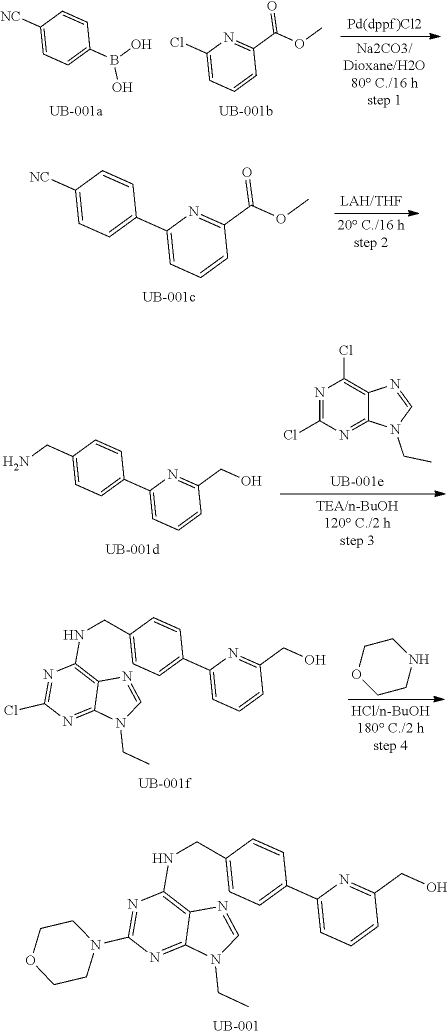

### Step 1: UB-001c

UB-001a (2000 mg, 13.6 mol), UB-001b (2.32 g, 13.6 mmol), pdCl₂dppf (300 mg), and Na₂CO₃ (2.8 g) were added to dioxane (32 mL) and water (8 mL). The reaction system was stirred at 80° C. for 16 hours, and cooled to room temperature after the completion of the reaction. The mixture was added into water, extracted with ethyl acetate, washed with brine (30 mL), dried over sodium sulfate, and filtered. After concentration, the residue was isolated by silica gel column chromatography (dichloromethane/methanol=5%) to obtain UB-001c as a yellow solid (1.8 g, yield 55.5%). LCMS [M+H]⁺=239.2

### Step 2: UB-001d

LAH (4.2 ml, 1M in THF) was added dropwise to UB-001c (500 mg, 2.1 mmol) in anhydrous THF (10 mL). The reaction system was stirred at 20° C. for 16 hours, and quenched with Na₂SO₄*10H₂O after the completion of the reaction. The mixture was added into water, extracted with ethyl acetate, washed with brine (30 mL), dried over sodium sulfate, and filtered. After concentration, the residue was isolated by silica gel column chromatography (dichloromethane/methanol=2-20%) to obtain UB-001d as a yellow solid (200 mg, yield 44.5%). LCMS [M+H]⁺=215.3

### Step 3: UB-001f

A mixture of UB-001d (100 mg, 0.47 mmol), and UB-001e (0.1 g, 0.47 mmol) was dissolved in n-butanol (2 mL) and reacted at 120° C. for 3 hours. The reaction solution was concentrated and the crude product was separated by column chromatography (PE/EA=30-70%) to give UB-001f (120 mg, 65.1% yield) as a white solid. LCMS [M+H]⁺=394.2

### Step 4: UB-001

A mixture of UB-001f (30 mg, 0.08 mmol), morpholine (33.12 mg, 0.38 mmol), and HCl (cat.) was dissolved in n-butanol (2 mL) and reacted at 180° C. for 5 hours. The reaction solution was concentrated and the crude product was separated by column chromatography (DCM/MeOH=0-10%) to give UB-001 (10 mg, 29.1% yield) as a white solid. 1H NMR (400 MHz, DMSO-d6) δ 8.06 (s, 1H), 8.03-7.95 (m, 2H), 7.89-7.73 (m, 3H), 7.45 (d, J=8.1 Hz, 2H), 7.40 (d, J=7.6 Hz, 1H), 5.43 (t, J=5.9 Hz, 1H), 4.65 (s, 1H), 4.61 (d, J=5.8 Hz, 2H), 4.02 (q, J=7.2 Hz, 2H), 3.61 (s, 8H), 1.35 (t, J=7.2 Hz, 3H). LCMS [M+H]⁺=446.6

### Example 2: Synthesis of Compound UB-002

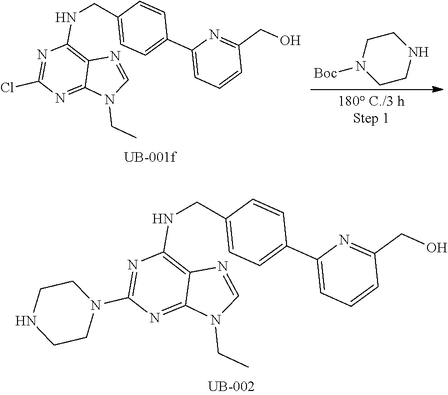 [SMILES: CCn1cnc2c(NCc3ccc(-c4cccc(CO)n4)cc3)nc(Cl)nc21 ; CC(C)(C)OC(=O)N1CCNCC1 ; CCn1cnc2c(NCc3ccc(-c4cccc(CO)n4)cc3)nc(N3CCNCC3)nc21]

### Step 1: UB-002

A mixture of UB-001f (30 mg, 0.08 mmol), 1-Boc-piperazine (14 mg, 0.08 mmol), and HCl (cat.) was dissolved in n-butanol (2 mL) and reacted at 180° C. for 3 hours. The reaction solution was concentrated and the crude product was separated by column chromatography (DCM/MeOH=0-10%) to give a yellow solid (5 mg, 14.8% yield). ¹H NMR (400 MHz, DMSO-d6) δ 8.16 (s, 1H), 7.99 (d, J=8.0 Hz, 2H), 7.88-7.81 (m, 2H), 7.76 (d, J=7.8 Hz, 1H), 7.46 (d, J=8.0 Hz, 2H), 7.41 (d, J=7.6 Hz, 1H), 5.45 (s, 1H), 4.67-4.58 (m, 3H), 4.03 (q, J=7.2 Hz, 2H), 3.80 (t, J=5.2 Hz, 4H), 2.96 (t, J=5.1 Hz, 4H), 1.36 (t, J=7.2 Hz, 3H), 1.24 (d, J=3.5 Hz, 1H). LCMS [M+H]⁺=445.6

### Example 3: Synthesis of Compound UB-003

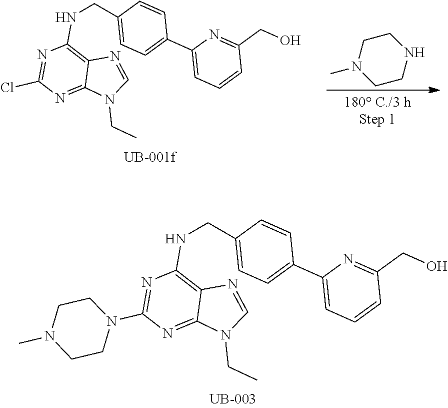

### Step 1: UB-003

A mixture of UB-001f (30 mg, 0.08 mmol), 1-methyl-piperazine (76 mg, 0.8 mmol), and HCl (cat.) was dissolved in n-butanol (2 mL) and reacted at 180° C. for 3 hours. The reaction solution was concentrated and the crude product was separated by preparative TLC plate (DCM/MeOH=10%) to give UB-003 (5.9 mg, 16.9% yield) as a yellow solid. ¹H NMR (400 MHz, DMSO-d6) δ 8.05 (s, 1H), 8.01-7.96 (m, 2H), 7.87-7.79 (m, 2H), 7.75 (d, J=7.8 Hz, 1H), 7.45 (d, J=8.1 Hz, 2H), 7.40 (d, J=7.5 Hz, 1H), 5.45 (t, J=5.9 Hz, 1H), 4.62 (t, J=8.0 Hz, 3H), 4.02 (q, J=7.2 Hz, 2H), 3.70 (s, 4H), 2.49-2.39 (m, 4H), 2.24 (s, 3H), 1.35 (t, J=7.2 Hz, 3H). LCMS [M+H]⁺=459.3.

### Example 4: Synthesis of Compound UB-004

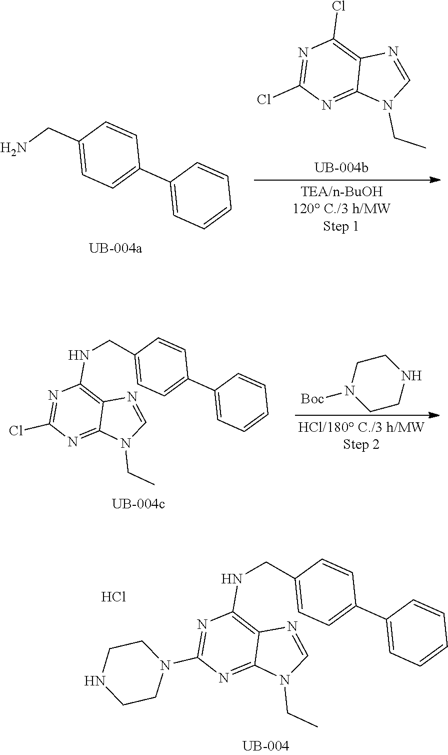 [SMILES: NCc1ccc(-c2ccccc2)cc1 ; CCn1cnc2c(Cl)nc(Cl)nc21 ; CCn1cnc2c(NCc3ccc(-c4ccccc4)cc3)nc(Cl)nc21 ; CC(C)(C)OC(=O)N1CCNCC1 ; CCn1cnc2c(NCc3ccc(-c4ccccc4)cc3)nc(N3CCNCC3)nc21 ; Cl]

### Step 1: UB-004 cP

A mixture of UB-004a (100 mg, 0.47 mmol), UB-004b (101 mg, 0.47 mmol), and DIPEA (95 mg) was dissolved in n-butanol (2 mL) and reacted at 120° C. for 3 hours. The reaction solution was concentrated and the crude product was separated by column chromatography (PE/EA=30-70%) to give UB-004c (120 mg, 60.4% yield) as a white solid. LCMS [M+H]+=364.2

### Step 2: UB-004

A mixture of UB-004e (30 mg, 0.08 mmol), tert-butyl piperazine-1-carboxylate (70.8 mg, 0.38 mmol), and HCl (cat.) was dissolved in n-butanol (2 mL) and reacted at 180° C. for 5 hours. The reaction solution was concentrated and the crude product was separated by column chromatography (DCM/MeOH=0-10%) to give UB-004 (10 mg, 27% yield) as a yellow solid. ¹H NMR (400 MHz, DMSO-d6) δ 7.78 (s, 1H), 7.65-7.54 (m, 4H), 7.48-7.40 (m, 4H), 7.37-7.30 (m, 1H), 4.63 (s, 2H), 4.01 (q, J=7.2 Hz, 2H), 3.68-3.54 (m, 4H), 2.75-2.63 (m, 4H), 1.35 (t, J=7.2 Hz, 3H). LCMS [M+H]⁺=414.4

### Example 5: Synthesis of Compound UB-005

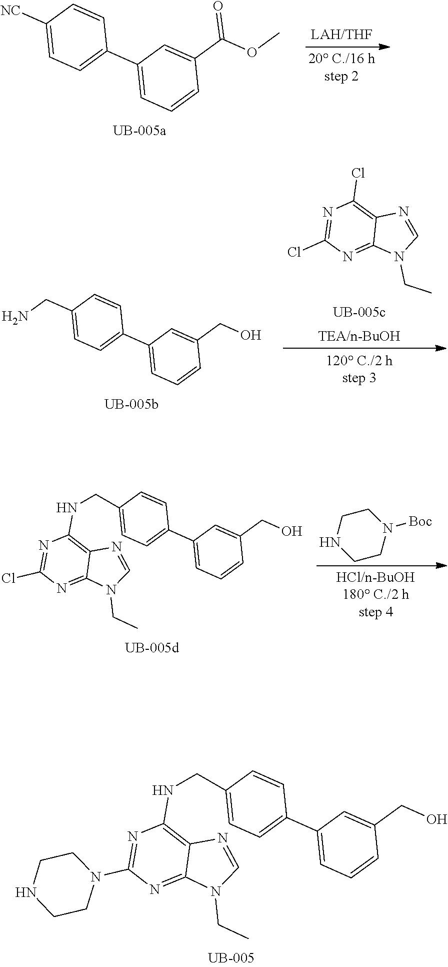 [SMILES: COC(=O)c1cccc(-c2ccc(C#N)cc2)c1 ; CCn1cnc2c(Cl)nc(Cl)nc21 ; NCc1ccc(-c2cccc(CO)c2)cc1 ; CCn1cnc2c(NCc3ccc(-c4cccc(CO)c4)cc3)nc(Cl)nc21 ; CC(C)(C)OC(=O)N1CCNCC1 ; CCn1cnc2c(NCc3ccc(-c4cccc(CO)c4)cc3)nc(N3CCNCC3)nc21]

### Step 1: UB-005a

LAH (4.2 ml, 1M in THF) was added dropwise to UB-005a (500 mg, 2.1 mmol) in anhydrous THF (10 mL). The reaction system was stirred at 20° C. for 16 hours, and quenched with Na₂SO₄*10H₂O after the completion of the reaction. The mixture was added into water, extracted with ethyl acetate, washed with brine (30 mL), dried over sodium sulfate, and filtered. After concentration, the residue was isolated by silica gel column chromatography (dichloromethane/methanol=2-20%) to obtain UB-005b (200 mg, yield 44.5%) as a yellow solid. LCMS [M+H]⁺=214.2

### Step 2: UB-005d

A mixture of UB-005b (100 mg, 0.47 mmol), UB-005c (101 mg, 0.47 mmol), and DIPEA (95 mg) was dissolved in n-butanol (2 mL) and reacted at 120° C. for 3 hours. The reaction solution was concentrated and the crude product was separated by column chromatography (PE/EA=30-70%) to give UB-005d (120 mg, 65.1% yield) as a white solid. LCMS [M+H]=394.3

### Step 3: UB-005

A mixture of UB-005e (30 mg, 0.08 mmol), tert-butyl piperazine-1-carboxylate (70.8 mg, 0.38 mmol), and HCl (cat.) was dissolved in n-butanol (2 mL) and reacted at 180° C. for 5 hours. The reaction solution was concentrated and the crude product was separated by column chromatography (DCM/MeOH=0-10%) to give UB-005 (10 mg, 29.5% yield) as a yellow solid. H NMR (400 MHz, DMSO-d3) δ 7.99 (d, J=8.3 Hz, 3H), 7.87-7.73 (m, 3H), 7.45 (d, J=8.1 Hz, 2H), 7.40 (d, J=7.6 Hz, 1H), 5.41 (s, 1H), 4.61 (d, J=4.2 Hz, 4H), 4.01 (q, J=7.2 Hz, 2H), 3.63 (t, J=5.1 Hz, 4H), 2.74 (d, J=10.2 Hz, 4H), 1.35 (t, J=7.2 Hz, 3H). LCMS [M+H]⁺=444.6

### Example 6: Synthesis of Compound UB-006

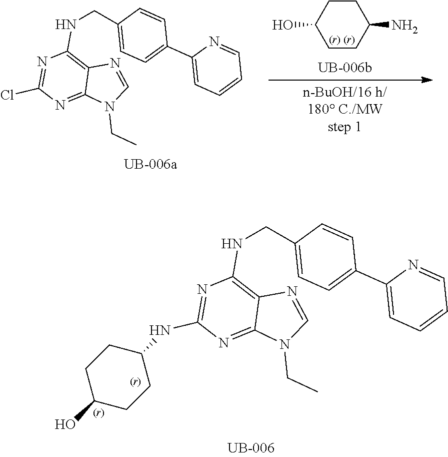 [SMILES: CCn1cnc2c(NCc3ccc(-c4ccccn4)cc3)nc(Cl)nc21 ; N[C@H]1CC[C@H](O)CC1 ; CCn1cnc2c(NCc3ccc(-c4ccccn4)cc3)nc(N[C@H]3CC[C@H](O)CC3)nc21]

### Step 1: UB-006

A mixture of UB-006a (50 mg, 0.14 mmol), and UB-006b (59.7 mg, 0.69 mmol) was dissolved in n-butanol (2 mL) and reacted at 180° C. for 16 hours. The reaction solution was concentrated and the crude product was separated by column chromatography (DCM/MeOH=0-10%) to give UB-006 (16 mg, 26.3% yield) as a yellow solid. ¹H NMR (400 MHz, DMSO-d6) δ 8.67-8.60 (m, 1H), 8.04-7.97 (m, 2H), 7.93-7.82 (m, 2H), 7.72 (s, 1H), 7.45 (d, J=8.0 Hz, 2H), 7.35-7.28 (m, 1H), 6.08 (d, J=7.6 Hz, 1H), 4.65 (s, 2H), 4.48 (d, J=4.5 Hz, 1H), 3.97 (q, J=7.2 Hz, 2H), 3.61 (s, 1H), 1.79 (d, J=11.3 Hz, 4H), 1.33 (t, J=7.2 Hz, 3H), 1.28-1.14 (m, 5H). LCMS [M+H]⁺=444.9

### Example 7: Synthesis Method of Compound UB-007

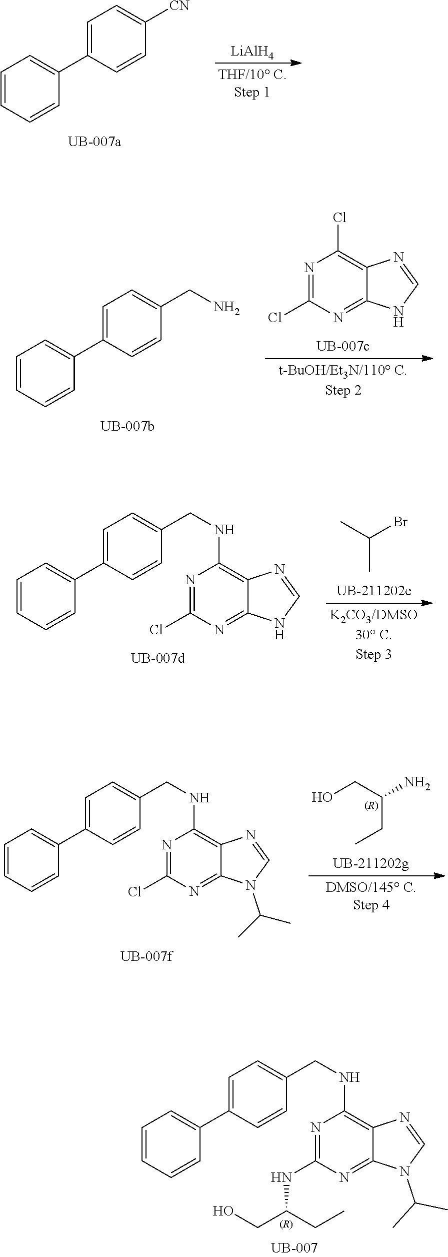

### Step 1: UB-007b

Compound UB-007a (35.8 g, 0.2 mol) was added to a three-necked reaction flask and dissolved with tetrahydrofuran (360 mL). Under argon protection, lithium aluminum tetrahydride (17.6 g, 0.44 mol) was added in five batches. The reaction solution was reacted at 16° C. for 2 hours, the reaction solution was quenched with saturated sodium sulfate solution (20 g), the solid was filtered, and the filter cake was washed with tetrahydrofuran (100 mL).

The filtrate was dried by rotary evaporation to obtain the product (UB-007b, 37 g, 100% yield) as a yellow solid. LCMS [M+1]⁺=184.3

### Step 2: UB-007d

Compound UB-007b (18.4 g, 0.1 mol), UB-007c (18.9 g, 0.1 mol), and triethylamine (40.4 g, 0.4 mol) were dissolved in tert-butanol (200 mL), and reacted at 110° C. for 14 hours.

The reaction solution was cooled to 20° C., and a large amount of solid precipitated. The solid was filtered and the filter cake was washed with ethanol/water (100 mL/10 mL) and dried in vacuo to obtain the target compound (UB-007d, 20.7 g, 80% yield) as a yellow solid. LCMS [M+1]⁺=336.8

### Step 3: UB-007f

Compound UB-007d (27 g, 0.08 mol), and K₂CO₃ (55.2 g, 0.4 mol) were dissolved in dimethyl sulfoxide (270 mL), and the resulting mixture was stirred at room temperature for 5 min. Compound UB-007e (59.3 g, 0.48 mol) was added dropwise to the reaction solution. After the dropwise addition was completed, the reaction solution was reacted at 30° C. for 14 hours.

The reaction solution was quenched by adding water (1000 mL) and extracted with ethyl acetate (200 mL*3). The organic layer was washed with brine, dried over anhydrous sodium sulfate, and spin-dried. The residue was separated and purified by column chromatography (petroleum ether/ethyl acetate=5/1) to obtain the compound (UB-007f, 27 g, 90% yield) as a light yellow solid. LCMS [M+1]⁺=378.9. 1H NMR (400 MHz, CDCl3) δ 7.72 (s, 1H), 7.62 (dd, J=8.0, 2.0 Hz, 4H), 7.48 (t, J=8.1 Hz, 4H), 7.39 (t, J=7.3 Hz, 1H), 6.58 (s, 1H), 5.04-4.80 (m, 3H), 1.60 (d, J=6.8 Hz, 6H).

### Step 4: UB-007

Compound UB-007f (110 mg, 0.29 mmol), and compound UB-007g (446 mg, 1.45 mmol) were dissolved in dimethyl sulfoxide (2 mL), and reacted at 145° C. for 14 hours. The reaction solution was cooled to 20° C., and extracted with ethyl acetate (20 mL) three times. The organic layer was washed with brine, dried over anhydrous sodium sulfate, and spin-dried. The residue was separated and purified by column chromatography (petroleum ether/ethyl acetate=1/2) to obtain compound (UB-007, 39 mg, 31.2% yield) as a light yellow solid. LCMS [M+1]⁺=431.6. ¹H NMR (400 MHz, CDCl₃) δ 7.66-7.58 (m, 4H), 7.57 (s, 1H), 7.53-7.44 (m, 4H), 7.38 (t, J=7.3 Hz, 1H), 6.77 (s, 1H), 5.00 (d, J=6.0 Hz, 1H), 4.83 (s, 2H), 4.61 (dt, J=13.5, 6.8 Hz, 1H), 4.01-3.91 (m, 1H), 3.85 (dd, J=10.7, 2.3 Hz, 1H), 3.67 (dd, J=10.5, 7.6 Hz, 1H), 1.73-1.57 (m, 2H), 1.53 (d, J=6.7 Hz, 6H), 1.05 (t, J=7.4 Hz, 3H).

### Example 8: Synthesis Method of Compound UB-008

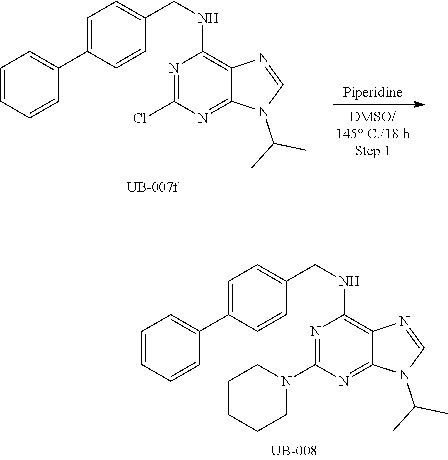 [SMILES: CC(C)n1cnc2c(NCc3ccc(-c4ccccc4)cc3)nc(Cl)nc21 ; CC(C)n1cnc2c(NCc3ccc(-c4ccccc4)cc3)nc(N3CCCCC3)nc21]

### Step 1: UB-008 (LS22002-016)

Compound UB-007f (185.15 mg, 0.49 mmol), and piperidine (200 mg, 2.35 mmol) were dissolved in dimethyl sulfoxide (2 mL), and reacted at 145° C. for 18 hours. The reaction solution was cooled to 20° C., quenched by adding water (50 mL) and extracted with ethyl acetate (20 mL*3). The organic layer was washed with brine, dried over anhydrous sodium sulfate, and spin-dried. The residue was separated and purified by column chromatography (petroleum ether/ethyl acetate=1/2) to obtain the compound (UB-007, 31 mg, 14.8% yield) as a light yellow solid. LCMS [M+1]=427.5

### Example 9: Synthesis of Compound UB-009

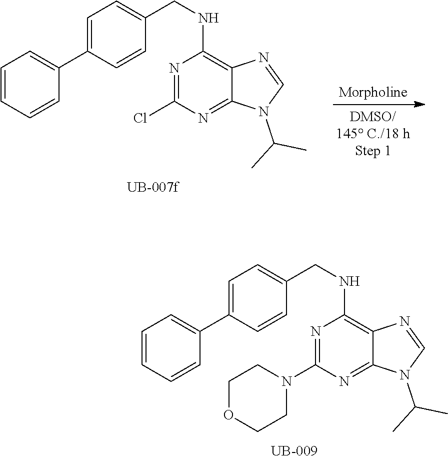 [SMILES: CC(C)n1cnc2c(NCc3ccc(-c4ccccc4)cc3)nc(Cl)nc21 ; CC(C)n1cnc2c(NCc3ccc(-c4ccccc4)cc3)nc(N3CCOCC3)nc21]

### Step 1: UB-009

Compound UB-007f (200 mg, 0.53 mmol), and morpholine (416 mg, 5.3 mmol) were dissolved in dimethyl sulfoxide (3 mL), and reacted at 145° C. for 18 hours. The reaction solution was cooled to 20° C., quenched by water (50 mL) and extracted with ethyl acetate (20 mL*3). The organic layer was washed with brine, dried over anhydrous sodium sulfate, and spin-dried. The residue was separated and purified by column chromatography (petroleum ether/ethyl acetate=1/2) to obtain the compound (UB-009, 38.5 mg, 17% yield) as a light yellow solid. LCMS [M+1]⁺=429.6. ¹H NMR (400 MHz, CDCl₃) δ 7.68 (s, 1H), 7.59 (dd, J=15.8, 7.8 Hz, 4H), 7.51-7.43 (m, 4H), 7.38 (t, J=7.3 Hz, 1H), 6.64 (s, 1H), 4.85 (s, 2H), 4.75-4.60 (m, 1H), 3.80 (d, J=3.4 Hz, 8H), 1.56 (d, J=6.7 Hz, 6H).

### Example 10: Synthesis of Compound UB-010

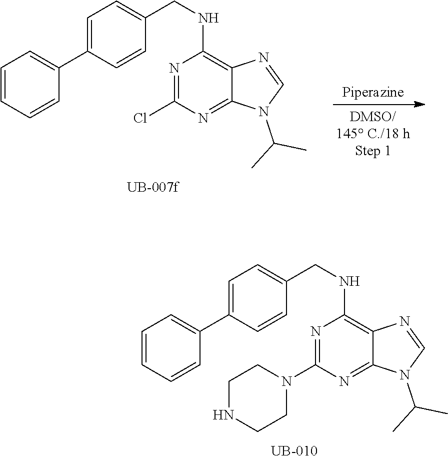 [SMILES: CC(C)n1cnc2c(NCc3ccc(-c4ccccc4)cc3)nc(Cl)nc21 ; CC(C)n1cnc2c(NCc3ccc(-c4ccccc4)cc3)nc(N3CCNCC3)nc21]

### Step 1: UB-010

Compound UB-007f (200 mg, 0.53 mmol), and piperazine (461 mg, 5.3 mmol) were dissolved in dimethyl sulfoxide (3 mL), and reacted at 145° C. for 18 hours. The reaction solution was cooled to 20° C., quenched by adding water (20 mL) and extracted with ethyl acetate (20 mL*3). The organic layer was washed with brine, dried over anhydrous sodium sulfate, and spin-dried. The residue was separated and purified by column chromatography (dichloromethane/methanol=13/1) to obtain the compound (UB-010, 85 mg, 37.5% yield) as a light yellow solid. LCMS [M+1]⁺=428.6. ¹H NMR (400 MHz, CDCl₃) δ 7.80 (s, 1H), 7.57 (dd, J=12.3, 7.9 Hz, 4H), 7.45 (dd, J=12.5, 7.7 Hz, 4H), 7.36 (s, 1H), 4.79 (s, 2H), 4.66 (dt, J=12.9, 6.3 Hz, 2H), 4.11 (s, 4H), 3.22 (s, 4H), 1.55 (d, J=6.7 Hz, 6H).

### Example 11: Synthesis of Compound UB-011

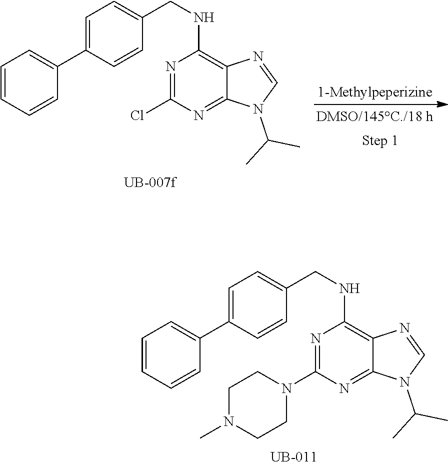 [SMILES: CC(C)n1cnc2c(NCc3ccc(-c4ccccc4)cc3)nc(Cl)nc21 ; CC(C)n1cnc2c(NCc3ccc(-c4ccccc4)cc3)nc(N3CCN(C)CC3)nc21]

### Step 1: UB-011

Compound UB-007f (110 mg, 0.29 mmol), and N-methyl piperazine (290 mg, 2.9 mmol) were dissolved in dimethyl sulfoxide (2 mL), and reacted at 145° C. for 18 hours. The reaction solution was cooled to 20° C., quenched by adding water (20 mL) and extracted with ethyl acetate (20 mL*3). The organic layer was washed with brine, dried over anhydrous sodium sulfate, and spin-dried. The residue was separated and purified by column chromatography (dichloromethane/methanol=13/1) to obtain the compound (UB-011, 53.9 mg, 42% yield) as a light yellow solid. LCMS [M+1]⁺=442.7. ¹H NMR (400 MHz, CDCl₃) δ 7.59 (dd, J=12.8, 7.9 Hz, 5H), 7.50-7.44 (m, 4H), 7.37 (t, J=7.3 Hz, 1H), 6.43 (s, 1H), 4.85 (s, 2H), 4.73-4.63 (m, 1H), 4.00 (s, 4H), 2.67 (s, 4H), 2.49 (s, 3H), 1.56 (d, J=6.8 Hz, 6H).

### Example 12: Synthesis of Compound UB-012

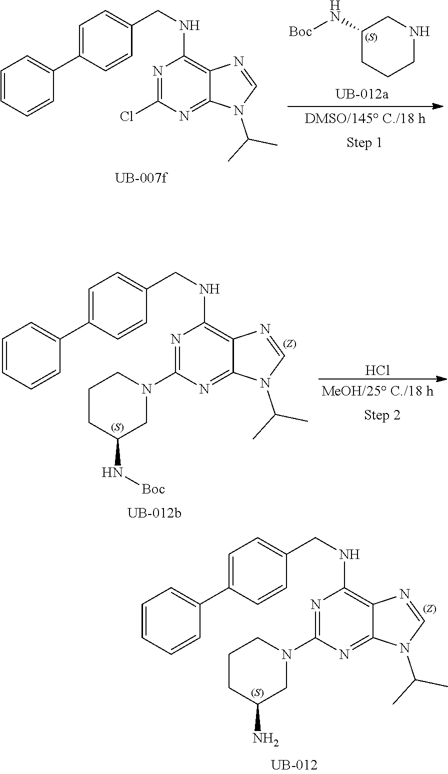

### Step 1: UB-012b

Compound UB-007f (188.5 mg, 0.5 mmol), and compound UB-012a (500 mg, 2.5 mmol) were dissolved in dimethyl sulfoxide (3 mL), and reacted at 145° C. for 18 hours. The reaction solution was cooled to 20° C., quenched by adding water (20 mL) and extracted with ethyl acetate (20 mL*3). The organic layer was washed with brine, dried over anhydrous sodium sulfate, and spin-dried to obtain the compound (UB-012b, 200 mg, 90.1% yield) as a light yellow solid. LCMS [M+1]⁺=542.6

### Step 2: UB-012

Compound UB-012b (200 mg, 0.45 mmol) was dissolved in methanol (3 mL), 1N hydrochloric acid methanol gas (3 mL) was added under stirring, and the reaction was carried out at 25° C. for 18 hours. The reaction solution was spin-dried, the pH was adjusted to 9 with saturated sodium carbonate solution, and extracted with ethyl acetate (20 mL*3). The organic layer was washed with brine, dried over anhydrous sodium sulfate, and spin-dried. The residue was separated and purified by column chromatography (dichloromethane/methanol=13/1) to obtain the compound (UB-012, 131 mg, 66% yield) as a light yellow solid. LCMS [M+1]⁺=442.7. ¹H NMR (500 MHz, DMSO-d6) δ 8.17 (s, 2H), 7.81 (s, 1H), 7.61 (dd, J=15.5, 7.7 Hz, 4H), 7.44 (t, J=7.8 Hz, 4H), 7.34 (t, J=7.3 Hz, 1H), 6.17 (s, 1H), 4.63 (s, 2H), 4.57-4.44 (m, 1H), 3.61 (d, J=7.2 Hz, 1H), 2.91 (t, J=11.1 Hz, 1H), 1.95 (t, J=15.9 Hz, 4H), 1.45 (t, J=12.4 Hz, 7H), 1.32-1.07 (m, 2H).

### Example 13: Synthesis of Compound UB-013

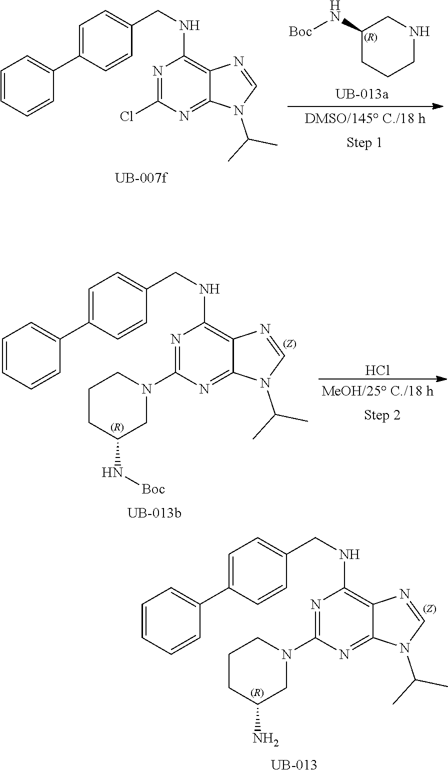

### Step 1: UB-013b (LS22002-011-1)

Compound UB-007f (188.5 mg, 0.5 mmol), and compound UB-012a (500 mg, 2.5 mmol) were dissolved in dimethyl sulfoxide (3 mL), and reacted at 145° C. for 18 hours. The reaction solution was cooled to 20° C., quenched by adding water (20 mL) and extracted with ethyl acetate (20 mL*3). The organic layer was washed with brine, dried over anhydrous sodium sulfate, and spin-dried to obtain the compound (UB-013b, 200 mg, 90.1% yield) as a light yellow solid. LCMS [M+1]=542.6

### Step 2: UB-013 (LS22002-011-2)

Compound UB-013b (200 mg, 0.45 mmol) was dissolved in methanol (3 mL), 1N hydrochloric acid methanol gas (3 mL) was added under stirring, and the reaction was carried out at 25° C. for 18 hours. The reaction solution was spin-dried, the pH was adjusted to 9 with saturated sodium carbonate solution, and extracted with ethyl acetate (20 mL*3). The organic layer was washed with brine, dried over anhydrous sodium sulfate, and spin-dried. The residue was separated and purified by column chromatography (dichloromethane/methanol=13/1) to obtain the compound (UB-013, 77 mg, 38% yield) as a light yellow solid. LCMS [M+1]⁺=442.7 ¹H NMR (500 MHz, DMSO-d6) δ 8.09 (s, 1H), 7.89 (s, 1H), 7.61 (dd, J=18.2, 7.8 Hz, 4H), 7.50 (d, J=7.7 Hz, 2H), 7.44 (t, J=7.7 Hz, 2H), 7.34 (t, J=7.3 Hz, 1H), 4.75-4.56 (m, 3H), 4.30 (d, J=12.9 Hz, 1H), 3.54-3.21 (m, 2H), 3.09 (dd, J=13.9, 6.2 Hz, 2H), 3.00 (t, J=10.9 Hz, 1H), 2.51 (s, 1H), 2.01 (d, J=10.0 Hz, 1H), 1.73 (dd, J=9.2, 4.0 Hz, 1H), 1.63-1.52 (m, 1H), 1.47 (t, J=10.0 Hz, 7H).

### Example 14: Synthesis of Compound UB-014

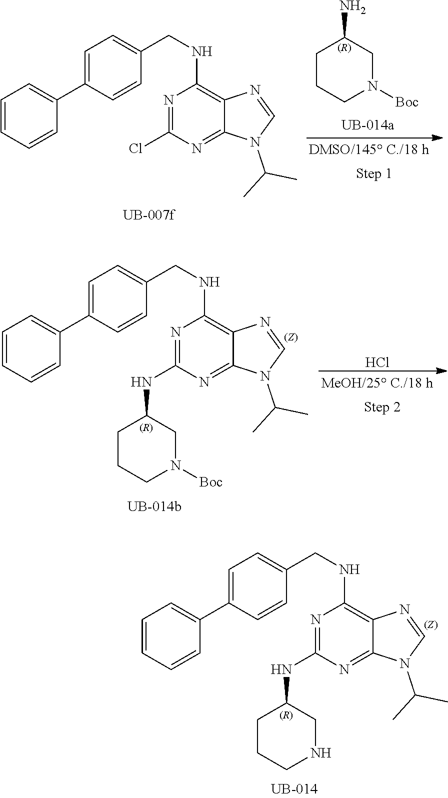

### Step 1: UB-014b

Compound UB-007f (188.5 mg, 0.5 mmol), and compound UB-014a (500 mg, 2.5 mmol) were dissolved in dimethyl sulfoxide (3 mL), and reacted at 145° C. for 18 hours. The reaction solution was cooled to 20° C., quenched by adding water (20 mL) and extracted with ethyl acetate (20 mL*3). The organic layer was washed with brine, dried over anhydrous sodium sulfate, and spin-dried to obtain the compound (UB-014b, 200 mg, 90.1% yield) as a light yellow solid. The crude product was directly used in the next step. LCMS [M+1]=542.6

### Step 2: UB-014

Compound UB-014b (200 mg, 0.45 mmol) was dissolved in methanol (3 mL), 1N hydrochloric acid methanol gas (3 mL) was added under stirring, and the reaction was carried out at 25° C. for 18 hours. The reaction solution was spin-dried, the pH of which was adjusted to 9 with saturated sodium carbonate solution, and extracted with ethyl acetate (20 mL*3). The organic layer was washed with brine, dried over anhydrous sodium sulfate, and spin-dried. The residue was separated and purified by column chromatography (dichloromethane/methanol=13/1) to obtain the compound (UB-014, 35 mg, 17.6% yield) as a light yellow solid. LCMS [M+1]⁺=442.5. H NMR (400 MHz, DMSO-d₆) δ 8.05 (s, 1H), 7.90 (s, 1H), 7.63 (dd, J=14.2, 8.0 Hz, 4H), 7.48 (dd, J=16.7, 8.5 Hz, 4H), 7.37 (t, J=7.3 Hz, 1H), 4.80-4.47 (m, 4H), 4.31 (d, J=12.9 Hz, ¹H), 3.03 (d, J=7.5 Hz, 4H), 2.02-1.92 (m, 1H), 1.79-1.69 (m, 1H), 1.51 (d, J=6.8 Hz, 9H).

### Example 15: Synthesis of Compound UB-015

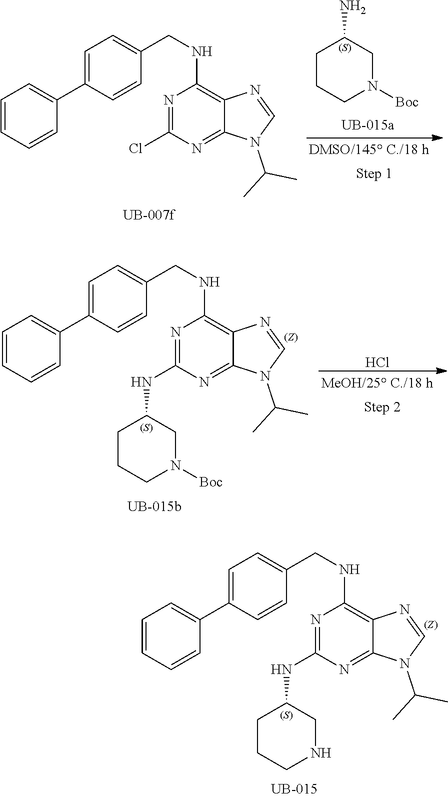

### Step 1: UB-015b

Compound UB-007f (188.5 mg, 0.5 mmol), and compound UB-015a (500 mg, 2.5 mmol) were dissolved in dimethyl sulfoxide (3 mL), and reacted at 145° C. for 18 hours. The reaction solution was cooled to 20° C., quenched by adding water (20 mL) and extracted with ethyl acetate (20 mL*3). The organic layer was washed with brine, dried over anhydrous sodium sulfate, and spin-dried to obtain the compound (UB-015b, 200 mg, 90.1% yield) as a light yellow solid. The crude product was directly used in the next step. LCMS [M+1]=542.5

### Step 2: UB-015

Compound UB-015b (200 mg, 0.45 mmol) was dissolved in methanol (3 mL), 1N hydrochloric acid methanol gas (3 mL) was added under stirring, and the reaction was carried out at 25° C. for 18 hours. The reaction solution was spin-dried, the pH of which was adjusted to 9 with saturated potassium carbonate solution, and extracted with ethyl acetate (20 mL*3). The organic layer was washed with brine, dried over anhydrous sodium sulfate, and spin-dried. The residue was separated and purified by column chromatography (dichloromethane/methanol=13/1) to obtain the compound (UB-015, 45 mg, 22.6% yield) as a light yellow solid. LCMS [M+1]=442.5

### Example 16: Synthesis of Compound UB-016

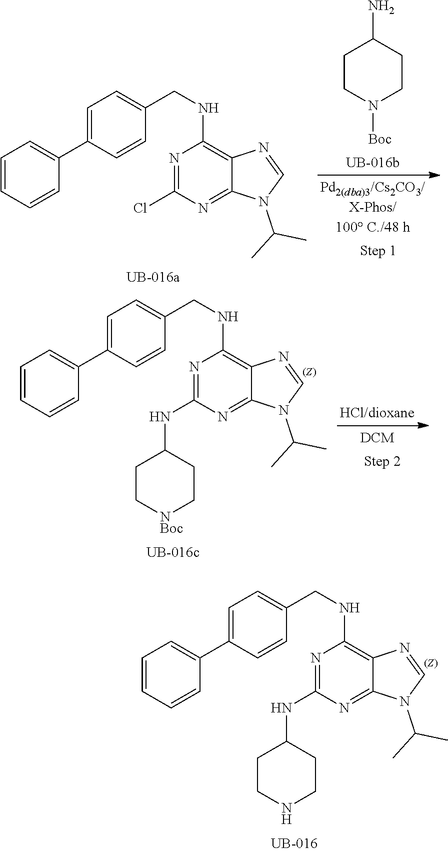

### Step 1: UB-016c

N⁶—([1,1′-biphenyl]-4-ylmethyl)-9-isopropyl-N²-(piperidin-4-yl)-9H-purin-2,6-diamine UB-016a (200 mg, 0.53 mmol), UB-016b (318.4 mg, 1.59 mmol), Pd₂(dba)₃ (49 mg, 0.054 mmol), Xphos (126 mg, 0.265 mmol) and Cs2CO₃ (518 mg, 1.59 mmol), and 1,4-dioxane (10 mL) were added to a reaction flask. The reaction mixture was replaced with argon three times and then heated to 100° C. and stirred overnight. The reaction mixture was diluted with water (30 mL), and then extracted with ethyl acetate (30 mL*3). The organic phase was separated, washed with brine (50 mL) and dried over anhydrous sodium sulfate, then concentrated and purified by chromatography (dichloromethane:dichloromethane/methanol 10/1=40:60) to obtain a yellow solid compound (UB-016c, 369.6 mg, crude product) LCMS [M+1]⁺=542.3.

### Step 2: UB-016

### N⁶—([1,1′-biphenyl]-4-ylmethyl)-9-isopropyl-N²-(piperidin-4-yl)-9H-purin-2,6-diamine

To a 100 mL bottom flask were added UB-016c (369.6 mg, 0.68 mmol), and a solution of hydrochloric acid in 1,4-dioxane (4 mL), and dichloromethane (8.0 mL) at room temperature. Then the solution was stirred at room temperature for 2 hours. The reaction mixture was filtered; the filter cake was washed with dichloromethane (5 mL), neutralized with saturated aqueous sodium bicarbonate solution, and extracted with dichloromethane (3*50 mL). The combined organic phase was washed with brine, dried over anhydrous sodium sulfate and concentrated under reduced pressure, then added with methanol and water and freeze-dried to obtain a yellow solid product (UB-016, 85.6 mg, yield: 28.4%) LCMS [M+1]⁺=442.2. ¹H NMR (400 MHz, DMSO-d6) δ 7.80 (s, 1H), 7.59 (dd, J=17.4, 7.8 Hz, 4H), 7.47-7.40 (m, 4H), 7.33 (t, J=7.3 Hz, 1H), 6.17 (d, J=7.2 Hz, 1H), 4.64 (s, 2H), 4.52 (dt, J=13.2, 6.5 Hz, 1H), 3.72 (s, 1H), 2.95 (d, J=11.3 Hz, 2H), 2.56 (d, J=11.2 Hz, 1H), 1.86 (s, 2H), 1.78 (s, 2H), 1.46 (d, J=6.7 Hz, 6H), 1.32 (dd, J=16.4, 8.8 Hz, 2H), 1.24 (d, J=6.1 Hz, 1H).

### Example 17: Synthesis of Compound UB-017

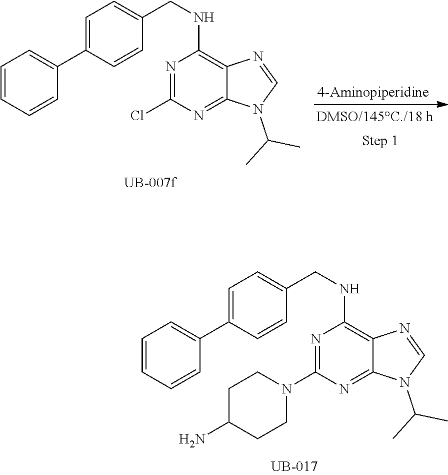 [SMILES: CC(C)n1cnc2c(NCc3ccc(-c4ccccc4)cc3)nc(Cl)nc21 ; CC(C)n1cnc2c(NCc3ccc(-c4ccccc4)cc3)nc(N3CCC(N)CC3)nc21]

### Step 1: UB-017

Compound UB-007f (188.5 mg, 0.5 mmol), and 4-aminopiperidine (250 mg, 2.5 mmol) were dissolved in dimethyl sulfoxide (3 mL), and reacted at 145° C. for 18 hours. The reaction solution was cooled to 20° C., quenched by adding water (20 mL) and extracted with ethyl acetate (20 mL*3). The organic layer was washed with brine, dried over anhydrous sodium sulfate, and spin-dried. The residue was separated and purified by column chromatography (petroleum ether/ethyl acetate=1/2) to obtain the compound (UB-017, 147 mg, 66% yield) as a light yellow solid. LCMS [M+1]⁺=442.7. ¹H NMR (500 MHz, DMSO-d6) δ 8.09 (s, 1H), 7.87 (s, 1H), 7.78 (s, 2H), 7.65-7.60 (m, 2H), 7.58 (d, J=8.2 Hz, 2H), 7.48-7.42 (m, 4H), 7.34 (t, J=7.4 Hz, 1H), 4.68-4.54 (m, 4H), 3.26-3.16 (m, 1H), 2.86 (t, J=11.7 Hz, 2H), 2.54 (s, 1H), 1.88 (d, J=10.5 Hz, 2H), 1.47 (d, J=6.8 Hz, 6H), 1.37 (dd, J=19.5, 10.2 Hz, 2H).

### Example 18: Synthesis of Compound UB-018

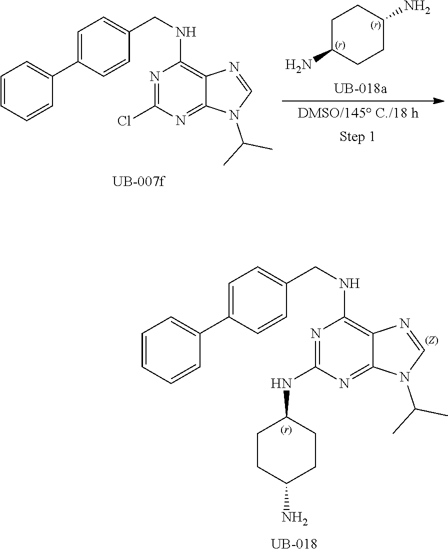 [SMILES: CC(C)n1cnc2c(NCc3ccc(-c4ccccc4)cc3)nc(Cl)nc21 ; N[C@H]1CC[C@H](N)CC1 ; CC(C)n1cnc2c(NCc3ccc(-c4ccccc4)cc3)nc(N[C@H]3CC[C@H](N)CC3)nc21]

### Step 1: UB-018

Compound UB-007f (188.5 mg, 0.5 mmol), and compound UB-018a (285.5 mg, 2.5 mmol) were dissolved in dimethyl sulfoxide (3 mL), and reacted at 145° C. for 14 hours. The reaction solution was cooled to 20° C., quenched by adding water (20 mL) and extracted with ethyl acetate (20 mL*3). The organic layer was washed with brine, dried over anhydrous sodium sulfate, and spin-dried. The residue was separated and purified by column chromatography (petroleum ether/ethyl acetate=1/2) to obtain the compound (UB-018, 85 mg, 37% yield) as a light yellow solid. LCMS [M+1]=456.3 ¹H NMR (500 MHz, DMSO-d6) δ 8.08 (s, 1H), 7.89 (s, 2H), 7.62 (d, J=7.3 Hz, 2H), 7.59 (d, J=8.2 Hz, 2H), 7.48 (d, J=7.9 Hz, 2H), 7.44 (t, J=7.7 Hz, 2H), 7.34 (t, J=7.3 Hz, 1H), 4.79-4.49 (m, 4H), 4.27 (d, J=13.1 Hz, 1H), 3.15-2.97 (m, 3H), 2.54 (s, 1H), 2.05-1.94 (m, 1H), 1.73 (dd, J=8.9, 4.4 Hz, 1H), 1.54 (d, J=8.7 Hz, 1H), 1.48 (d, J=6.8 Hz, 8H).

### Example 19: Synthesis of Compound UB-019

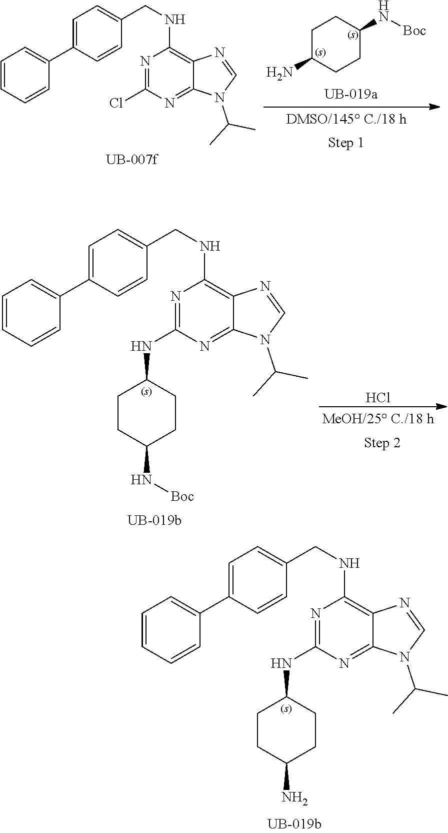 [SMILES: CC(C)n1cnc2c(NCc3ccc(-c4ccccc4)cc3)nc(Cl)nc21 ; CC(C)(C)OC(=O)N[C@H]1CC[C@@H](N)CC1 ; CC(C)n1cnc2c(NCc3ccc(-c4ccccc4)cc3)nc(N[C@H]3CC[C@@H](NC(=O)OC(C)(C)C)CC3)nc21 ; Cl ; CC(C)n1cnc2c(NCc3ccc(-c4ccccc4)cc3)nc(N[C@H]3CC[C@@H](N)CC3)nc21]

### Step 1: UB-019b

Compound UB-007f (188.5 mg, 0.5 mmol), and compound UB-019a (535.5 mg, 2.5 mmol) were dissolved in dimethyl sulfoxide (3 mL), and reacted at 145° C. for 14 hours. The reaction solution was cooled to 20° C., quenched by adding water (20 mL) and extracted with ethyl acetate (20 mL*3). The organic layer was washed with brine, dried over anhydrous sodium sulfate, and spin-dried to obtain the compound (UB-019b, 200 mg, 85.1% yield) as a light yellow solid. The crude product was directly used in the next step. LCMS [M+1]=556.6

### Step 2: UB-019

Compound UB-019b (200 mg, 0.45 mmol) was dissolved in methanol (3 mL), 1N hydrochloric acid methanol gas (3 mL) was added under stirring, and the reaction was carried out at 25° C. for 14 hours. The reaction solution was spin-dried, the pH of which was adjusted to 9 with saturated sodium carbonate solution, and extracted with ethyl acetate (20 mL*3). The organic layer was washed with brine, dried over anhydrous sodium sulfate, and spin-dried. The residue was separated and purified by column chromatography (dichloromethane/methanol=13/1) to obtain the compound (UB-019, 26 mg, 11.4% yield) as a light yellow solid. LCMS [M+1]⁺=556.6

### Example 20: Synthesis of Compound UB-020

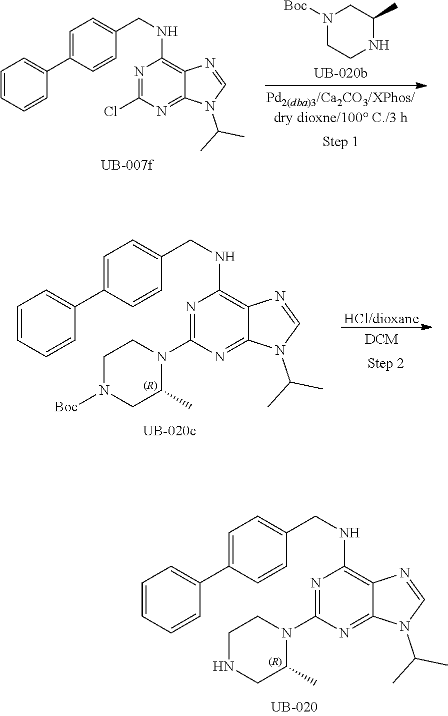

### Step 1: UB-020c

Tert-butyl (R)-4-(6-(([1,1′-biphenyl]-4-ylmethyl)amino)-9-isopropyl-9H-purin-2-yl)-3-methylpiperazin-1-carboxylate

To a solution of UB-007f (200 mg, 0.530 mmol) and UB-020b (424 mg, 0.120 mmol) in anhydrous dioxane (10 mL) were added Pd₂(dba)₃ (48 mg, 0.054 mmol), Cs2CO₃ (518 mg, 1.59 mmol) and XPhos (126 mg, 0.265 mmol) under Ar. The mixture was stirred at 100° C. for 3 hours. The reaction solution was concentrated and purified by flash chromatography using PE/EA=0-50% as eluent to obtain a white solid compound (UB-020c, 110 mg, 38.3% yield). LCMS [M+1]⁺=542.4

### Step 2: UB-020

### (R)—N-([1,1′-biphenyl]-4-ylmethyl)-9-isopropyl-2-(2-methylpiperazin-1-yl)-9H-purin-6-amine

UB-020c (110 mg, 0.203 mmol) and 8 mL of DCM solution were added to a reaction flask, and a solution of HCl in dioxane (3 mL) was added. The mixture was stirred at room temperature for 1 hour. The reaction mixture was quenched with NaHCO₃ (5 mL) and extracted with DCM (10 mL*3). The organic phase was separated, washed with brine (10 mL*3) and dried over Na₂SO₄, then concentrated and purified by flash chromatography, using DCM: 10% MeOH/DCM=0-100% as eluent, to obtain a white solid compound (UB-020, 42.5 mg, yield 47.5%) LCMS [M+1]⁺=442.3 ¹H NMR (400 MHz, DMSO-d₆) δ 7.99 (s, 1H), 7.84 (s, 1H), 7.64-7.59 (m, 2H), 7.58 (d, J=8.2 Hz, 2H), 7.47-7.40 (m, 4H), 7.33 (t, J=7.3 Hz, 1H), 4.70 (dd, J=15.0, 11.6 Hz, 2H), 4.56 (dt, J=13.4, 6.7 Hz, 2H), 4.38-4.29 (m, 1H), 3.00-2.83 (m, 2H), 2.79 (s, 2H), 1.47 (dd, J=6.7, 1.0 Hz, 6H), 1.08 (d, J=6.5 Hz, 3H).

### Example 21: Synthesis of Compound UB-021

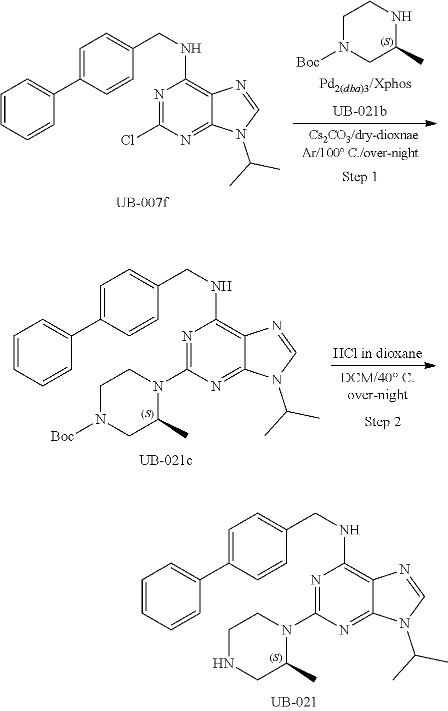

### Step 1: UB-021c

### Tert-butyl (S)-4-(6-(([1,1′-biphenyl]-4-ylmethyl)amino)-9-isopropyl-9H-purin-2-yl)-3-methylpiperazin-1-carboxylate

Compound UB-021b (637 mg, 3.18 mmol), compound UB-007f (200 mg, 0.53 mmol), cesium carbonate (519 mg, 1.59 mmol), Pd₂(dba)₃ (73 mg, 0.08 mmol) and Xphos (142 mg, 0.30 mmol) were dissolved in (6 mL) ultra-dry dioxane and the resulting mixture was reacted at 100° C. overnight. After the reaction was completed, the reaction solution was cooled to room temperature, concentrated and purified by silica gel column (EA/PE=40%) to obtain the compound (UB-021c, 280 mg, 97% yield) as a yellow solid. LCMS [M+1]⁺=542.3.

### Step 2: UB-021

### (S)—N-([1,1′-biphenyl]-4-ylmethyl)-9-isopropyl-2-(2-methylpiperazin-1-yl)-9H-purin-6-amine

Compound UB-021c (280 mg, 0.52 mmol) was dissolved in dichloromethane (6 mL), and hydrochloric acid in dioxane (6 mmol, 1.5 mL) was added. The reaction was stirred at 40° C. for 4 hours. After the reaction was completed, the reaction solution was added with saturated sodium bicarbonate solution to adjust the pH to 8-9, and extracted with DCM (40 mL*3). The organic phases were combined, washed with saturated salt water, and dried over anhydrous sodium sulfate. The extract was concentrated and purified by silica gel column (DCM/MeOH=30%) and lyophilized to give the desired compound UB-021 (40 mg, 24% yield) as a white solid. LCMS [M+1]⁺=442.3. ¹H NMR (400 MHz, DMSO-d₆) δ 8.11 (s, 1H), 7.88 (s, 1H), 7.60 (dd, J=13.8, 8.0 Hz, 4H), 7.47-7.42 (m, 4H), 7.34 (t, J=7.3 Hz, 1H), 4.87 (s, 1H), 4.56 (ddd, J=33.3, 26.3, 16.3 Hz, 4H), 3.15 (d, J=12.0 Hz, 1H), 3.09-2.90 (m, 3H), 2.74 (t, J=22.8 Hz, 1H), 1.47 (d, J=6.7 Hz, 6H), 1.24 (d, J=6.0 Hz, 1H), 1.12 (d, J=6.5 Hz, 3H).

### Example 22: Synthesis of Compound UB-022

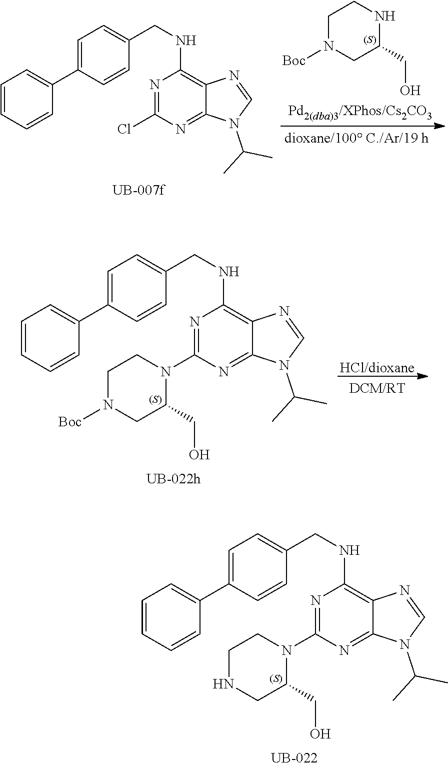

### Step 1: UB-022h

Compound UB-007f (270 mg, 0.72 mmol), UB-022g (416 mg, 2.34 mmol), Cs2CO₃ (624 mg, 2.15 mmol), Pd₂(dba)₃ (90 mg, 0.10 mmol) and XPhos (234 mg, 0.49 mmol) were added sequentially into a three-necked flask. The mixture was replaced with argon three times, then anhydrous 1,4-dioxane (16 mL) was added, and the mixture was stirred at 100° C. for 19 hours. The mixture was cooled to room temperature, concentrated under reduced pressure and purified by silica gel column chromatography (DCM: 10% MeOH in DCM=0-78%) to obtain the product UB-022h (65 mg, yield: 16%). LCMS [M+1]⁺=558.3

### Step 2: UB-022

To a mixture of compound UB-022h (65 mg, 0.12 mmol) in CH₂Cl₂ (3 mL) was added HCl/dioxane (4 N, 1.5 mL). The reaction mixture was stirred at 25° C. for 17 hours. The reaction mixture was concentrated and adjusted to pH=8-9 with saturated aqueous Na₂CO₃ solution. After extraction with CH₂Cl₂ (20 mL*3), the combined organic layer was washed with brine, and dried over anhydrous Na₂SO₄, and then the mixture was concentrated under reduced pressure and purified by reverse phase column chromatography to obtain product UB-022 (15.7 mg, yield: 29%) as a white solid. LCMS [M+1]⁺=458.3. 1H NMR (400 MHz, DMSO-d₆) δ 8.00 (s, 1H), 7.84 (s, 1H), 7.65-7.60 (m, 2H), 7.58 (d, J=8.2 Hz, 2H), 7.48-7.40 (m, 4H), 7.33 (t, J=7.3 Hz, 1H), 4.67-4.51 (m, 4H), 4.43 (d, J=12.9 Hz, 1H), 3.79 (t, J=9.3 Hz, 1H), 3.42 (dd, J=9.8, 4.7 Hz, 2H), 3.23 (d, J=12.3 Hz, 2H), 3.00-2.87 (m, 2H), 2.68 (dd, J=12.3, 4.0 Hz, 1H), 2.64-2.54 (m, 1H), 1.46 (d, J=6.2 Hz, 6H).

### Example 23: Synthesis of Compound UB-023

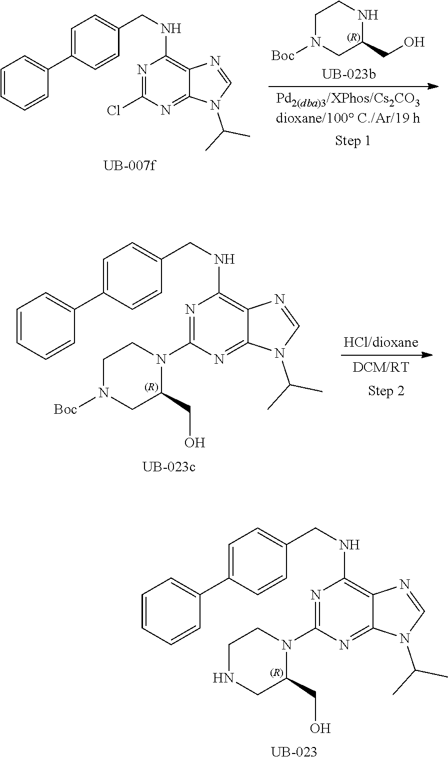

### Step 1: UB-023c

### Tert-butyl (R)-4-(6-(([1,1′-biphenyl]-4-ylmethyl)amino)-9-isopropyl-9H-purin-2-yl)-3-(hydroxymethyl)piperazin-1-carboxylate

To a reaction flask was added UB-007f (300 mg, 0.8 mmol), UB-023b (520 mg, 2.4 mmol) and anhydrous dioxane solution (18 mL), Pd₂(dba)₃ (80 mg, 0.08 mmol), Cs2CO₃ (800 mg, 2.4 mmol) and XPhos (200 mg, 0.4 mmol) under Ar. The mixture was stirred at 100° C. for 3 hours. The reaction was concentrated and purified by flash chromatography using DCM/(DCM:MeOH=10:1)═0-40% as eluent to obtain a yellow oil compound (UB-023c, 200 mg, yield 44.8%). LCMS [M+1]⁺=558.3

### Step 2: UB-023

### (R)-(1-(6-(([1,1′-biphenyl]-4-ylmethyl)amino)-9-isopropyl-9H-purin-2-yl)piperazin-2-yl)methanol

UB-023c (200 mg, 0.359 mmol), 8 mL DCM and HCl in dioxane (2 mL) were added to a reaction flask. The mixture was stirred at room temperature for 1 hour. The reaction mixture was quenched with NaHCO₃ (4 mL) and extracted with DCM (20 mL*3). The organic phase was separated, washed with brine (20 mL*3) and dried over Na₂SO₄, then concentrated and purified by flash chromatography, using DCM/(DCM:MeOH=10:1)═0-100% as eluent, to obtain a light yellow solid compound (UB-023, 25.5 mg, yield 15.5%) LCMS [M+1]⁺=458.3. ¹H NMR (400 MHz, DMSO-d₆) δ 8.09 (s, 1H), 7.87 (s, 1H), 7.65-7.60 (m, 2H), 7.58 (d, J=8.2 Hz, 2H), 7.48-7.41 (m, 4H), 7.34 (t, J=7.3 Hz, 1H), 4.70 (s, 1H), 4.67-4.58 (m, 2H), 4.54 (dd, J=13.3, 5.7 Hz, 2H), 3.78 (t, J=9.2 Hz, 1H), 3.49 (dd, J=10.1, 4.8 Hz, 1H), 3.37 (s, 1H), 3.07 (dd, J=24.9, 12.1 Hz, 2H), 2.84 (dd, J=12.3, 3.7 Hz, 1H), 2.71 (dd, J=23.4, 11.4 Hz, 1H), 1.50-1.44 (m, 6H).

### Example 24: Synthesis of Compound UB-024

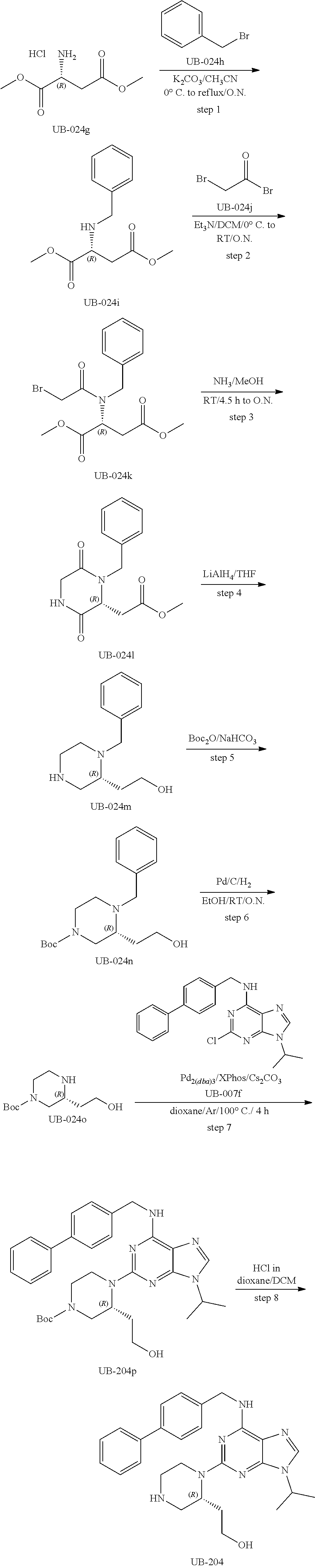

### Step 1: UB-024i

To a stirred suspension of K₂CO₃ (8.39 g, 60.17 mmol) in MeCN (100 mL) was added compound UB-024g (4.00 g, 20.24 mmol) at room temperature. The resulting mixture was stirred for 15 minutes, and then benzyl bromide (2.6 mL, 21.89 mmol) was added. The reaction mixture was stirred under reflux for 14 hours. The reaction mixture was concentrated and purified by silica gel chromatography (40 g, 45 mL/min; DCM: 10% MeOH in DCM=0-6-30%) to obtain UB-024i (3.95 g, 77% yield) as a white solid. LCMS [M+1]⁺=252.1

### Step 2: UB-024k

To a stirred cooled solution of compound UB-024i (3.95 g, 15.73 mmol) in DCM (80 mL) was added Et₃N (3.3 mL, 23.74 mmol) followed by the slow addition of bromoacetyl bromide (3.81 g, 18.88 mmol). The reaction mixture was warmed to room temperature and stirred overnight. The mixture was concentrated under reduced pressure and purified by silica gel chromatography (PE:EA=0-15-30%) to obtain UB-024k (4.62 g, 79% yield) as a colorless liquid. LCMS [M+1]⁺=372.1 & 374.1

### Step 3: UB-0241

Compound UB-024k (4.62 g, 12.43 mmol) was dissolved in CH₃OH (20 mL), and then a solution of NH₃ in CH₃OH (7N, 1.5 mL) was added, and the mixture was stirred at room temperature for 17 hours. Then the solvent was removed under reduced pressure and the residue was purified by silica gel column chromatography (25 g, 45 mL/min; CH₂C₂/10% CH₃OH in CH₂C₂=0-30%) to obtain product UB-0241 (2.18 g, yield: 64%) as a white solid. LCMS [M+1]⁺=277.1

### Step 4: UB-024m

To a stirred solution of UB-0241 (2.18 g, 7.92 mmol) in anhydrous tetrahydrofuran (30 mL) that had been cooled to 0° C. in an ice bath, was slowly added a solution of LiAlH₄ in THF (48 mL, 48 mmol). The resulting suspension was kept at 0° C. for 10 minutes and then refluxed for 1.5 hours. The reaction was then cooled down and water and sodium sulfate were added directly to the mixture until gas release was complete. The mixture was then filtered through diatomite, the filter cake was washed with MeOH, and the solvent was removed to give the product UB-024m (1.70 g, 75% yield) as a light orange oil. LCMS [M+1]⁺=221.2

### Step 5: UB-024n

To a solution of compound UB-024m (1.74 g, 7.90 mmol) in (40 mL) was added a solution of sodium bicarbonate (1.99 g, 23.69 mmol) in water (20 mL) at room temperature. To the above mixture was slowly added di-tert-butyl dicarbonate (2.59 g, 11.87 mmol), and the mixture was stirred at room temperature for 17 hours. The mixture was concentrated under reduced pressure and purified by silica gel chromatography (25 g, 45 mL/min; DCM: 10% MeOH in DCM=0-10%) to obtain product UB-024n (1.78 g, 71% yield) as a light yellow liquid. LCMS [M+1]⁺=321.2

### Step 6: UB-024o

Pd/C was added to a solution of compound UB-024n (1.78 g, 5.56 mmol) in EtOH (40 mL) at room temperature. The mixture was degassed and refilled with H2 three times. Then the mixture was stirred at room temperature for 24 hours. The mixture was filtered through a diatomite pad and washed with MeOH, and the filtrate was concentrated to give product UB-024o (1.28 g, yield 100%). LCMS [M+1]⁺=231.2

### Step 7: UB-024p

Compound UB-007f (170 mg, 0.45 mmol), UB-024o (225 mg, 0.98 mmol), Cs2CO₃ (440 mg, 1.35 mmol), Pd₂(dba)₃ (43 mg, 0.05 mmol) and XPhos (112 mg, 0.23 mmol) were added into a three-necked round bottom flask. The mixture was degassed and refilled with argon 3 times. Dry dioxane (14 mL) was added to the mixture via syringe. Then the mixture was stirred at 100° C. for 4 hours. The reaction mixture was concentrated and purified by silica gel chromatography (DCM: 10% MeOH in DCM=0-24%) to obtain UB-024p (158 mg, 61% yield) as a yellow solid. LCMS [M+1]⁺=572.3

### Step 8: UB-024

To a mixture of compound UB-024p (158 mg, 0.28 mmol) in CH₂Cl₂ (6 mL) was added HCl/dioxane (4 N, 1.5 mL). The reaction mixture was stirred at 25° C. for 17 hours. The reaction mixture was concentrated and adjusted to pH=8-9 with aqueous Na₂CO₃ solution. Then the mixture was concentrated under reduced pressure and purified by silica gel chromatography (DCM: 10% MeOH in DCM=0-33%) to obtain UB-024 (41 mg, 32% yield) as a white solid. LCMS [M+1]⁺=472.3 ¹H NMR (400 MHz, DMSO-d₆) δ 7.97 (s, 1H), 7.83 (s, 1H), 7.62 (dd, J=5.2, 3.3 Hz, 2H), 7.58 (d, J=8.3 Hz, 2H), 7.44 (dd, J=8.0, 6.8 Hz, 4H), 7.37-7.29 (m, 1H), 4.65 (s, 3H), 4.53 (m, 1H), 4.44 (d, J=11.2 Hz, 1H), 3.36 (t, J=6.9 Hz, 2H), 2.91-2.77 (m, 3H), 2.69-2.60 (m, 1H), 1.84 (d, J=6.6 Hz, 2H), 1.46 (dd, J=6.7, 3.4 Hz, 6H).

### Example 25: Synthesis of Compound UB-025

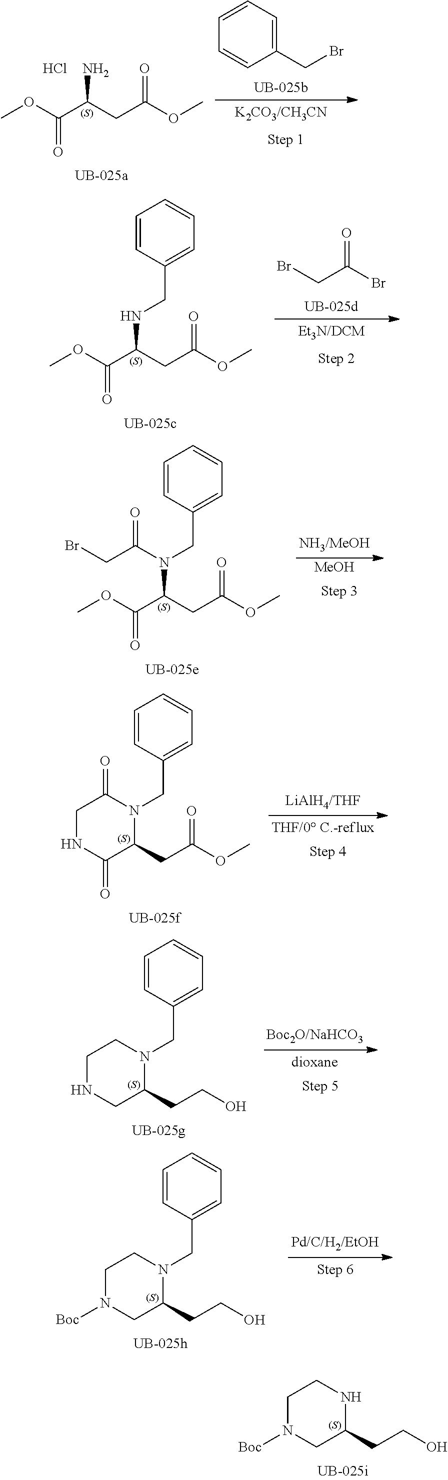

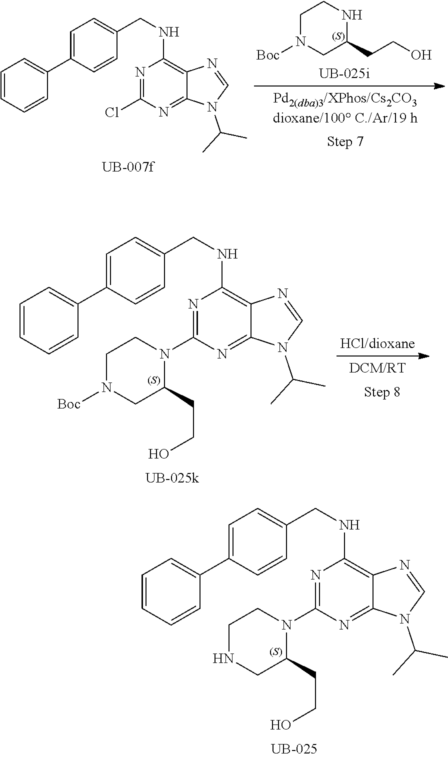

### Step 1: UB-025c

### Dimethylbenzyl-L-aspartic acid

K₂CO₃ (6.29 g, 45.54 mmol) and 20 mL of MeCN were added to a reaction flask at room temperature. UB-025a (3.0 g, 15.18 mmol) was added to the stirred suspension. The reaction solution was stirred for 15 minutes, added with UB-025b (2.726 g, 15.94 mmol). The reaction mixture was stirred under reflux for 16 hours. The solvent was evaporated and the residue was extracted with DCM (3×50 mL) and dried over Na₂SO₄. The solvent was evaporated under reduced pressure. The residue was purified by column chromatography with DCM/(DCM:MeOH=10:1)═0-10% to obtain a colorless oil compound (UB-025c, 3.0 g, yield 78.7%). LCMS [M+1]⁺=252.1

### Step 2: UB-025e

### Dimethyl N-Benzyl-N-(2-bromoacetyl)-L-aspartate

UB-025c (3.0 g, 12 mmol), anhydrous DCM (15 mL) and Et₃N (1.82 g, 18 mmol) were added into a reaction flask. The mixture was cooled to 0° C. and a solution of UB-025d (1.89 g, 14.3 mmol) in 5 mL of anhydrous DCM was slowly added. The reaction was stirred at room temperature for 1 hour. The brown solution was washed twice with 1 M HCl solution, twice with saturated NaHCO₃ solution and once with brine. The organic phase was dried over Na₂SO₄, then concentrated and purified by flash chromatography using PE/EA=0-20% to obtain a light yellow oil compound (UB-025e, 3.1 g, yield 69.6%). LCMS [M+1]⁺=372.0; 374.0

### Step 3: UB-025f

### Methyl S)-2-(1-benzyl-3,6-dioxopiperazin-2-yl)acetate

A solution of UB-025e (3.1 g, 8.36 mmol) and CH₃OH (10 mL) was added to a reaction flask. The mixture was stirred evenly, and then added with 6 mL of 7M NH₃/CH₃OH solution, and the mixture was stirred at room temperature for 7 hours. The solvent was then removed and the residue was purified by flash chromatography (DCM/MeOH=0-30%) to obtain a white solid compound (UB-025f, 1.6 g, yield 69.3%). LCMS [M+1]⁺=277.1

### Step 4: UB-025g

### (S)-2-(1-Benzylpiperazin-2-yl)ethan-1-ol

UB-025f (500 mg, 1.81 mmol) and 10 mL of anhydrous THF solution were added to a reaction flask under an ice bath, and then LiAlH₄/THF (10.9 mL, 1 mmol/mL) solution was slowly added dropwise. The resulting suspension was stirred at 0° C. for 10 minutes and then refluxed for 90 minutes. The reaction mixture was quenched with Na₂SO₄ and H₂O. The mixture was filtered through diatomite, then concentrated to obtain a yellow oil compound (UB-025g, 400 mg, yield 100%), which was directly used in the next reaction. LCMS [M+1]⁺=221.2

### Step 5: UB-025h

### Tert-butyl (S)-4-Benzyl-3-(2-hydroxyethyl)piperazin-1-carboxylate

NaHCO₃ (1570 mg, 18.7 mmol) and water (10 mL) were added to a solution of UB-025g (380 mg, 1.7 mmol) in 1,4-dioxane (10 mL) at room temperature. (Boc)₂O (524 mg, 2.4 mmol) was added and the mixture was stirred at room temperature for 1 hour. After completion of the reaction, water (20 mL) and EA (20 mL) were added and the layers were separated. The aqueous layer was extracted again with EA (20 mL). The combined organic phases were washed with water (30 mL) and saturated aqueous sodium chloride solution (20 mL), dried over sodium sulfate, filtered, and concentrated in vacuo. The resulting oil was purified by silica gel flash chromatography using DCM/(DCM:MeOH=10:1)═10-30% as eluent to obtain a yellow oil compound (UB-025h, 500 mg, yield 91.9%). LCMS [M+1]⁺=321.3

### Step 6: UB-025i

### Tert-butyl (S)-3-(2-hydroxyethyl)piperazin-1-carboxylate

Pd/C (50 mg, 10% wt) was added to a vessel purged with nitrogen. EtOH (5 mL) was added and then a solution of UB-025h (500 mg) in EtOH (5 mL) was added. The vessel was sealed, purged with nitrogen, purged with hydrogen, and reacted at room temperature under hydrogen pressure overnight. The reaction mixture was filtered and concentrated in vacuo to afford a yellow oil compound (1246i, 200 mg, yield 58.2%). LCMS [M+1]⁺=231.2

### Step 7: UB-025k

### Tert-butyl (S)-4-(6-(([1,1′-biphenyl]-4-ylmethyl)amino)-9-isopropyl-9H-purin-2-yl)-3-(2-hydroxyethyl)piperazin-1-carboxylate

Under Ar, to a reaction flask was added UB-025i (156 mg, 0.413 mmol), UB-007f (190 mg, 0.825 mmol), Pd₂(dba)₃ (38 mg, 0.041 mmol), Cs2CO₃ (404 mg, 1.24 mmol), XPhos (100 mg, 0.21 mmol) and anhydrous dioxane (10 mL). The mixture was stirred at 100° C. for 3 hours. The reaction was concentrated and purified by flash chromatography using DCM/(DCM:MeOH=10:1)═0-40% as eluent to obtain a yellow solid compound (UB-025k, 90 mg, yield 38%). LCMS [M+1]⁺=572.3

### Step 8: UB-025 047

### (S)-2-(1-(6-(([1,1′-biphenyl]-4-ylmethyl)amino)-9-isopropyl-9H-purin-2-yl)piperazin-2-yl)ethan-1-ol

A solution of UB-025k (90 mg, 0.16 mmol) and 6 mL of DCM was added to a reaction flask, the mixture was stirred evenly, and TFA (1 mL) was added. The mixture was stirred at room temperature for 1 hour. The reaction mixture was quenched with NaHCO₃ and extracted with DCM (10 mL*3). The organic phase was separated, washed with brine (10 mL*3) and dried over Na₂SO₄, then concentrated and purified by flash chromatography, using DCM/(DCM:MeOH=10:1)═0-100% as eluent, to obtain a white solid compound (UB-025, 25 mg, yield 33.8%) LCMS [M+1]⁺=472.2. ¹H NMR (400 MHz, DMSO-d₆) δ 8.00 (s, 1H), 7.84 (s, 1H), 7.64-7.60 (m, 2H), 7.58 (d, J=8.2 Hz, 2H), 7.47-7.41 (m, 4H), 7.34 (d, J=7.4 Hz, 1H), 4.66 (d, J=30.1 Hz, 3H), 4.54 (dd, J=13.5, 6.8 Hz, 1H), 4.50-4.44 (m, 1H), 3.37 (t, J=6.7 Hz, 3H), 2.91 (t, J=10.9 Hz, 3H), 2.75-2.64 (m, 1H), 1.93-1.80 (m, 2H), 1.46 (dd, J=6.7, 3.2 Hz, 6H).

### Example 26: Synthesis of Compound UB-026

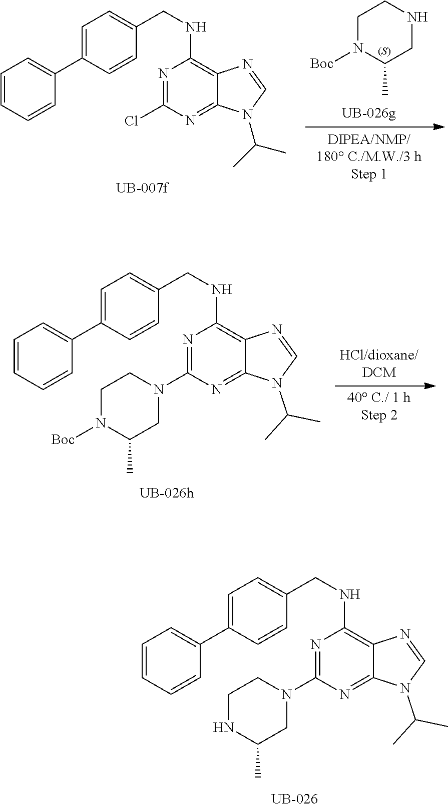

### Step 1: UB-026h

Compound UB-007f (80 mg, 0.11 mmol) and UB-026g (210 mg, 1.05 mmol) were dissolved in N-methylpyrrolidone (2 mL), N,N-diisopropylethylamine (216 mg, 1.68 mmol) was added, and then the mixture was reacted at 180° C. under microwave for 3 hours. The reaction mixture was cooled to room temperature and added with water (10 mL), and solids were precipitated. The precipitate was filtered and washed with water (20 mL). The solid was dissolved with dichloromethane and then concentrated. The residue was purified by silica gel and gel column chromatography (petroleum ether/ethyl acetate=2/1) to obtain the desired product (UB-026h, 84 mg, yield 74%) as a yellow liquid. LCMS[M+1]⁺=542.

### Step 2: UB-026

Compound UB-026h (84 mg, 0.15 mmol) was dissolved in dichloromethane (4 mL) and hydrochloric acid/dioxane in dioxane (1 mL), and then the mixture was stirred at 40° C. for 1 hour. The reaction mixture was concentrated and adjusted to pH=8-9 with saturated sodium bicarbonate aqueous solution. After extraction with dichloromethane (20 mL*3), the combined organic layers were washed with brine, dried over anhydrous sodium sulfate, filtered, concentrated, and the residual aqueous solution was lyophilized to obtain the desired product (UB-026, 20.2 mg, yield 30%) as a white solid. LCMS [M+1]⁺=442. ¹H NMR (400 MHz, CDCl₃) δ 7.99 (s, 1H), 7.84 (s, 1H), 7.65-7.54 (m, 4H), 7.44 (dt, J=7.8, 3.6 Hz, 4H), 7.38-7.29 (m, 1H), 4.57 (td, J=13.5, 13.0, 6.2 Hz, 3H), 4.44 (dd, J=12.7, 3.0 Hz, 2H), 2.86 (d, J=11.1 Hz, 1H), 2.68 (td, J=12.0, 2.7 Hz, 1H), 2.57 (td, J=11.4, 4.7 Hz, 3H), 2.31 (dd, J=12.5, 10.3 Hz, 1H), 1.46 (d, J=6.7 Hz, 6H), 0.98 (d, J=6.2 Hz, 3H).

### Example 27: Synthesis of Compound UB-027

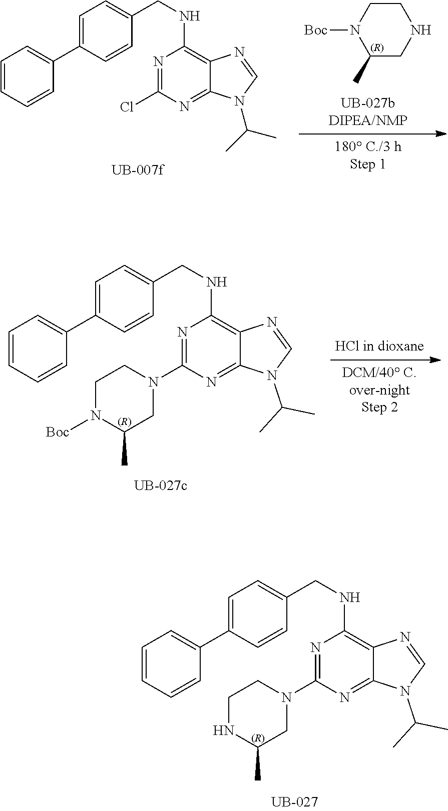

### Step 1: UB-027c

### Tert-butyl (R)-4-(6-(([1′-biphenyl]-4-ylmethyl)amino)-9-isopropyl-9H-purin-2-yl)-2-methylpiperazin-1-carboxylate

Compound UB-027b (2650 mg, 13.26 mmol) and compound UB-007f (1000 mg, 2.65 mmol) were dissolved in (7 mL) NMP and the mixture was reacted under microwave at 180° C. for 3 hours. The reaction solution was cooled to room temperature, water was added until solid precipitated from the solution, and the mixture was filtered. The residue was dissolved and purified by silica gel column (DCM/MeOH=10%) to obtain the compound (UB-027c, 712 mg, 50% yield and 470 mg of product (off-Boc)) as a yellow oil. LCMS [M+1]⁺=542.3.

### Step 2: UB-027

### (R)—N-([1,1′-biphenyl]-4-ylmethyl)-9-isopropyl-2-(3-methylpiperazin-1-yl)-9H-purin-6-amine

Compound UB-027c (712 mg, 1.32 mmol) was dissolved in dichloromethane (16 mL), and hydrochloric acid in 1,4-dioxane (16 mmol, 4 mL) was added. The reaction was stirred at 40° C. for 4 hours. After the reaction was completed, the reaction solution was added with saturated sodium bicarbonate solution to adjust the pH to 8-9, and extracted with DCM (40 mL*3). The organic phases were combined, washed with saturated salt water, and dried over anhydrous sodium sulfate. The extract was concentrated and lyophilized to give the desired compound UB-027 (525.9 mg, 45% yield) as a white solid. LCMS [M+1]⁺=442.3 ¹H NMR (400 MHz, DMSO-d₆) δ 7.98 (s, 1H), 7.84 (s, 1H), 7.64-7.55 (m, 4H), 7.47-7.41 (m, 4H), 7.33 (t, J=7.3 Hz, 1H), 4.56 (dt, J=13.4, 6.7 Hz, 3H), 4.44 (d, J=12.3 Hz, 2H), 2.85 (d, J=11.3 Hz, 1H), 2.67 (t, J=12.0 Hz, 1H), 2.61-2.53 (m, 2H), 2.30 (dd, J=12.3, 10.4 Hz, 1H), 1.46 (d, J=6.7 Hz, 6H), 0.97 (d, J=6.2 Hz, 3H).

### Example 28: Synthesis of Compound UB-028

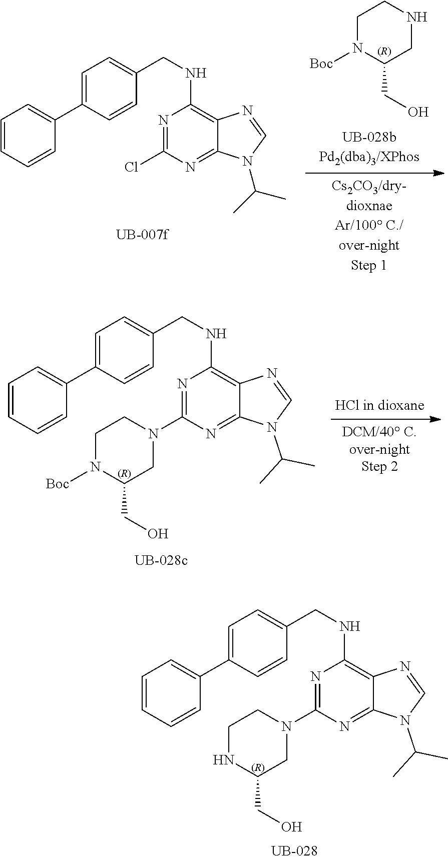

### Step 1: UB-028c

### (R)-Tert-butyl 4-(6-(([1,1′-biphenyl]-4-ylmethyl)amino)-9-isopropyl-9H-purin-2-yl)-2-(hydroxymethyl)piperazin-1-carboxylate

Compound UB-028b (287 mg, 1.33 mmol) and compound UB-007f (100 mg, 0.27 mmol) were dissolved in (3 mL) NMP and the mixture was reacted under microwave at 180° C. for 3 hours. The reaction solution was cooled to room temperature, water was added until solid precipitated from the solution, and the mixture was filtered. The filter residue was dissolved and purified by silica gel column (DCM/MeOH=30%) to obtain the compound (UB-028c, 42 mg, 59% yield) as a yellow oil. LCMS [M+1]⁺=558.3.

### Step 2: UB-028

### (R)-(4-(6-(([1,1′-biphenyl]-4-ylmethyl)amino)-9-isopropyl-9H-purin-2-yl)piperazin-2-yl)methanol

Compound UB-028c (42 mg, 0.07 mmol) was dissolved in dichloromethane (2 mL), and hydrochloric acid in 1,4-dioxane (2 mmol, 0.5 mL) was added. The reaction was stirred at 40° C. for 4 hours. After the reaction was completed, the reaction solution was added with saturated sodium bicarbonate solution to adjust the pH to 8-9, and extracted with DCM (40 mL*3). The organic phases were combined, washed with saturated salt water, and dried over anhydrous sodium sulfate. The extract was concentrated and lyophilized to give the desired compound UB-028 (17.5 mg, 54% yield) as a white solid. LCMS [M+1]⁺=458.2. ¹H NMR (400 MHz, DMSO-d₆) δ 8.06 (s, 1H), 7.87 (s, 1H), 7.60 (dd, J=16.3, 7.7 Hz, 4H), 7.48-7.41 (m, 4H), 7.33 (t, J=7.3 Hz, 1H), 4.87 (s, 1H), 4.69-4.42 (m, 5H), 3.42 (d, J=3.4 Hz, 2H), 3.01 (d, J=11.4 Hz, 1H), 2.85 (t, J=11.2 Hz, 1H), 2.79-2.54 (m, 3H), 1.47 (d, J=6.7 Hz, 6H).

### Example 29: Synthesis of Compound UB-029

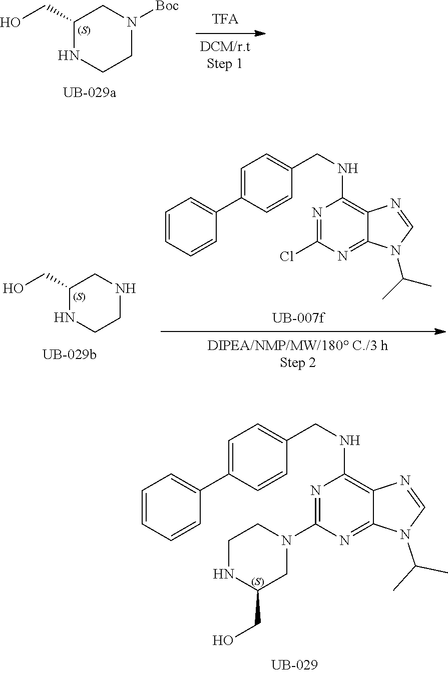

### Step 1: UB-029b

### (S)-Piperazin-2-ylmethanol

A solution of UB-029a (250 mg, 1.16 mmol) and 8 mL of DCM was added to a reaction flask, the mixture was stirred evenly, and TFA (2 mL) was added. The mixture was stirred at room temperature for 1 hour. The reaction mixture was concentrated to obtain a white solid compound (UB-029b, 400 mg, yield 100%) which was directly used in the next step. LCMS [M+1]⁺=117.1

### Step 2: UB-029

### (S)-(4-(6-(([1,1′-biphenyl]-4-ylmethyl)amino)-9-isopropyl-9H-purin-2-yl)piperazin-2-yl)methanol

A solution of UB-029b (153 mg, 1.323 mmol), UB-007f (100 mg, 0.265 mmol), DIPEA (274 mg, 2.12 mmol) and NMP (4 mL) was added to a microwave tube. The mixture was stirred at 180° C. under microwave condition for 3 hours. The reaction mixture was poured into H₂O. The resulting precipitate was collected by filtration, the filtrate was extracted with EA three times, and the organic phases were combined, filtered and purified by silica gel column chromatography [eluent: DCM/(10% MeOH/DCM)═0-100%] to obtain a white solid compound (UB-029, 62 mg, yield 51%). LCMS [M+1]⁺=458.3. ¹H NMR (400 MHz, DMSO-d₆) δ 8.00 (s, 1H), 7.85 (s, 1H), 7.64-7.60 (m, 2H), 7.58 (d, J=8.2 Hz, 2H), 7.49-7.41 (m, 4H), 7.33 (t, J=7.3 Hz, 1H), 4.68 (t, J=5.1 Hz, 1H), 4.61 (d, J=6.3 Hz, 1H), 4.56 (dd, J=13.4, 6.8 Hz, 2H), 4.45 (d, J=12.6 Hz, 1H), 3.38-3.34 (m, 2H), 2.93 (d, J=11.5 Hz, 1H), 2.76 (t, J=12.0 Hz, 1H), 2.60 (t, J=9.8 Hz, 2H), 2.48-2.39 (m, 1H), 1.46 (d, J=6.7 Hz, 6H).

### Example 30: Synthesis of Compound UB-030

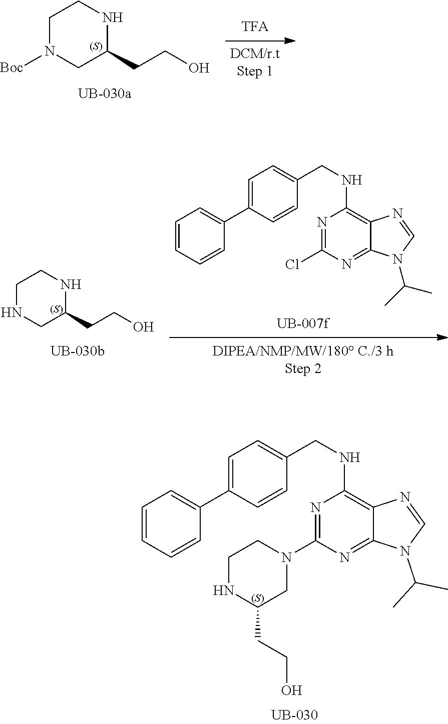

### Step 1: UB-030b

### (S)-2-(piperazin-2-yl)ethan-1-ol

A solution of UB-030a (460 mg, 2 mmol) and 8 mL of DCM was added to a reaction flask, the mixture was stirred evenly, and TFA (2 mL) was added. The mixture was stirred at room temperature for 1 hour. The reaction mixture was concentrated to obtain a white solid compound (UB-030b, 300 mg, yield 100%). The mixture was directly used in the next step. LCMS [M+1]⁺=131.2

### Step 2: UB-030

### (S)-2-(4-(6-(([1,1′-biphenyl]-4-ylmethyl)amino)-9-isopropyl-9H-purin-2-yl)piperazin-2-yl)ethan-1-ol

A solution of UB-030b (138 mg, 1.058 mmol), UB-007f (80 mg, 0.0.212 mmol), DIPEA (220 mg, 1.7 mmol) and NMP (4 mL) was added to a microwave tube. The mixture was stirred at 180° C. under microwave condition for 3 hours. The reaction mixture was poured into H₂O. The resulting precipitate was collected by filtration, the filtrate was extracted with EA three times, and the organic phases were combined, filtered and purified by silica gel column chromatography [eluent: DCM/(10% MeOH/DCM)═0-100%] to obtain a white solid compound (UB-030, 59 mg, yield 59.0%). LCMS [M+1]⁺=472.3. ¹H NMR (400 MHz, DMSO-d₆) δ 8.01 (s, 1H), 7.84 (s, 1H), 7.64-7.60 (m, 2H), 7.58 (d, J=8.2 Hz, 2H), 7.44 (t, J=8.1 Hz, 4H), 7.33 (t, J=7.3 Hz, 1H), 4.57 (td, J=13.3, 6.5 Hz, 3H), 4.48 (d, J=10.8 Hz, 1H), 4.42 (d, J=12.8 Hz, 1H), 3.53 (td, J=6.4, 2.2 Hz, 2H), 2.90 (d, J=11.8 Hz, 1H), 2.76 (d, J=11.5 Hz, 1H), 2.62 (dd, J=16.8, 10.5 Hz, 2H), 2.45 (d, J=10.3 Hz, 1H), 1.54-1.48 (m, 2H), 1.46 (d, J=6.8 Hz, 6H).

### Example 31: Synthesis of Compound UB-031

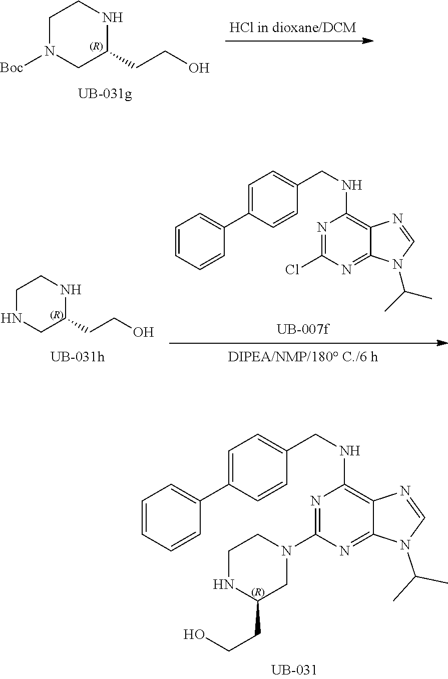

### Step 1: UB-031h

To a mixture of compound UB-031h (313 mg, 1.36 mmol) in CH₂Cl₂ (3 mL) was added HCl/dioxane (4 N, 3.5 mL). The reaction mixture was stirred at 25° C. for 17 hours. The reaction mixture was concentrated and adjusted to pH=8-9 with saturated aqueous Na₂CO₃ solution. After extraction with CH₂C₂ (20 mL*3), the combined organic layer was washed with brine, dried over anhydrous Na₂SO₄, and then the mixture was concentrated under reduced pressure to obtain the product UB-031h (177 mg, yield: 100%) as a white solid. LCMS [M+1]⁺=131.2 Step 2: UB-031

Compound UB-007f (70 mg, 0.19 mmol), compound UB-031h (177 mg, 0.47 mmol), DIPEA (192 mg, 1.48 mmol) and N-methylpyrrolidone (3 mL) were added to a microwave tube respectively, and the mixture was heated at 180° C. for 6 hours. The mixture was cooled to room temperature, concentrated under reduced pressure and purified by silica gel column chromatography (4 g, 40 mL/min; DCM: 10% MeOH in DCM=0-33%) to obtain crude product (48 mg, 55% yield) as a brownish yellow liquid. The crude product was further purified by preparative liquid chromatography (Prep. HPLC, mobile phase: HCl/water/acetonitrile) to obtain the product UB-031 (31.5 mg, yield: 36%) as a yellow solid. LCMS [M+1]⁺=472.3. ¹H NMR (400 MHz, DMSO-d₆) δ 8.19 (s, 1H), 7.91 (s, 1H), 7.64-7.56 (m, 5H), 7.47-7.41 (m, 5H), 7.33 (dd, J=10.4, 4.3 Hz, 2H), 4.65-4.45 (m, 6H), 3.56 (dd, J=11.3, 5.8 Hz, 2H), 3.18 (d, J=12.1 Hz, 1H), 3.09 (d, J=11.0 Hz, 3H), 2.87 (s, 3H), 1.67 (td, J=14.5, 7.0 Hz, 3H), 1.47 (d, J=6.8 Hz, 8H).

### Example 32: Synthesis of Compound UB-032

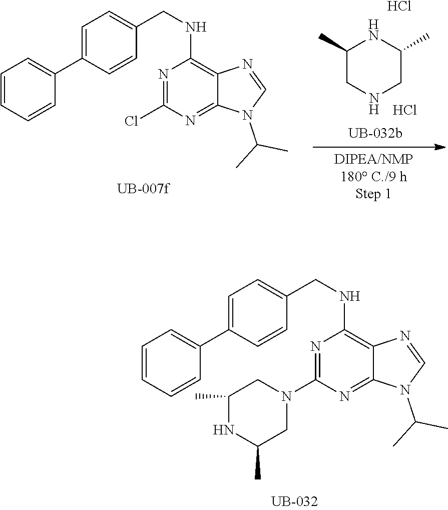

### Step 1: UB-032

### N-([1,1′-biphenyl]-4-ylmethyl)-9-cyclobutyl-2-(piperazin-1-yl)-9H-purin-6-amine

UB-007f (60 mg, 0.16 mmol), UB-032b (148 mg, 0.80 mmol), DIPEA (164 mg, 1.27 mmol), and NMP (3 mL) were added to a 10 mL microwave tube. The resulting mixture was reacted under microwave condition at 180° C. for 3 hours. After the reaction finished, 30 mL of water was added. The solid precipitated and was filtered. The filter cake was separated by column chromatography (eluent: DCM/(10% MeOH in DCM)═100/0-30/70) to obtain a white solid compound UB-032 (30 mg, yield 42%). LCMS [M+H]⁺=456.3. ¹H NMR (400 MHz, DMSO-d₆) δ 8.03 (s, 1H), 7.84 (s, 1H), 7.65-7.60 (m, 2H), 7.57 (d, J=8.2 Hz, 2H), 7.48-7.40 (m, 4H), 7.34 (d, J=7.3 Hz, 1H), 4.56 (dt, J=13.4, 6.7 Hz, 3H), 3.76 (dd, J=12.7, 3.0 Hz, 2H), 3.41 (dd, J=12.8, 6.2 Hz, 3H), 3.15 (s, 2H), 1.46 (dd, J=6.7, 4.7 Hz, 6H), 1.01 (d, J=6.3 Hz, 6H).

### Example 33: Synthesis of Compound UB-033

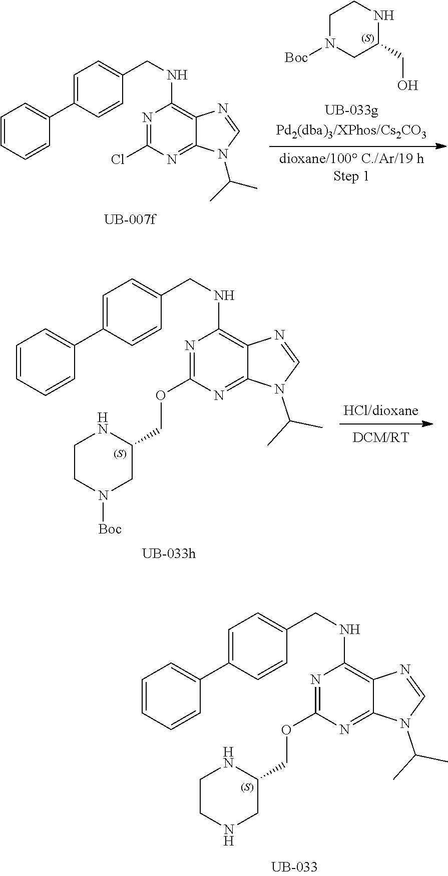

### Step 1: UB-033h

Compound UB-007f (270 mg, 0.72 mmol), UB-033g (416 mg, 2.34 mmol), Cs2CO₃ (624 mg, 2.15 mmol), Pd₂(dba)₃ (90 mg, 0.10 mmol) and XPhos (234 mg, 0.49 mmol) were added sequentially into a three-necked flask. The mixture was replaced with argon three times, then anhydrous 1,4-dioxane (16 mL) was added, and the mixture was stirred at 100° C. for 19 hours. The mixture was cooled to room temperature, concentrated under reduced pressure and purified by silica gel column chromatography (DCM: 10% MeOH in DCM=0-78%) to obtain the product UB-033h (109 mg, yield: 27%). LCMS [M+1]⁺=558.3

### Step 2: UB-033

To a mixture of compound UB-033h (109 mg, 0.20 mmol) in CH₂Cl₂ (3 mL) was added HCl/dioxane (4 N, 1.5 mL). The reaction mixture was stirred at 25° C. for 17 hours. The reaction mixture was concentrated and adjusted to pH=8-9 with saturated aqueous Na₂CO₃ solution. After extraction with CH₂Cl₂ (20 mL*3), the mixture was concentrated under reduced pressure and purified by silica gel column chromatography (DCM: 10% MeOH in DCM=0-100%) to obtain the product UB-033 (59.3 mg, 60% yield) as a light yellow solid. LCMS [M+1]⁺=458.3. ¹H NMR (400 MHz, DMSO-d₆) δ 7.94 (s, 1H), 7.82 (s, 1H), 7.65-7.60 (m, 2H), 7.58 (d, J=8.2 Hz, 2H), 7.48-7.41 (m, 4H), 7.33 (t, J=7.3 Hz, 1H), 4.62 (s, 2H), 4.55 (m, 3H), 4.37 (d, J=12.5 Hz, 1H), 3.80 (t, J=9.4 Hz, 1H), 3.42-3.35 (m, 2H), 3.16 (d, J=12.0 Hz, 1H), 2.86 (t, J=12.8 Hz, 2H), 2.58 (dd, J=12.1, 3.8 Hz, 1H), 1.46 (dd, J=6.7, 0.9 Hz, 6H).

### Example 34: Synthesis of Compound UB-034

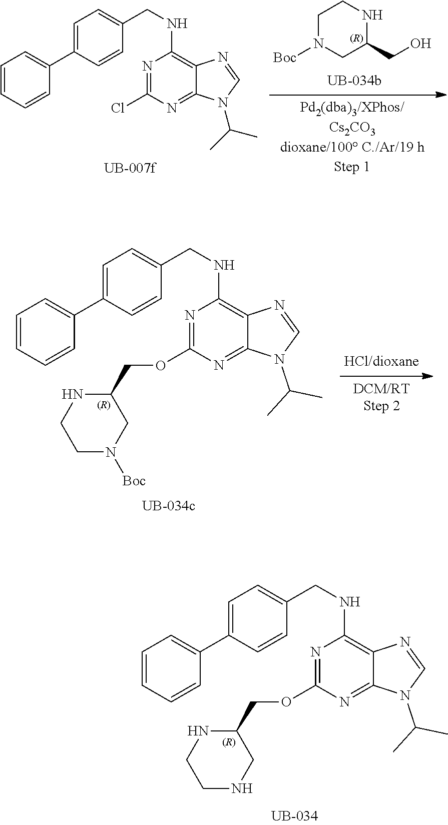

### Step 1: UB-034c

### Tert-butyl (R)-3-(((6-(([1,1′-biphenyl]-4-ylmethyl)amino)-9-isopropyl-9H-purin-2-yl) oxy)methyl)piperazin-1-carboxylate

Under Ar, to a reaction bottle was added a solution of UB-007f (300 mg, 0.8 mmol), UB-034b (520 mg, 2.4 mmol) and anhydrous dioxane (18 mL), and Pd₂(dba)₃ (80 mg, 0.08 mmol), Cs2CO₃ (800 mg, 2.4 mmol) and XPhos (200 mg, 0.4 mmol) were added. The mixture was stirred at 100° C. for 3 hours. The reaction was concentrated and purified by flash chromatography using DCM/(DCM:MeOH=10:1)═0-40% as eluent to obtain a yellow oil compound (UB-034c, 200 mg, yield 44.8%). LCMS [M+1]⁺=558.3

### Step 2: UB-034

### (R)—N-([1,1′-biphenyl]-4-ylmethyl)-9-isopropyl-2-(piperazin-2-ylmethoxy)-9H-purin-6-amine

A solution of UB-034c (200 mg, 0.359 mmol) and 8 mL of DCM and HCl in dioxane (2 mL) were added to a reaction flask. The mixture was stirred at room temperature for 1 hour. The reaction mixture was quenched with NaHCO₃ (4 mL) and extracted with DCM (20 mL*3). The organic phase was separated, washed with brine (20 mL*3) and dried over Na₂SO₄, then concentrated and purified by flash chromatography, using DCM/(DCM:MeOH=10:1)═0-100% as eluent, to obtain a light yellow solid compound (UB-034, 80.5 mg, yield 49%) LCMS [M+1]⁺=458.3. ¹H NMR (400 MHz, DMSO-d₆) δ 8.39 (s, 1H), 8.05 (s, 1H), 7.61 (dd, J=11.1, 7.8 Hz, 4H), 7.43 (dd, J=11.9, 5.2 Hz, 4H), 7.35 (d, J=7.3 Hz, 1H), 4.80-4.50 (m, 3H), 4.06 (d, J=5.6 Hz, 2H), 2.86 (d, J=9.2 Hz, 2H), 2.77 (d, J=10.6 Hz, 1H), 2.70 (d, J=11.1 Hz, 1H), 2.61-2.55 (m, 1H), 2.32 (d, J=10.7 Hz, 1H), 1.49 (d, J=6.7 Hz, 6H).

### Example 35: Synthesis of Compound UB-035

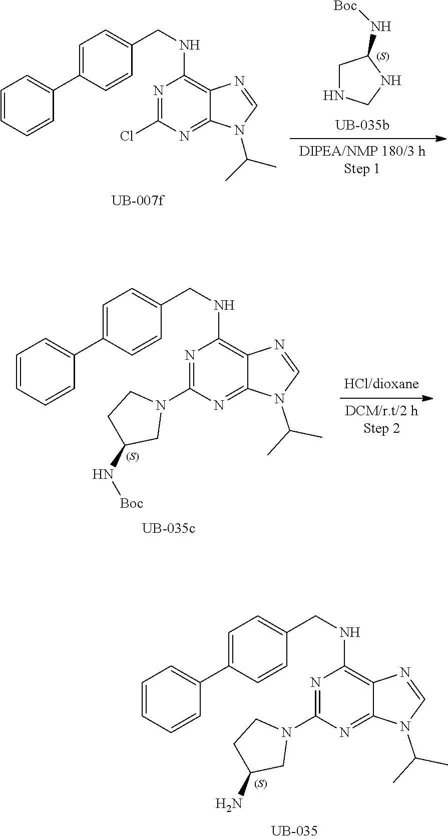

### Step 1: UB-035c

### Tert-butyl 4-(9-isopropyl-6-((propoxy-[1,1′-biphenyl]-4-yl)methyl)amino)-9H-purin-2-yl) piperazin-1-carboxylate

UB-007f (80 mg, 0.147 mmol), UB-035b (54 mg, 0.439 mmol), cesium carbonate (142 mg, 0.436 mmol), and DMF (5 mL) were added to a 100 ml round-bottom flask. The resulting mixture was reacted at room temperature for 1 hour. After the reaction finished, 20 mL of water was added, and the mixture was extracted three times with ethyl acetate. The organic solvent was dried over anhydrous sodium sulfate and spin-dried to obtain a yellow solid compound UB-035c (70 mg, yield 81%). LCMS [M+H]⁺=586.2

### Step 2: UB-035

### 9-isopropyl-2-(piperazin-1-yl)-N-((2-propoxy-[1,1′-biphenyl]-4-yl)methyl)-9H-purin-6-amine

UB-035c (70 mg, 0.11 mmol), dichloromethane (3 mL) and hydrochloric acid in dioxane (2 mL, 8 mmol) were added to a 50 mL round-bottom flask, and the resulting mixture was reacted at room temperature for 2 hours. The reaction was quenched with saturated sodium bicarbonate solution until the pH was neutral, and then extracted with dichloromethane. The organic phase was concentrated under reduced pressure and the residue was subjected to preparative chromatography to obtain a white solid compound UB-035 (14 mg, yield 25%). LCMS [M+1]⁺=428.3. ¹H NMR (400 MHz, DMSO-d₆) δ 7.93 (s, 1H), 7.80 (s, 1H), 7.66-7.55 (m, 4H), 7.51-7.40 (m, 4H), 7.33 (t, J=7.3 Hz, 1H), 4.65 (s, 1H), 4.56 (dt, J=13.4, 6.7 Hz, 1H), 3.66 (t, J=5.4 Hz, 1H), 3.59 (dd, J=11.5, 5.7 Hz, 2H), 3.53-3.44 (m, 2H), 3.27-3.18 (m, 2H), 2.06 (td, J=13.1, 6.8 Hz, 1H), 1.90 (s, 2H), 1.73 (dd, J=12.4, 6.4 Hz, 1H), 1.47 (d, J=6.8 Hz, 6H).

### Example 36: Synthesis of Compound UB-036

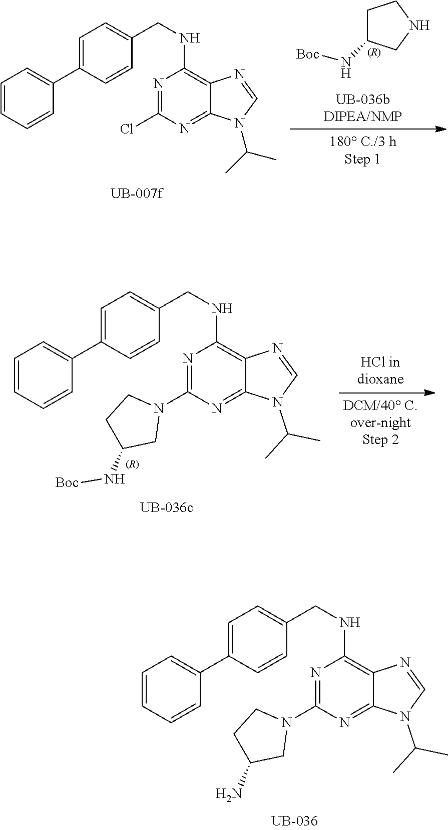

### Step 1: UB-036c

### Tert-butyl (R)-(1-(6-(([1,1′-biphenyl]-4-ylmethyl)amino)-9-isopropyl-9H-purin-2-yl) pyrrolidin-3-yl)carbamate

Compound UB-036b (198 mg, 1.06 mmol) and compound UB-007f (80 mg, 0.21 mmol) were dissolved in (2 mL) NMP and the mixture was reacted under microwave at 180° C. for 3 hours. The reaction solution was cooled to room temperature, water was added until solid precipitated from the solution, and the mixture was filtered. The filter residue was dissolved and purified by silica gel column (DCM/MeOH=10%) to obtain the compound (UB-036c, 25 mg, 57% yield) as a yellow oil. LCMS [M+1]⁺=528.3.

### Step 2: UB-036

### (R)—N-([1,1′-biphenyl]-4-ylmethyl)-2-(3-aminopyrrolidin-1-yl)-9-isopropyl-9H-purin-6-amine

Compound UB-036c (25 mg, 0.05 mmol) was dissolved in dichloromethane (2 mL), and hydrochloric acid in 1,4-dioxane (2 mmol, 0.5 mL) was added. The reaction was stirred at 40° C. for 4 hours. After the reaction was completed, the reaction solution was added with saturated sodium bicarbonate solution to adjust the pH to 8-9, and extracted with DCM (40 mL*3). The organic phases were combined, washed with saturated salt water, and dried over anhydrous sodium sulfate. The extract was concentrated and lyophilized to give the desired compound UB-036 (3.5 mg, 10% yield) as a white solid. LCMS [M+1]⁺=428.2. ¹H NMR (400 MHz, DMSO-d₆) δ 7.88 (s, 1H), 7.79 (d, J=5.9 Hz, 1H), 7.64-7.56 (m, 4H), 7.45 (dd, J=14.6, 7.5 Hz, 4H), 7.33 (t, J=7.3 Hz, 1H), 4.78-4.50 (m, 3H), 3.53 (dddd, J=17.9, 15.8, 14.0, 10.9 Hz, 4H), 3.13 (dd, J=10.8, 5.0 Hz, 1H), 1.99 (dt, J=12.3, 6.7 Hz, 1H), 1.63 (td, J=13.1, 6.6 Hz, 1H), 1.47 (d, J=6.8 Hz, 6H).

### Example 37: Synthesis of Compound UB-037

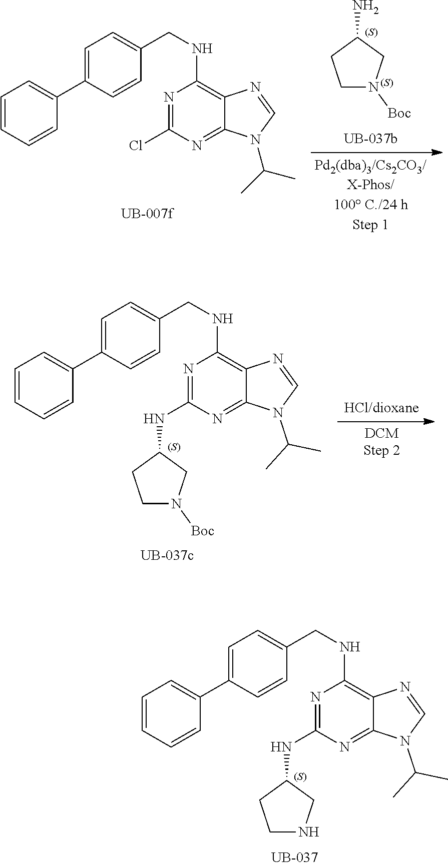

### Step 1: UB-037c

### Tert-Butyl (S)-3-((6-(([1,1′-biphenyl]-4-ylmethyl)amino)-9-isopropyl-9H-purin-2-yl)amino)pyrrolidin-1-carboxylate

UB-007f (100 mg, 0.265 mmol), UB-037b (160 mg, 0.8 mmol), Pd₂(dba)₃ (146.6 mg, 0.16 mmol), Cs2CO₃ (172.7 mg, 0.53 mmol), X-phos (380.8 mg, 0.8 mmol) and 1,4-dioxane (10 mL) were added to a reaction flask. The reaction mixture was replaced with argon three times and then heated to 100° C. and stirred overnight. The reaction mixture was diluted with water (30 mL), and then extracted with ethyl acetate (30 mL*3). The organic phase was separated, washed with brine (50 mL) and dried over anhydrous sodium sulfate, then concentrated and purified by chromatography (dichloromethane:dichloromethane/methanol 10/1=75:25) to obtain a yellow solid compound (UB-037c, 120 mg, crude, yield 35.58%). LCMS [M+1]⁺=528.2

### Step 2: UB-037

### N⁶—([1,1′-biphenyl]-4-ylmethyl)-9-isopropyl-N2-(pyrrolidin-3-yl)-9H-purin-2,6-diamine

To a 100 mL bottom flask were added UB-037c (121.2 mg, 0.23 mmol), a solution of hydrochloric acid in 1,4-dioxane (4 mL), and dichloromethane (8.0 mL) at room temperature. Then the solution was stirred at room temperature for 2 hours. The reaction mixture was filtered, and the filter cake was washed with dichloromethane (5 mL), neutralized with saturated aqueous sodium bicarbonate solution, and extracted with dichloromethane (3*50 mL). The combined organic phase was washed with brine, dried over anhydrous sodium sulfate and concentrated under reduced pressure, then added with methanol and water and freeze-dried to obtain a yellow solid product (UB-037, 54.7 mg, yield: 56.3%). LCMS [M+1]⁺=428.2. ¹H NMR (400 MHz, DMSO-d₆) δ 7.79 (s, 1H), 7.60 (dd, J=15.0, 7.9 Hz, 4H), 7.44 (t, J=7.6 Hz, 4H), 7.33 (t, J=7.3 Hz, 1H), 6.28 (d, J=6.6 Hz, 1H), 4.67 (s, 1H), 4.53 (dt, J=13.6, 6.7 Hz, 1H), 4.20 (d, J=6.2 Hz, 1H), 2.90 (dd, J=11.4, 6.6 Hz, 1H), 2.87-2.81 (m, 1H), 2.73-2.64 (m, 1H), 2.57 (dd, J=11.0, 4.2 Hz, 1H), 1.91 (dd, J=12.8, 6.3 Hz, 1H), 1.63-1.50 (m, 1H), 1.47 (d, J=6.7 Hz, 6H).

### Example 38: Synthesis of Compound UB-038

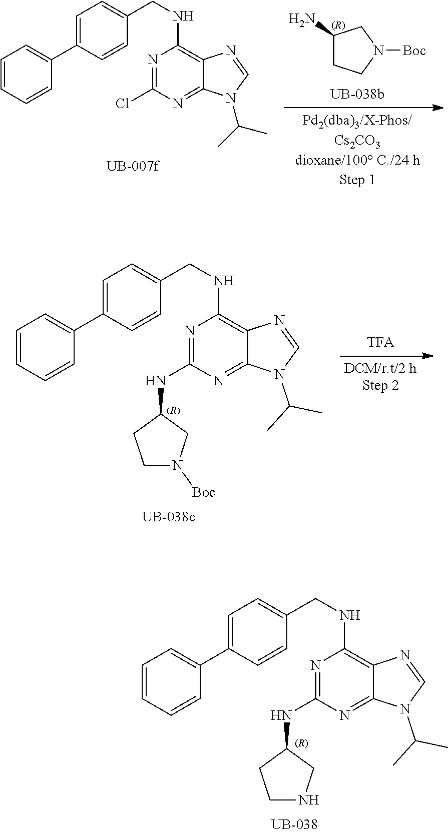

### Step 1: UB-038c

### (R)-Tert-butyl-3-(6-(([1,1′-biphenyl]-4-ylmethyl)amino)-9-isopropyl-9H-purin-2-yl)amino)pyrrolidin-1-carboxylate

To a 100 mL round-bottom flask were added in sequence compound UB-038a (400 mg, 1.06 mmol), UB-038b (1.18 g, 6.36 mmol), Pd₂(dba)₃ (97 mg, 0.106 mmol), XPhos (252 mg, 0.53 mmol), Cs2CO₃ (1.03 g, 3.18 mmol), and 1,4-dioxane (10 mL). The resulting mixture was replaced with argon three times and then heated to 100° C. and reacted for 24 h. After the reaction was completed, the mixture was concentrated under reduced pressure. The residue was separated by column chromatography (eluent: DCM:PE:EA=100/0-80/20) to obtain a light yellow solid compound UB-038c (200 mg, yield 36%). LCMS [M+H]⁺=528.3.

### Step 2: UB-038

### (R)—N⁶—([1,1′-biphenyl]-4-ylmethyl)-9-isopropyl-N2-(pyrrolidin-3-yl)-9H-purin-2,6-diamine

UB-038c (80 mg, 0.15 mmol), dichloromethane (3 mL) and trifluoroacetic acid (128 mg, 1.13 mmol) were added to a 50 mL round-bottom flask, and the resulting mixture was reacted at room temperature for 2 hours. The reaction was quenched with saturated sodium bicarbonate solution until the pH was neutral, and extracted with dichloromethane, and the organic phase was concentrated under reduced pressure. The residue was separated by column chromatography (eluent: DCM/(10% MeOH in DCM)═100/0-90/10) to obtain a white solid compound UB-038 (42 mg, yield 87%). LCMS [M+1]⁺=428.3. ¹H NMR (400 MHz, DMSO-d₆) δ 8.71 (s, 1H), 8.07 (s, 1H), 7.91 (s, 1H), 7.69-7.55 (m, 2H), 7.48-7.39 (m, 2H), 7.34 (t, J=7.3 Hz, 1H), 6.73 (s, 1H), 4.67 (s, 1H), 4.56 (dt, J=13.5, 6.8 Hz, 1H), 4.39 (d, J=4.8 Hz, 1H), 3.40-3.27 (m, 2H), 3.28-3.16 (m, 1H), 3.12 (s, 1H), 2.12 (dd, J=13.4, 7.0 Hz, 1H), 1.97 (d, J=5.7 Hz, 1H), 1.48 (d, J=6.8 Hz, 6H).

### Example 39: Synthesis of Compound UB-039

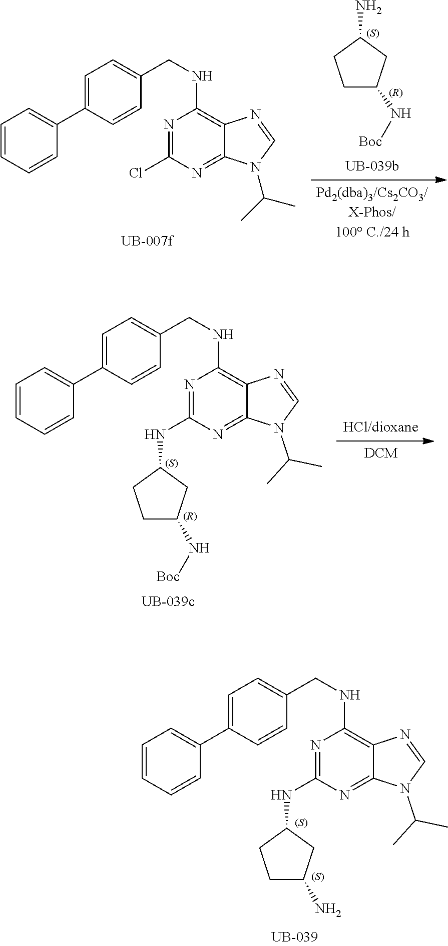

### Step 1: UB-039c

### Tert-butyl ((1R,3S)-3-((6-(([1,1′-biphenyl]-4-ylmethyl)amino)-9-isopropyl-9H-purin-2-yl)amino)cyclo pentyl)carbamate

UB-007f (100 mg, 0.265 mmol), UB-039b (160 mg, 0.8 mmol), Pd₂(dba)₃ (146.6 mg, 0.16 mmol), Cs2CO₃ (172.7 mg, 0.53 mmol), X-phos (380.8 mg, 0.8 mmol) and 1,4-dioxane (10 mL) were added to a reaction flask. The reaction mixture was replaced with argon three times and then heated to 100° C. and stirred overnight. After the reaction was completed, the reaction solution was spin-dried and purified by silica gel column using DCM/MeOH=0-25% (10% MeOH) to obtain a yellow solid compound UB-039c (40 mg, yield 28%). LCMS [M+1]⁺=542.3.

### Step 2: UB-039

### N6-([1,1′-biphenyl]-4-ylmethyl)-N2-((1S,3R)-3-aminocyclopentyl)-9-isopropyl-9H-purin-2,6-diamine

UB-039c (50 mg, 0.09 mmol) was dissolved in 3 mL of THF, and then HCl/dioxane (1 mL) was added. The reaction solution was stirred at room temperature overnight. The reaction solution was purified by silica gel chromatography to obtain a white solid compound UB-039 (3.3 mg, yield 8%). LCMS [M+1]⁺=442.3. ¹H NMR (400 MHz, DMSO-d₆) ¹H NMR (400 MHz,) 6 7.88 (s, 1H), 7.82 (s, 1H), 7.61 (dd, J=13.6, 7.8 Hz, 4H), 7.44 (dd, J=11.9, 5.3 Hz, 4H), 7.34 (t, J=7.3 Hz, 1H), 6.44 (d, J=7.1 Hz, 1H), 4.66 (s, 2H), 4.53 (dt, J=13.4, 6.8 Hz, 1H), 4.18 (d, J=7.0 Hz, 1H), 2.43-2.30 (m, 1H), 2.00 (dd, J=14.8, 7.0 Hz, 1H), 1.89 (s, 2H), 1.65 (d, J=4.8 Hz, 2H), 1.47 (d, J=6.7 Hz, 6H), 1.36-1.28 (m, 2H), 1.25 (s, 2H), 1.22-1.22 (m, 1H).

### Example 40: Synthesis of Compound UB-040

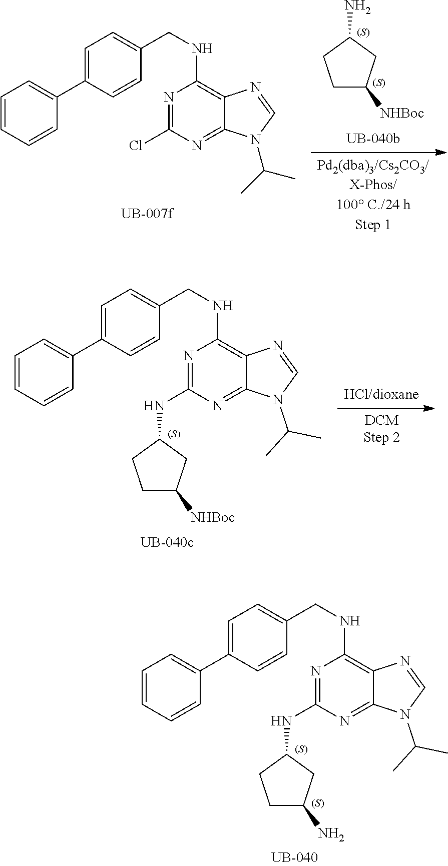

### Step 1: UB-040c

### Tert-butyl ((1S,3S)-3-((6-(([1,1′-biphenyl]-4-ylmethyl)amino)-9-isopropyl-9H-purin-2-yl)amino)cyclo pentyl)carbamate

UB-007f (50 mg, 0.13 mmol), UB-040b (96.1 mg, 0.48 mmol), Pd₂(dba)₃ (18.3 mg, 0.02 mmol), Xphos (38.2 mg, 0.08 mmol) and Cs2CO₃ (156.4 mg, 0.48 mmol), and 1,4-dioxane (10 mL) were added to a reaction flask. The reaction mixture was replaced with argon three times and then heated to 100° C. and stirred overnight. Then the reaction mixture was stirred at 100° C. for 24 hours. The reaction mixture was diluted with water (30 mL), and then extracted with ethyl acetate (30 mL*3). The organic phase was separated, washed with brine (50 mL) and dried over anhydrous sodium sulfate, then concentrated and purified by chromatography (dichloromethane:dichloromethane/methanol 10/1=75:25) to obtain the product a yellow solid compound (UB-040c, 38 mg, yield: 21.9%). LCMS [M+1]⁺=542.3.

### Step 2: UB-040

### N⁶—([1,1′-biphenyl]-4-ylmethyl)-N2-((1S,3S)-3-aminocyclopentyl)-9-isopropyl-9H-purin-2,6-diamine

To a 100 mL bottom flask were added UB-040c (37.9 mg, 0.07 mmol), a solution of hydrochloric acid in 1,4-dioxane (2 mL), and dichloromethane (4.0 mL) at room temperature. Then the solution was stirred at room temperature for 2 hours. The reaction mixture was filtered, and the filter cake was washed with dichloromethane (5 mL), neutralized with saturated aqueous sodium bicarbonate solution, and extracted with dichloromethane (3*50 mL). The combined organic phase was washed with brine, dried over anhydrous sodium sulfate and concentrated under reduced pressure, then added with methanol and water and freeze-dried to obtain a yellow solid product (UB-040, 13.9 mg, yield: 44.8%). LCMS [M+1]⁺=428.2. ¹H NMR (400 MHz, DMSO-d₆) δ 7.78 (s, 1H), 7.64-7.61 (m, 2H), 7.58 (d, J=8.2 Hz, 2H), 7.47-7.40 (m, 5H), 7.33 (t, J=7.3 Hz, 1H), 6.19 (d, J=7.0 Hz, 1H), 4.67 (s, 2H), 4.53 (dt, J=13.3, 6.6 Hz, 1H), 4.31 (dd, J=14.1, 7.1 Hz, 1H), 2.07-2.00 (m, 1H), 1.90-1.82 (m, 1H), 1.68 (dd, J=13.1, 6.6 Hz, 1H), 1.64-1.56 (m, 1H), 1.47 (d, J=6.7 Hz, 6H), 1.43-1.34 (m, 2H), 1.25-1.14 (m, 2H).

### Example 41: Synthesis of Compound UB-041

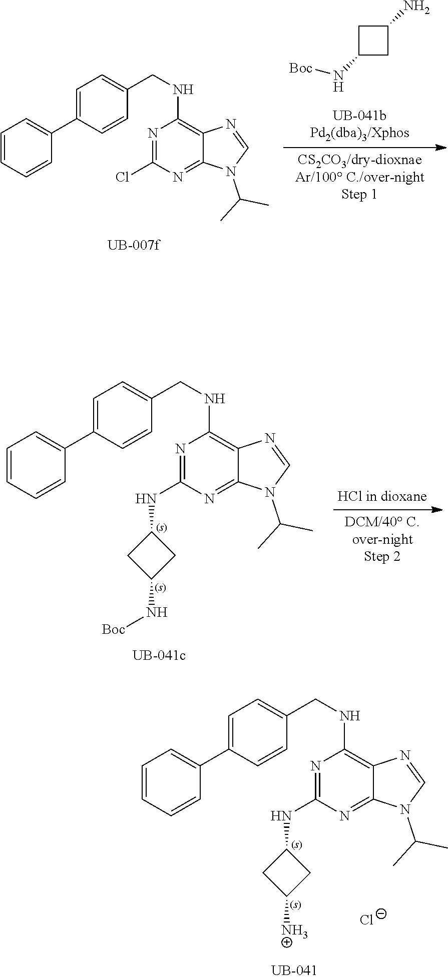 [SMILES: CC(C)n1cnc2c(NCc3ccc(-c4ccccc4)cc3)nc(Cl)nc21 ; CC(C)(C)OC(=O)N[C@H]1C[C@@H](N)C1 ; CC(C)n1cnc2c(NCc3ccc(-c4ccccc4)cc3)nc(N[C@H]3C[C@@H](NC(=O)OC(C)(C)C)C3)nc21 ; CC(C)n1cnc2c(NCc3ccc(-c4ccccc4)cc3)nc(N[C@H]3C[C@@H]([NH3+])C3)nc21 ; [Cl-]]

### Step 1: UB-041c

### Tert-butyl 2-((1s,3s)-3-((6-(([1,1′-biphenyl]-4-ylmethyl)amino)-9-isopropyl-9H-purin-2-yl)amino)

Compound UB-041b (158 mg, 0.85 mmol), compound UB-007f (80 mg, 0.21 mmol), cesium carbonate (207 mg, 0.64 mmol), Pd₂(dba)₃ (25 mg, 0.02 mmol) and Xphos (51 mg, 0.11 mmol) were dissolved in (5 mL) ultra-dry dioxane and the resulting mixture was reacted at 100° C. overnight. After the reaction was completed, the reaction solution was cooled to room temperature, concentrated and purified by silica gel column (EA/PE=50%) to obtain the compound (UB-041c, 30 mg, 5% yield) as a yellow solid. LCMS [M+1]⁺=528.3.

### Step 2: UB-041

### (1s,3s)-3-((6-(([1,1′-biphenyl]-4-ylmethyl)amino)-9-isopropyl-9H-purin-2-yl)amino)cyclobutan-1-ammonium chloride

Compound UB-041c (30 mg, 0.06 mmol) was dissolved in dichloromethane (2 mL), and hydrochloric acid in 1,4-dioxane (2 mmol, 0.5 mL) was added. The reaction was stirred at 40° C. for 4 hours. After the reaction was completed, the reaction solution was added with saturated sodium bicarbonate solution to adjust the pH to 8-9, and extracted with DCM (40 mL*3). The organic phases were combined, washed with saturated salt water, and dried over anhydrous sodium sulfate. The extract was concentrated and lyophilized to give the desired compound UB-041 (14.9 mg, 55% yield) as a white solid. LCMS [M+1]⁺=428.3. ¹H NMR (400 MHz, DMSO-d₆) δ 9.60 (d, J=117.0 Hz, 1H), 8.73 (s, 1H), 8.31 (s, 3H), 7.64 (d, J=7.7 Hz, 4H), 7.55-7.44 (m, 4H), 7.36 (t, J=7.3 Hz, 1H), 5.30 (d, J=16.8 Hz, 1H), 4.86-4.62 (m, 3H), 4.13 (s, 1H), 2.67 (s, 2H), 2.13 (d, J=7.7 Hz, 2H), 1.53 (d, J=6.3 Hz, 6H), 1.23 (s, 1H).

### Example 42: Synthesis of Compound UB-042

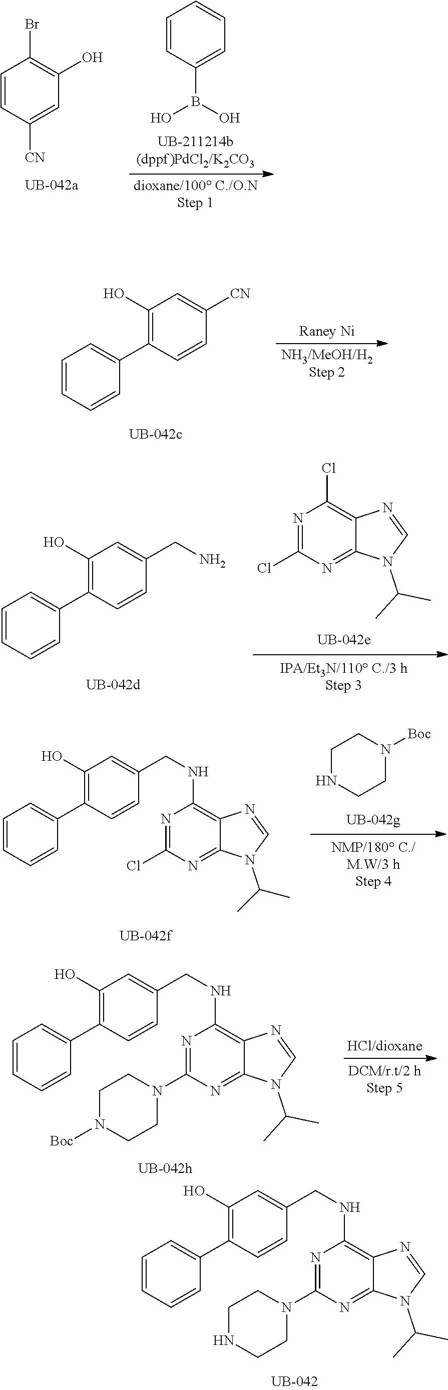

### Step 1: UB-042c

### 2-Hydroxy-[1,1′-biphenyl]-4-carbonitrile

To a 100 mL round-bottom flask were added in sequence compound UB-042a (1.55 g, 7.57 mmol), UB-042b (1.85 g, 15.14 mmol), PPd(dppf)Cl₂ (550 mg, 0.757 mmol), K₂CO₃ (3.13 g, 22.71 mmol), 1,4-dioxane (45 mL) and water. The resulting mixture was replaced with argon three times and then heated to 100° C. and reacted overnight. After the reaction was completed, the mixture was concentrated under reduced pressure. The residue was separated by column chromatography (eluent: DCM:PE:EA=100/0-30/70) to obtain a light yellow solid compound UB-042c (1.1 g, yield 78%). LCMS [M+H]⁺=196.1.

### Step 2: UB-042d

### 4-(Aminomethyl)-[1,1′-biphenyl]-2-ol

UB-042c (1.1 g, 5.64 mmol), Raney nickel (110 mg), and amine methanol (10 mL) were added to a 100 mL round-bottom flask. The resulting mixture was replaced with hydrogen three times and reacted at room temperature for 1 hour. After the reaction was completed, the mixture was filtered through diatomite, and the filtrate was concentrated under reduced pressure. The residue was separated by column chromatography (eluent: DCM/(10% MeOH in DCM)═100/0-50/50) to obtain a white solid compound UB-042d (840 mg, yield 63%). LCMS [M+H]⁺=200.1.

### Step 3: UB-042f

### 4-((2-chloro-9-isopropyl-9H-purin-6-yl)amino)methyl)-[1,1′-biphenyl]-2-ol

UB-042d (840 mg, 4.22 mmol), UB-042e (1.02 g, 4.43 mmol), triethylamine (1.28 g, 12.66 mmol), and isopropanol (15 mL) were added to a 100 mL round-bottom flask. The resulting mixture was reacted at 110° C. for 3 hours. After the reaction was completed, the mixture was cooled to room temperature. The precipitated solid was filtered and the filter cake was washed with ethanol and water in a ratio of 10:1 to obtain a white solid compound UB-042f (1.3 g, yield 78%). LCMS [M+H]⁺=394.1.

### Step 4: UB-042h

### Tert-butyl 4-(6-((2-hydroxy-[1,1′-biphenyl]-4-yl)methyl)amino)-9-isopropyl-9H-purin-2-yl)piperazin-1-carboxylate

UB-042f (1.0 g, 2.54 mmol), UB-042g (2.36 g, 12.72 mmol), DIPEA (2.62 g, 20.35 mmol), and NMP (8 mL) were added to a 20 mL microwave tube. The resulting mixture was reacted under microwave condition at 180° C. for 3 hours. After the reaction finished, 30 mL of water was added. The solid precipitated and was filtered. The filter cake was separated by column chromatography (eluent: DCM/(10% MeOH in DCM)═100/0-30/70) to obtain a white solid compound UB-042h (900 mg, yield 65%). LCMS [M+H]⁺=544.3

### Step 5: UB-042

### 4-(9-isopropyl-2-(piperazin-1-yl)-9H-purin-6-yl)amino)methyl)-[1,1′-biphenyl]-2-ol

UB-042h (60 mg, 0.11 mmol), dichloromethane (3 mL) and hydrochloric acid in dioxane (2 mL, 8 mmol) were added to a 50 mL round-bottom flask, and the resulting mixture was reacted at room temperature for 4 hours. The reaction was quenched with saturated sodium bicarbonate solution until the pH was neutral, and then extracted with dichloromethane, and the organic phase was concentrated under reduced pressure and the residue was subjected to preparative chromatography to obtain a white solid compound UB-042 (14 mg, yield 29%). LCMS [M+1]⁺=444.1. ¹H NMR (400 MHz, DMSO-d₆) δ 9.42 (s, 1H), 8.85 (s, 2H), 8.16 (d, J=14.5 Hz, 1H), 7.93 (s, 1H), 7.56-7.44 (m, 2H), 7.36 (t, J=7.6 Hz, 2H), 7.26 (t, J=7.3 Hz, 1H), 7.16 (d, J=7.8 Hz, 1H), 6.94 (s, 1H), 6.87 (d, J=7.9 Hz, 1H), 4.73-4.32 (m, 3H), 3.87 (d, J=4.8 Hz, 4H), 3.08 (s, 4H), 1.48 (d, J=6.8 Hz, 6H).

### Example 43: Synthesis of Compound UB-043

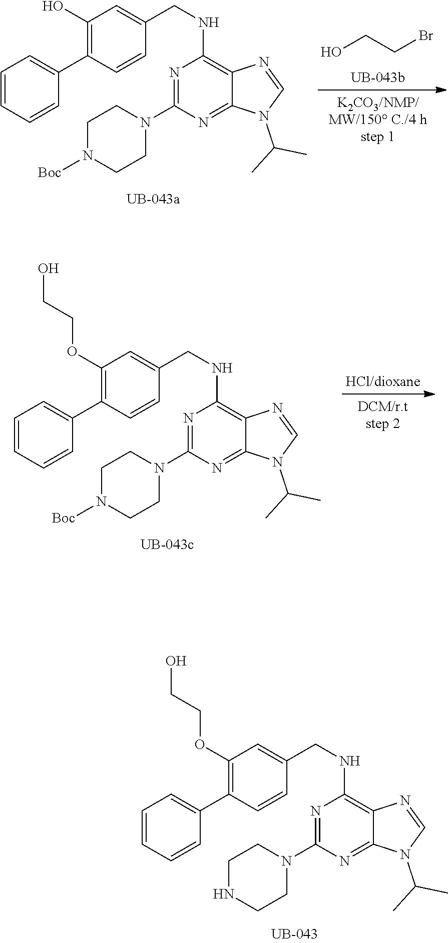

### Step 1: UB-043c

### Tert-butyl 4-(6-(((2-hydroxyethoxy)-[1,1′-biphenyl]-4-yl)methyl)amino)-9-isopropyl-9H-purin-2-yl)piperazin-1-carboxylate

Compound UB-043a (100 mg, 0.18 mmol), compound UB-043b (112.6 mg, 0.90 mmol), K₂CO₃ (124.2 mg, 0.90 mmol) and 5 mL of N-methylpyrrolidone were added to a microwave tube, and the reaction solution was heated to 150° C. in the microwave tube for 4 hours. The reaction solution was poured into water and filtered to obtain a filter cake. The filtrate was extracted twice with ethyl acetate. The organic phase and the filter cake were combined. After removing the solvent, the concentrate was purified by flash chromatography (methanol:dichloromethane (1:10)/dichloromethane=0-70%) to obtain a white solid compound UB-043c (73.0 mg, yield 66.7%). LCMS [M+1]⁺=588.3.

### Step 2: UB-043

### 2-((4-(((9-isopropyl-2-(piperazin-1-yl)-9H-purin-6-yl)amino)methyl)-[1,1′-biphenyl]-2-yl)oxy)ethanol

Compound UB-043c (73.0 mg, 0.12 mmol) was dissolved in 4 mL of dichloromethane, and hydrochloric acid in dioxane (1 mL) was added, and the mixture was stirred at room temperature for 3 hours. The reaction solution was neutralized with sodium bicarbonate solution, and extracted with dichloromethane; the organic phase was collected and dried, and after removing the solvent, the concentrate was purified by flash chromatography ((methanol:dichloromethane (1:10)/dichloromethane=0-100%) to obtain a white solid compound UB-043 (29.7 mg, yield 49.0%). LCMS [M+1]⁺=488.3. ¹H NMR (400 MHz, DMSO-d₆) δ 7.97 (s, 1H), 7.85 (s, 1H), 7.56-7.48 (m, 2H), 7.36 (t, J=7.5 Hz, 2H), 7.31-7.24 (m, 1H), 7.24-7.15 (m, 2H), 7.02 (d, J=7.7 Hz, 1H), 4.76 (s, 1H), 4.57 (dt, J=13.5, 6.7 Hz, 3H), 3.98 (t, J=5.2 Hz, 2H), 3.62 (dd, J=12.8, 8.1 Hz, 6H), 2.71 (dd, J=8.3, 3.4 Hz, 4H), 1.46 (d, J=6.8 Hz, 6H).

### Example 44: Synthesis of Compound UB-044

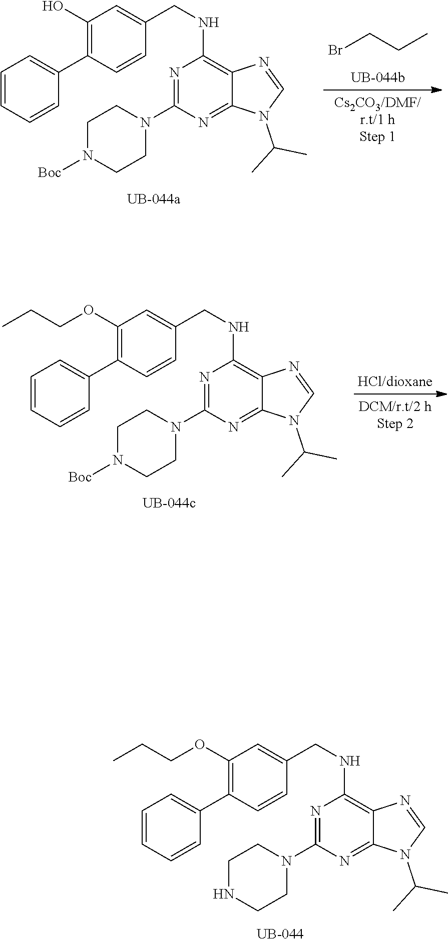

### Step 1: UB-044c

### Tert-butyl (S)-(1-(6-([1,1′-biphenyl]-4-ylmethyl)amino)-9-isopropyl-9H-purin-2-yl) pyrrolidin-3-yl)carbamate

UB-044a (80 mg, 0.212 mmol), UB-044b (197 mg, 1.06 mmol), DIPEA (218 mg, 1.69 mmol), and NMP (3 mL) were added to a 10 mL microwave tube. The resulting mixture was reacted under microwave at 180° C. for 3 hours. After the reaction finished, 30 mL of water was added. The solid precipitated and was filtered. The filter cake was separated by column chromatography (eluent: DCM/(10% MeOH in DCM)═100/0-30/70) to obtain a white solid compound UB-044c (60 mg, yield 54%). LCMS [M+H]⁺=528.3

### Step 2: UB-044

### (S)—N-([1,1′-biphenyl]-4-ylmethyl)-2-(3-aminopyrrolidin-1-yl)-9-isopropyl-9H-purin-6-amine

UB-044c (60 mg, 0.11 mmol), dichloromethane (3 mL) and hydrochloric acid in dioxane (2 mL, 8 mmol) were added to a 50 mL round-bottom flask, and the resulting mixture was reacted at room temperature for 4 hours. The reaction was quenched with saturated sodium bicarbonate solution until the pH was neutral, and then extracted with dichloromethane, and the organic phase was concentrated under reduced pressure. The residue was separated by column chromatography (eluent: DCM/(10% MeOH in DCM)═100/0-90/10) to obtain a white solid compound UB-044 (14 mg, yield 29%). LCMS [M+1]⁺=486.2. ¹H NMR (400 MHz, DMSO-d₆) δ 8.24 (s, 1H), 8.04 (s, 1H), 7.87 (s, 1H), 7.46 (d, J=7.2 Hz, 2H), 7.37 (t, J=7.6 Hz, 2H), 7.28 (t, J=7.3 Hz, 1H), 7.20 (d, J=7.7 Hz, 1H), 7.15 (s, 1H), 7.01 (d, J=7.0 Hz, 1H), 4.57 (dd, J=13.5, 6.8 Hz, 3H), 3.89 (t, J=6.4 Hz, 2H), 3.70 (s, 5H), 2.83 (s, 4H), 1.64 (dd, J=13.9, 6.7 Hz, 2H), 1.47 (d, J=6.8 Hz, 6H), 0.89 (t, J=7.4 Hz, 3H).

### Example 45: Synthesis of Compound UB-045

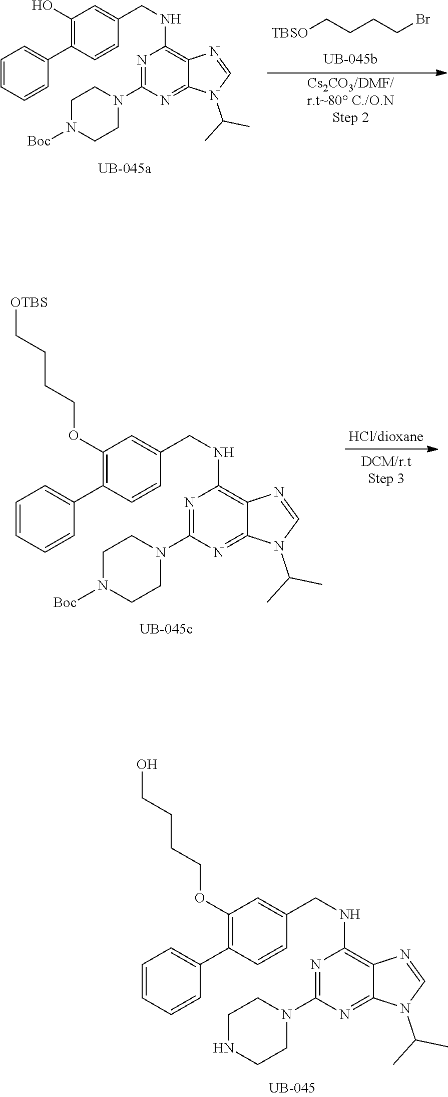 [SMILES: CC(C)n1cnc2c(NCc3ccc(-c4ccccc4)c(O)c3)nc(N3CCN(C(=O)OC(C)(C)C)CC3)nc21 ; CC(C)(C)[Si](C)(C)OCCCCBr ; CC(C)n1cnc2c(NCc3ccc(-c4ccccc4)c(OCCCCO[Si](C)(C)C(C)(C)C)c3)nc(N3CCN(C(=O)OC(C)(C)C)CC3)nc21 ; CC(C)n1cnc2c(NCc3ccc(-c4ccccc4)c(OCCCCO)c3)nc(N3CCNCC3)nc21]

### Step 1: UB-045c

### Tert-Butyl 4-(6-(((2-(4-((tert-butyldimethylsilyl)oxy)butoxy)-[1,1′-biphenyl]-4-yl)methyl)amino)-9-iso propyl-9H-purin-2-yl)piperazin-1-carboxylate

Compound UB-045a (80 mg, 0.147 mmol), cesium carbonate (96 mg, 0.294 mmol) and 5 mL of N,N-dimethylformamide were added to a 100 mL reaction bottle. The reaction solution was stirred until it turned slightly yellow, and then compound UB-045b (80 mg, 0.294 mmol) was added. The reaction solution was heated to 80° C. for 18 hours. After cooling to room temperature and removing the solvent, the concentrate was purified by flash chromatography (ethyl acetate/petroleum ether=0-50%) to give a white solid compound 1225c (110.0 mg, yield 97%). LCMS [M+1]⁺=616.4.

### Step 2: UB-045

### 4-((4-(((9-isopropyl-2-(piperazin-1-yl)-9H-purin-6-yl)amino)methyl)-[1,1′-biphenyl]-2-yl)oxy)butan-1-ol

Compound UB-045c (110.0 mg, 0.179 mmol) was dissolved in 4 mL of dichloromethane, and hydrochloric acid in dioxane (1 mL) was added, and the mixture was stirred at room temperature for 3 hours. The reaction solution was neutralized with sodium bicarbonate solution, and extracted with dichloromethane; the organic phase was collected and dried, and after removing the solvent, the concentrate was purified by flash chromatography ((methanol:dichloromethane (1:10)/dichloromethane=0-100%) to obtain a white solid compound UB-045 (41.7 mg, yield 48.1%). LCMS [M+1]⁺=516.3. ¹H NMR (400 MHz, DMSO-d₆) δ 8.14 (s, 1H), 7.91 (s, 1H), 7.48-7.42 (m, 2H), 7.36 (dd, J=15.2, 7.4 Hz, 2H), 7.28 (t, J=7.3 Hz, 1H), 7.19 (dd, J=14.0, 7.2 Hz, 2H), 7.01 (d, J=7.0 Hz, 1H), 4.58 (dd, J=13.5, 6.7 Hz, 3H), 3.94 (t, J=6.5 Hz, 2H), 3.82 (d, J=3.8 Hz, 4H), 3.39 (d, J=6.4 Hz, 2H), 3.00 (s, 4H), 1.66 (dd, J=14.6, 6.7 Hz, 2H), 1.48 (t, J=6.8 Hz, 8H).

### Example 46: Synthesis of Compound UB-046

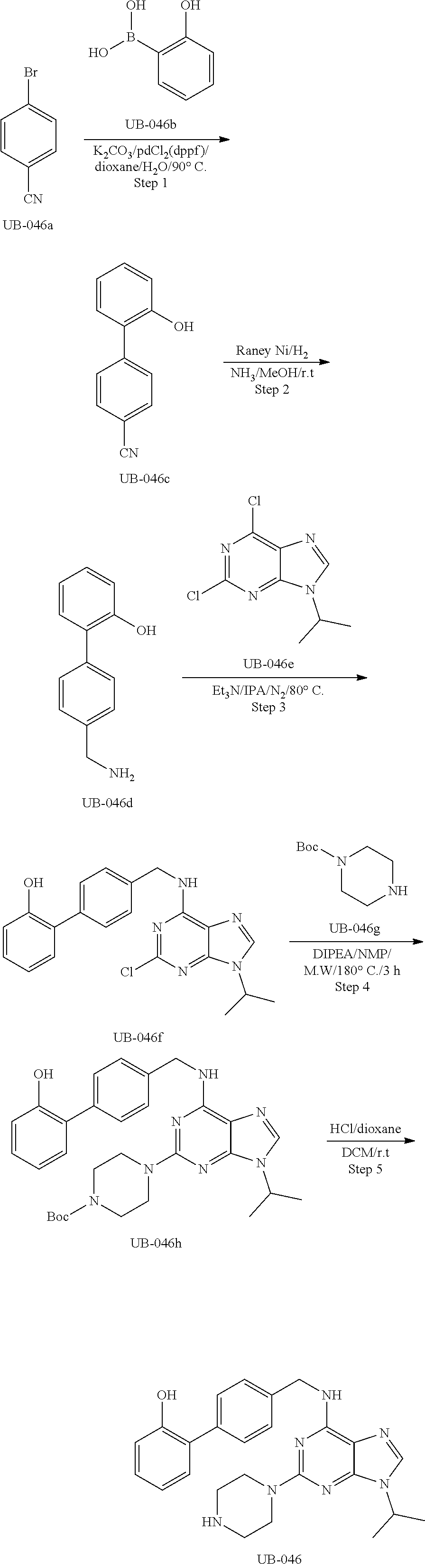

### Step 1: UB-046c

### 2′-Hydroxy-[1, 1′-biphenyl]-4-carbonitrile

To a solution of UB-046a (3.0 g, 16.5 mmol) and UB-046b (3.4 g, 24.7 mmol) in 1,4-dioxane (60 mL) and H₂O (20 mL) were added (dppf)PdCl₂ (600 mg, 0.82 mmol), and K₂CO₃ (6.8 g, 49.4 mmol) under N2. The mixture was stirred at reflux for 4 hours and the mixture gradually became cloudy. The reaction mixture was concentrated and the residue was purified by silica gel column chromatography [eluent: PE/EA=0-40%] to give the yellow solid compound (UB-046c, 3.26 g, yield 100%). LCMS [M+1]⁺=177.1

### Step 2: UB-046d

### 4′-(Aminomethyl)-[1,1′-biphenyl]-2-01

UB-046c (2.8 g, 14.36 mmol), Raney Ni (280 mg), and NH₃ in methanol (15 mL) were added under H2. The mixture was stirred at room temperature overnight. The mixture was filtered through diatomite, and the filtrate was concentrated and purified by silica gel column chromatography [eluent: DCM/10% MeOH/DCM=0-50%] to give a white solid compound (UB-046d, 1.4 g, yield 49%). LCMS [M+1]⁺=200.1

### Step 3: UB-046f

### 4′-(((2-chloro-9-isopropyl-9H-purin-6-yl)amino)methyl)-[1,1′-biphenyl]-2-ol

UB-046d (1090 mg, 5.48 mmol), UB-046e (1290 mg, 5.58 mmol), Et₃N (1660 mg, 16.44 mmol) and isopropanol (20 mL) were added to a reaction flask. The mixture was stirred at 80° C. for 4 hours. The mixture was concentrated and purified by silica gel column chromatography [eluent: DCM/10% MeOH/DCM=0-50%] to give a yellow solid compound (1218f, 1.8g, yield 83.4%). LCMS [M+1]⁺=394.2

### Step 4: UB-046h

### Tert-butyl 4-(6-(((2′-hydroxy-[1,1′-biphenyl]-4-yl)methyl)amino)-9-isopropyl-9H-purin-2-yl)piperazin-1-carboxylate

UB-046f (1500 mg, 3.807 mmol), UB-046g (3.54 g, 19.03 mmol), DIPEA (3.93 g, 30.4 mmol) and NMP (6 mL) were added to a microwave tube. The mixture was stirred at 180° C. under microwave condition for 3 hours. The reaction mixture was poured into H₂O. The resulting precipitate was collected by filtration, and the filtrate was extracted with EA three times. The organic phases were combined, and the filtrate was concentrated and purified by silica gel column chromatography [eluent: DCM/10% MeOH/DCM)═0-20%] to obtain a white solid compound (UB-046h, 1.11 g, yield 53.2%). LCMS [M+1]⁺=544.3

### Step 5: UB-046

### 4′-(((9-isopropyl-2-(piperazin-1-yl)-9H-purin-6-yl)amino)methyl)-[1,1′-biphenyl]-2-ol

To a solution of UB-046h (50 mg, 0.09 mmol) in 3 mL of DCM was added a solution of HCl in dioxane (1 mL). The mixture was stirred at room temperature for 2 hours. The reaction mixture was quenched with NaHCO₃ and extracted with DCM (10 mL*3). The organic phase was separated, washed with brine (10 mL*3) and dried over Na₂SO₄, then concentrated and purified by flash chromatography [elute: DCM:10% MeOH/DCM=0-100%] to obtain a white solid compound (UB-046, 13.5 mg, yield 33.9%) LCMS [M+1]⁺=444.3. ¹H NMR (400 MHz, DMSO-d₆) δ 9.44 (s, 1H), 7.96 (s, 1H), 7.84 (s, 1H), 7.44 (d, J=8.2 Hz, 2H), 7.37 (d, J=8.1 Hz, 2H), 7.20 (dd, J=7.6, 1.6 Hz, 1H), 7.15-7.09 (m, 1H), 6.91 (d, J=8.0 Hz, 1H), 6.84 (t, J=7.4 Hz, 1H), 4.69-4.47 (m, 3H), 3.61 (d, J=5.0 Hz, 4H), 2.71 (d, J=4.5 Hz, 4H), 1.46 (d, J=6.7 Hz, 6H).

### Example 47: Synthesis of Compound UB-047

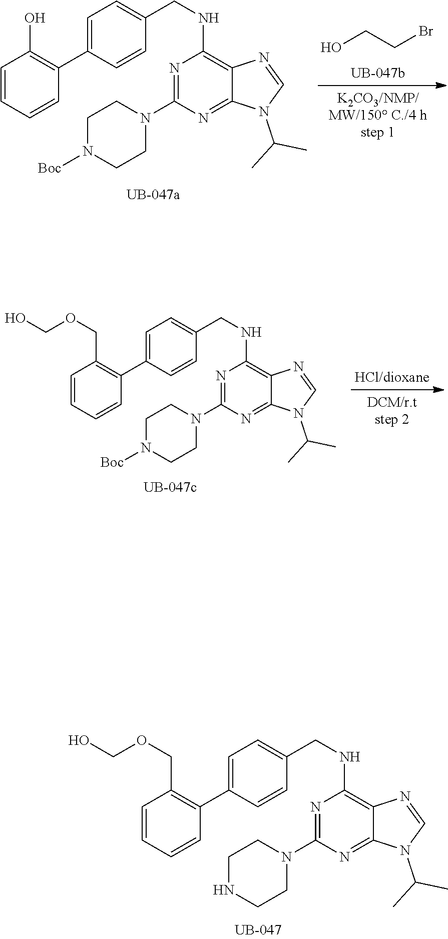

### Step 1: UB-047c

### Tert-butyl

### 4-(6-(((2′-(2-hydroxyethoxy)-[1,1′-biphenyl]-4-yl)methyl)amino)-9-isopropyl-9H-purin-2-yl)piperazin-1-carboxylate

A solution of UB-047a (80 mg, 0.147 mmol), UB-047b (92 mg, 0.737 mmol), K₂CO₃ (102 mg, 0.737 mmol) in NMP (3 mL) was added to a microwave tube. The mixture was stirred at 150° C. under microwave condition for 4 hours. The reaction mixture was poured into H₂O. The resulting precipitate was collected by filtration, and the filtrate was extracted with EA three times. The organic phases were combined, and the filtrate was concentrated and purified by silica gel column chromatography [eluent: DCM/10% MeOH/DCM)═0-70%] to obtain a white solid compound (UB-047c, 60 mg, yield 69.5%). LCMS [M+1]⁺=588.3

### Step 2: UB-047

### 2-((4′-(((9-isopropyl-2-(piperazin-1-yl)-9H-purin-6-yl)amino)methyl)-[1,1′-biphenyl]-2-yl)oxy)ethan-1-ol

A solution of UB-047c (60 mg, 0.102 mmol) and 3 mL of DCM was added to a reaction flask, and HCl in dioxane (2 mL) was added. The mixture was stirred at room temperature for 2 hours. The reaction mixture was quenched with NaHCO₃ and extracted with DCM (10 mL*3). The organic phase was separated, washed with brine (10 mL*3) and dried over Na₂SO₄, then concentrated and purified by flash chromatography, using DCM:10% MeOH/DCM=0-100% as eluent, to obtain a light yellow solid compound (UB-047, 39 mg, yield 78.5%) LCMS [M+1]⁺=488.1. ¹H NMR (400 MHz, DMSO-d₆) δ 8.04 (d, J=19.2 Hz, 1H), 7.86 (s, 1H), 7.48 (d, J=8.2 Hz, 2H), 7.37 (d, J=8.2 Hz, 2H), 7.29 (dd, J=13.3, 4.8 Hz, 2H), 7.09 (d, J=8.0 Hz, 1H), 6.99 (t, J=7.0 Hz, 1H), 4.58 (td, J=13.8, 7.1 Hz, 3H), 4.01 (t, J=5.1 Hz, 2H), 3.68-3.62 (m, 6H), 2.81-2.72 (m, 4H), 1.47 (d, J=6.8 Hz, 6H).

### Example 48: Synthesis of Compound UB-048

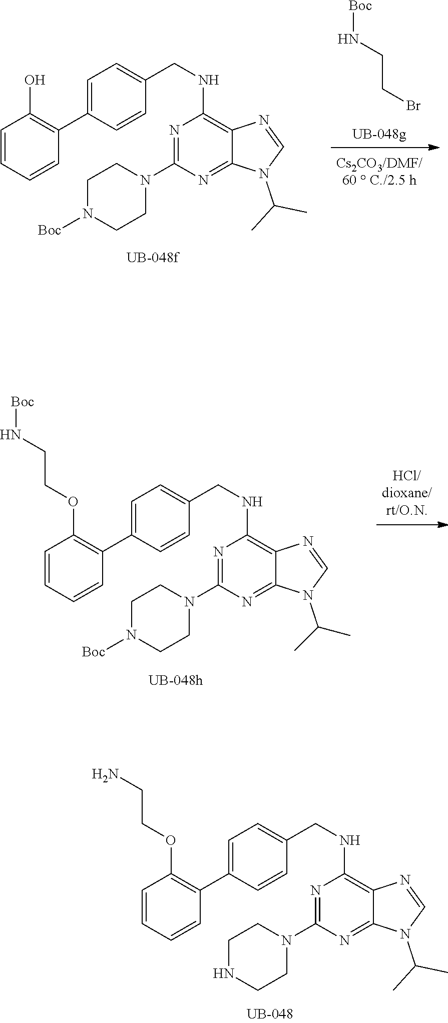 [SMILES: CC(C)n1cnc2c(NCc3ccc(-c4ccccc4O)cc3)nc(N3CCN(C(=O)OC(C)(C)C)CC3)nc21 ; CC(C)(C)OC(=O)NCCBr ; CC(C)n1cnc2c(NCc3ccc(-c4ccccc4OCCNC(=O)OC(C)(C)C)cc3)nc(N3CCN(C(=O)OC(C)(C)C)CC3)nc21 ; CC(C)n1cnc2c(NCc3ccc(-c4ccccc4OCCN)cc3)nc(N3CCNCC3)nc21]

### Step 1: UB-048h

A mixture of compound UB-048f (80 mg, 0.15 mmol) and Cs2CO₃ (384 mg, 1.18 mmol) in DMF (3 mL) was stirred at room temperature for 5 minutes, and then the mixture was added with compound UB-048g (264 mg, 1.18 mmol) and stirred at 60° C. for 2.5 hours. The mixture was cooled to room temperature, concentrated under reduced pressure and purified by silica gel column chromatography [dichloromethane (dichloromethane:methanol=10:1)═0-21%] to give the product UB-048h (64 mg, yield: 63%) as a white solid. LCMS [M+1]⁺=687.2.

### Step 2: UB-048

To a mixture of compound UB-048h (64 mg, 0.09 mmol) in CH₂Cl₂ (3 mL) was added HCl/dioxane (4 N, 1.5 mL). The reaction mixture was stirred at 25° C. for 17 hours. The reaction mixture was concentrated and adjusted to pH=8-9 with saturated aqueous Na₂CO₃ solution. After extraction with CH₂C₂ (20 mL*3), the combined organic layer was washed with brine, and dried over anhydrous Na₂SO₄, and then the mixture was concentrated under reduced pressure to obtain product UB-048 (29.3 mg, yield: 65%) as a white solid. LCMS [M+1]⁺=487.3. ¹H NMR (400 MHz, DMSO-d₆) δ 7.97 (s, 1H), 7.86-7.81 (s, 1H), 7.44 (d, J=8.2 Hz, 2H), 7.38 (d, J=8.2 Hz, 2H), 7.27 (dt, J=7.7, 4.5 Hz, 2H), 7.08 (d, J=7.9 Hz, 1H), 7.00 (t, J=7.1 Hz, 1H), 4.57 (td, J=13.6, 6.8 Hz, 3H), 3.93 (t, J=5.6 Hz, 2H), 3.70 (d, J=24.2 Hz, 1H), 3.65-3.55 (m, 4H), 2.81 (t, J=5.6 Hz, 2H), 2.70 (s, 4H), 1.46 (d, J=6.8 Hz, 6H).

### Example 49: Synthesis of Compound UB-049

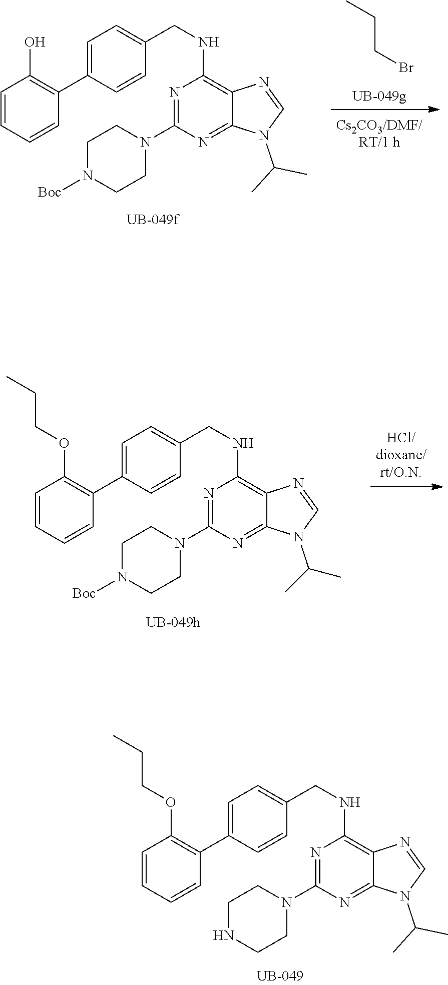

### Step 1: UB-049h

A mixture of compound UB-049f (100 mg, 0.18 mmol) and Cs2CO₃ (180 mg, 0.55 mmol) in DMF (3 mL) was stirred at room temperature for 5 minutes, and then the mixture was added with compound UB-049g (86 mg, 0.70 mmol) and stirred at room temperature for 1 hour. H₂O (20 mL) was added to the reaction mixture, and then the aqueous layer was extracted with dichloromethane (30 mL×4). The organic layers were combined and dried over anhydrous Na₂SO₄, and then the mixture was concentrated under reduced pressure to give the crude product UB-049h (89 mg, yield: 83%) as a light yellow liquid, which was directly used in the next step without purification. LCMS [M+1]=586.4

### Step 2: UB-049

To a mixture of compound UB-049h (89 mg, 0.15 mmol) in CH₂Cl₂ (3 mL) was added HCl/dioxane (4 N, 1.5 mL). The reaction mixture was stirred at 25° C. for 17 hours. The reaction mixture was concentrated and adjusted to pH=8-9 with saturated aqueous Na₂CO₃ solution. After extraction with CH₂Cl₂ (20 mL*3), the combined organic layer was washed with brine, and dried over anhydrous Na₂SO₄, and then the mixture was concentrated under reduced pressure to obtain product UB-049 (54 mg, yield: 70%) as a white solid. LCMS [M+1]⁺=486.3. ¹H NMR (400 MHz, DMSO-d₆) δ 7.97 (s, 1H), 7.84 (s, 2H), 7.42 (d, J=8.2 Hz, 3H), 7.37 (d, J=8.2 Hz, 3H), 7.32-7.23 (m, 3H), 7.06 (d, J=8.1 Hz, 1H), 6.98 (t, J=7.3 Hz, 2H), 4.68-4.50 (m, 4H), 3.92 (t, J=6.3 Hz, 3H), 3.65-3.54 (m, 6H), 2.70 (s, 6H), 1.65 (dd, J=13.8, 6.6 Hz, 3H), 1.46 (d, J=6.8 Hz, 9H), 0.91 (t, J=7.4 Hz, 5H).

### Example 50: Synthesis of Compound UB-050

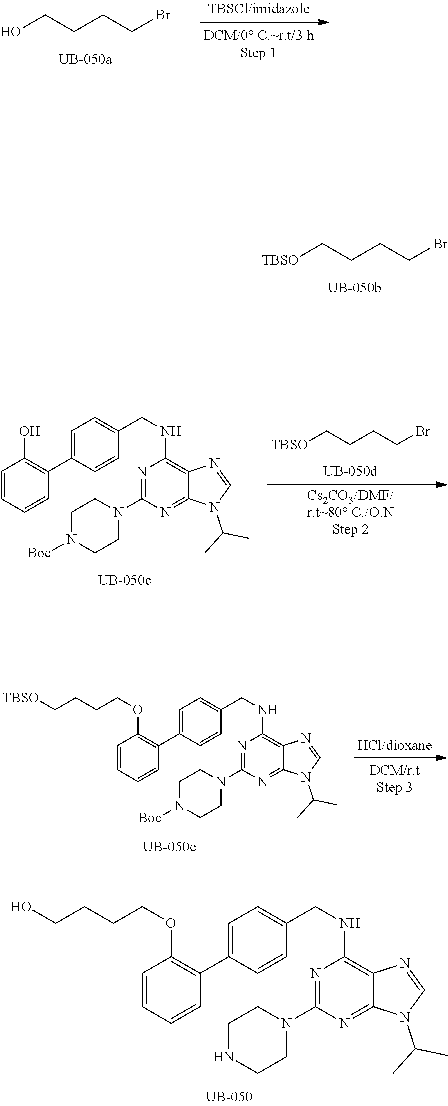 [SMILES: OCCCCBr ; CC(C)(C)[Si](C)(C)OCCCCBr ; CC(C)n1cnc2c(NCc3ccc(-c4ccccc4O)cc3)nc(N3CCN(C(=O)OC(C)(C)C)CC3)nc21 ; CC(C)(C)[Si](C)(C)OCCCCBr ; CC(C)n1cnc2c(NCc3ccc(-c4ccccc4OCCCCO[Si](C)(C)C(C)(C)C)cc3)nc(N3CCN(C(=O)OC(C)(C)C)CC3)nc21 ; CC(C)n1cnc2c(NCc3ccc(-c4ccccc4OCCCCO)cc3)nc(N3CCNCC3)nc21]

### Step 1: UB-050b

### (4-Bromobutoxy)(tert-butyl)dimethylsilane

A solution of UB-050a (500 mg, 3.268 mmol) and DCM (10 mL) was added to a reaction flask under an ice bath, and imidazole (666 mg, 9.8 mmol) was added, and the mixture was stirred for 10 minutes. TBSCl (738 mg, 4.9 mmol) was added slowly and the mixture was stirred for 3 hours. DCM was concentrated, and the residue was diluted with 30 mL of EA, and washed with 10% NaHCO₃ solution and brine (30×2 mL). The organic solvent was dried over Na₂SO₄ and evaporated in vacuo to obtain a colorless oil compound (UB-050b, 620 mg, yield 71%), which was directly used in the next step.

### Step 2: UB-050e

### Tert-Butyl

### 4-(6-(((2′-(4-((tert-butyldimethylsilyl)oxy)butoxy)-[1,1′-biphenyl]-4-yl)methyl)amino)-9-isopropyl-9H-purin-2-yl)piperazin-1-carboxylate

A solution of UB-050c (80 mg, 0.147 mmol) and Cs2CO₃ (96 mg, 0.294 mmol) and DMF (5 mL) was added to a reaction flask and replaced with nitrogen three times. The reaction solution turned yellow after stirring at room temperature. Then UB-050d (80 mg, 0.294 mmol) was added and the reaction solution was heated at 80° C. for 18 hours. The reaction solution was then cooled to room temperature and filtered. The solvent was concentrated and the obtained residue was purified by silica gel column chromatography [eluent: PE/EA=0-50%] to obtain a light yellow solid compound (UB-050e, 110 mg, yield 97%). LCMS [M+1]⁺=616.4

### Step 3: UB-050

### 4-((4′-(((9-isopropyl-2-(piperazin-1-yl)-9H-purin-6-yl)amino)methyl)-[1,1′-biphenyl]-2-yl)oxy)butan-1-ol

UB-050e (100 mg, 0.162 mmol) and 8 mL of DCM solution were added to a reaction flask, and HCl in dioxane (3 mL) was added. The mixture was stirred at room temperature overnight. The reaction mixture was quenched with NaHCO₃ (5 mL) and extracted with DCM (10 mL*3). The organic phase was separated, washed with brine (10 mL*3) and dried over Na₂SO₄, then concentrated and purified by flash chromatography, using DCM: 10% MeOH/DCM=0-100% as eluent, to obtain a white solid compound (UB-050, 14 mg, yield 16.7%). LCMS [M+1]⁺=516.3. ¹H NMR (400 MHz, DMSO-d₆) δ 7.97 (s, 1H), 7.84 (s, 1H), 7.42 (d, J=8.3 Hz, 2H), 7.37 (d, J=8.3 Hz, 2H), 7.31-7.22 (m, 2H), 7.07 (d, J=7.8 Hz, 1H), 7.02-6.95 (m, 1H), 4.73-4.50 (m, 3H), 3.97 (t, J=6.6 Hz, 2H), 3.65-3.57 (m, 4H), 3.40 (t, J=6.4 Hz, 2H), 2.77-2.66 (m, 4H), 1.68 (s, 2H), 1.48 (dd, J=14.1, 7.5 Hz, 8H).

### Example 51: Synthesis of Compound UB-051

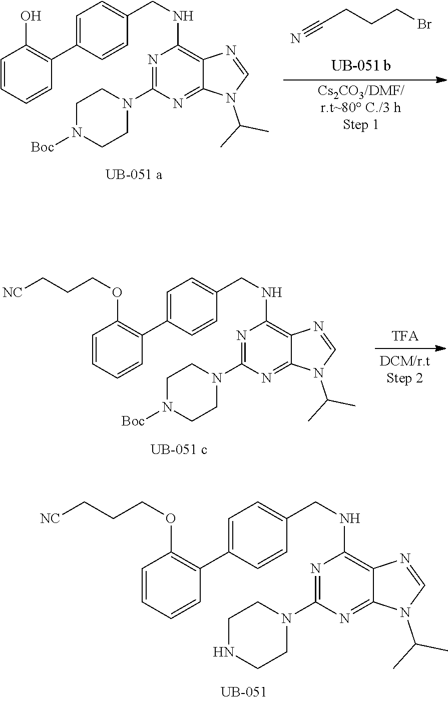 [SMILES: CC(C)n1cnc2c(NCc3ccc(-c4ccccc4O)cc3)nc(N3CCN(C(=O)OC(C)(C)C)CC3)nc21 ; N#CCCCBr ; CC(C)n1cnc2c(NCc3ccc(-c4ccccc4OCCCC#N)cc3)nc(N3CCN(C(=O)OC(C)(C)C)CC3)nc21 ; CC(C)n1cnc2c(NCc3ccc(-c4ccccc4OCCCC#N)cc3)nc(N3CCNCC3)nc21]

### Step 1: UB-051c

### Tert-butyl 4-(6-(((2′-(3-cyanopropoxy)-[1,1′-biphenyl]-4-yl)methyl)amino)-9-isopropyl-9H-purin-2-yl)piperazin-1-carboxylate

Under N₂, a solution of UB-051a (100 mg, 0.185 mmol), Cs2CO₃ (120 mg, 0.925 mmol) and DMF (5 mL) was added to a reaction flask. The reaction solution turned yellow after stirring at room temperature. Then UB-051b (55 mg, 0.925 mmol) was added and the reaction solution was heated at 80° C. for 3 hours. The reaction solution was then cooled to room temperature and filtered. The solvent was evaporated and the obtained residue was purified by silica gel column chromatography [eluent: PE/EA=0-80%] to obtain a colorless oil compound (UB-051c, 100 mg, yield 88.6%). LCMS [M+1]⁺=611.3

### Step 2: UB-051

### 4-((4′-(((9-isopropyl-2-(piperazin-1-yl)-9H-purin-6-yl)amino)methyl)-[1,1′-biphenyl]-2-yl)oxy)butyronitrile

UB-051c (100 mg, 0.16 mmol), 6 mL of DCM and trifluoroacetic acid (2 mL) were added to a reaction flask. The mixture was stirred at room temperature for 1 hour. The reaction mixture was quenched with NaHCO₃ (4 mL) and extracted with DCM (20 mL*3). The organic phase was separated, washed with brine (20 mL*3) and dried over Na₂SO₄, then concentrated and purified by flash chromatography, using DCM/(DCM:MeOH=10:1)═0-100% as eluent, to obtain a white solid compound (UB-051, 11.4 mg, yield 14%) LCMS [M+1]⁺=511.2. ¹H NMR (400 MHz, DMSO-d₆) δ 7.99 (s, 1H), 7.85 (s, 1H), 7.44-7.37 (m, 4H), 7.34-7.24 (m, 2H), 7.10 (d, J=8.2 Hz, 1H), 7.02 (t, J=7.5 Hz, 1H), 4.78-4.43 (m, 3H), 4.03 (t, J=6.0 Hz, 2H), 3.62 (s, 4H), 2.73 (s, 4H), 2.53 (d, J=5.2 Hz, 2H), 1.95 (p, J=6.6 Hz, 2H), 1.47 (d, J=6.8 Hz, 6H).

### Example 52: Synthesis of Compound UB-052

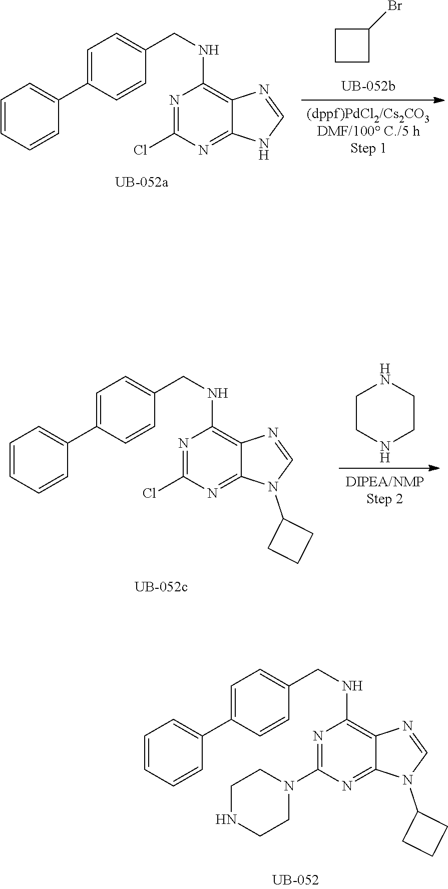 [SMILES: Clc1nc(NCc2ccc(-c3ccccc3)cc2)c2nc[nH]c2n1 ; BrC1CCC1 ; Clc1nc(NCc2ccc(-c3ccccc3)cc2)c2ncn(C3CCC3)c2n1 ; C1CNCCN1 ; c1ccc(-c2ccc(CNc3nc(N4CCNCC4)nc4c3ncn4C3CCC3)cc2)cc1]

### Step 1: UB-052c

### N-([1,1′-biphenyl]-4-ylmethyl)-2-chloro-9-cyclobutyl-9H-purin-6-amine

To a 100 mL round-bottom flask were added in sequence compound UB-052a (100 mg, 0.298 mmol), UB-052b (60 mg, 0.446 mmol), (dppf)PdCl₂ (22 mg, 0.029 mmol), Cs2CO₃ (386 mg, 1.19 mmol), and DMF (5 mL). The resulting mixture was replaced with argon three times and then heated to 100° C. and reacted for 5 hours. After the reaction was completed, the mixture was concentrated under reduced pressure. The residue was separated by column chromatography (eluent: DCM:PE:EA=100/0-80/20) to obtain a light yellow solid compound UB-052c (80 mg, yield 46%). LCMS [M+1]⁺=390.1.

### Step 2: UB-052

### N-([1,1′-biphenyl]-4-ylmethyl)-9-cyclobutyl-2-(piperazin-1-yl)-9H-purin-6-amine

UB-052c (60 mg, 0.15 mmol), UB-052d (66 mg, 0.77 mmol), DIPEA (158 mg, 1.23 mmol), and NMP (3 mL) were added to a 10 mL microwave tube. The resulting mixture was reacted at 180° C. under microwave for 3 hours. After the reaction finished, 30 mL of water was added. The solid precipitated and was filtered. The filter cake was separated by column chromatography (eluent: DCM/(10% MeOH in DCM)═100/0-30/70) to obtain a white solid compound UB-052 (30 mg, yield 45%). LCMS [M+H]⁺=440.2. ¹H NMR (400 MHz, DMSO-d₆) δ 8.01 (s, 1H), 7.94 (s, 1H), 7.66-7.60 (m, 2H), 7.58 (d, J=8.2 Hz, 2H), 7.49-7.39 (m, 4H), 7.33 (t, J=7.3 Hz, 1H), 4.92-4.75 (m, 1H), 4.63 (s, 2H), 3.72-3.49 (m, 5H), 2.71 (dd, J=14.2, 9.6 Hz, 4H), 2.59 (ddd, J=19.3, 9.7, 2.5 Hz, 2H), 2.42-2.27 (m, 2H), 1.92-1.73 (m, 2H).

### Example 53: Synthesis of Compound UB-053

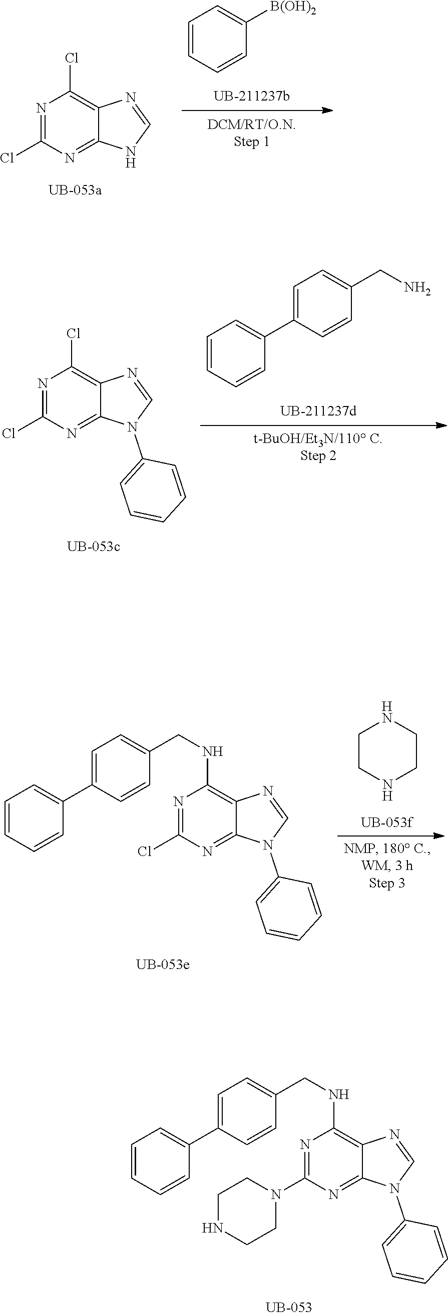

### Step 1: UB-053c

### Tert-butyl 2,6-Dichloro-9-phenyl-9H-purine

UB-053b (645.01 mg, 5.29 mmol), Cu(OAc)₂ (960.82 mg, 5.29 mmol), and TEA (804.46 mg, 7.95 mmol) were added to a solution of UB-053a (519.40 mg, 2.65 mmol) in dichloromethane (20 mL), and the mixture was stirred at room temperature for 1 hour. The reaction mixture was concentrated and purified by chromatography (dichloromethane) to obtain a white solid (UB-053c, 63.1 mg, yield: 7.5%) LCMS [M+1]⁺=265.0; 267.0.

### Step 2: UB-053e

### N-([1,1′-biphenyl]-4-ylmethyl)-2-chloro-9-phenyl-9-hydrogen-purine-6-amine

UB-053d (63.07 mg, 0.24 mmol) and TEA (96.33 mg, 0.95 mmol) were added to a solution of UB-053c (43.55 mg, 0.24 mmol) in tert-butanol (4 mL), and the mixture was stirred at 110° C. overnight. After the reaction was completed, the mixture was filtered and the filter cake was washed with ethanol:water (10:1) (20 mL), and the filter cake was drained to obtain a white solid (UB-053e, 67.6 mg, yield: 69%). LCMS [M+1]⁺=412.1.

### Step 3: UB-053

### N-([1,1′-biphenyl]-4-ylmethyl)-9-phenyl-2-(piperazin-1-yl)-9H-purin-6-amine

UB-053e (41.2 mg, 0.10 mmol), UB-053f (41.3 mg, 0.48 mmol), DIPEA (98.2 mg, 0.76 mmol) and NMP (3 mL) were added to a microwave tube and the mixture was reacted under microwave at 180° C. for 3 h. The reaction mixture was diluted with water (200 mL) and extracted with dichloromethane (3×70 mL). The combined organic phase was washed with brine, dried over anhydrous sodium sulfate and concentrated under reduced pressure. The crude product was purified by silica gel chromatography (dichloromethane: dichloromethane/methanol: 10/1=10:90) to give a white solid (UB-053, 34.0 mg, yield: 51.15%). LCMS [M+1]⁺=462.2. ¹H NMR (400 MHz, DMSO-d₆) δ 9.07 (s, 2H), 8.50 (s, 1H), 8.43 (s, 1H), 7.88 (d, J=7.8 Hz, 2H), 7.65-7.60 (m, 4H), 7.57 (t, J=7.9 Hz, 2H), 7.50-7.39 (m, 5H), 7.35 (t, J=7.3 Hz, 1H), 4.69 (s, 2H), 3.89 (d, J=4.7 Hz, 4H), 3.08 (s, 4H).

### Example 54: Synthesis of Compound UB-054

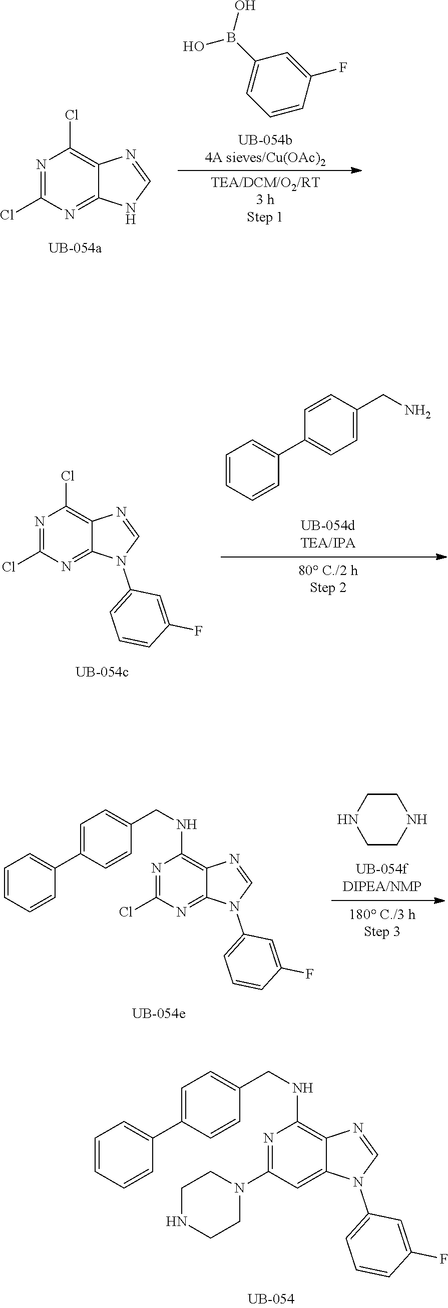

### Step 1: UB-054c

### 2,6-Dichloro-9-(3-fluorophenyl)-9H-purine

Compound UB-054b (740 mg, 5.29 mmol) and compound UB-054a (500 mg, 2.64 mmol) were dissolved in (10 mL) dichloromethane, and copper acetate (961 mg, 5.29 mmol) and triethylamine (804 mg, 7.96 mmol) were added. The reaction was carried out at room temperature for 3 hours. After the reaction was completed, the reaction solution was concentrated and purified by silica gel column (dichloromethane) to obtain the compound (UB-054c, 200 mg, 26% yield) as a yellow solid. LCMS [M+1]⁺=283.0&284.9.

### Step 2: UB-054e

### N-([1,1′-biphenyl]-4-ylmethyl)-2-chloro-9-(3-fluorophenyl)-9H-purin-6-amine

Compound UB-054c (200 mg, 0.71 mmol), UB-054d (120 mg, 0.68 mmol) and triethylamine (206 mg, 2.04 mmol) were dissolved in isopropanol (3 mL). The reaction was stirred at 80° C. for 2 hours. After the reaction was completed, the reaction solution was cooled to room temperature. The reaction solution was filtered to obtain the desired compound UB-054e (75 mg, 53% yield) as a white solid. LCMS [M+1]⁺=430.1.

### Step 3: UB-054

### N-([1,1′-biphenyl]-4-ylmethyl)-9-(3-fluorophenyl)-2-(piperazin-1-yl)-9H-purin-6-amine

Compound UB-054f (40 mg, 0.47 mmol), UB-054e (40 mg, 0.09 mmol) and DIPEA (96 mg, 0.74 mmol) were dissolved in NMP (3 mL). The reaction was stirred under microwave at 180° C. for 3 hours. After the reaction was completed, the reaction solution was cooled to room temperature. Water was added until solid precipitated from the reaction solution and the solid was filtered. The filter residue was dissolved and purified by silica gel column (DCM/MeOH=30%) and lyophilized to give the desired compound UB-0450 (16 mg, 20% yield) as a white solid. LCMS [M+1]⁺=480.2. ¹H NMR (400 MHz, DMSO-d₆) δ 8.34 (s, 1H), 8.25 (s, 1H), 7.92 (dt, J=10.8, 2.2 Hz, 1H), 7.88-7.83 (m, 1H), 7.65-7.56 (m, 5H), 7.48-7.41 (m, 4H), 7.34 (t, J=7.3 Hz, 1H), 7.22 (td, J=8.5, 2.2 Hz, 1H), 4.67 (s, 2H), 3.70-3.57 (m, 4H), 2.81-2.66 (m, 4H), 1.29 (d, J=49.5 Hz, 3H).

### Example 55: Synthesis of Compound UB-055

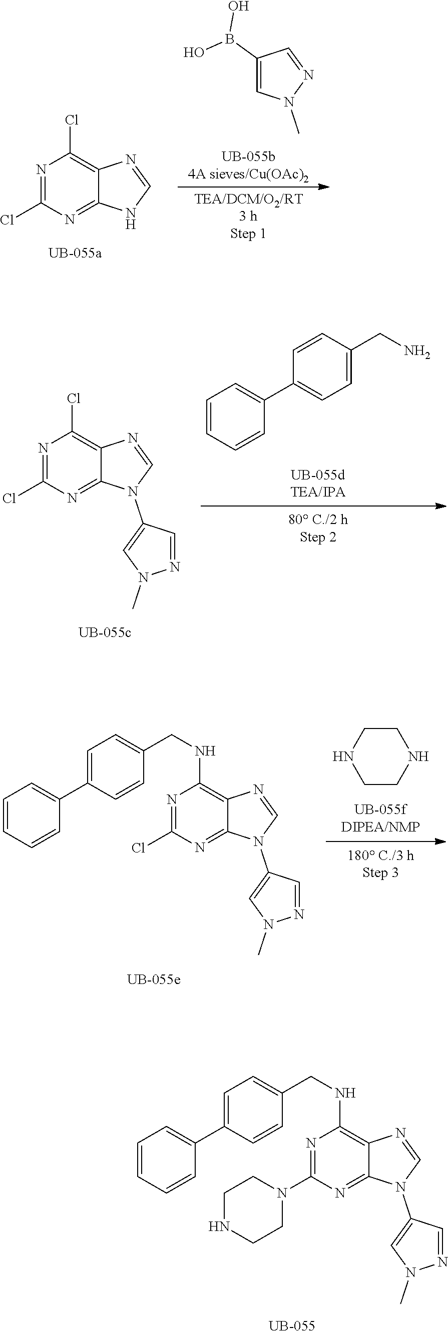

### Step 1: UB-055c

### 2,6-Dichloro-9-(1-methyl-1H-pyrazol-4-yl)-9H-purine

Compound UB-055b (666 mg, 5.29 mmol) and compound UB-055a (500 mg, 2.64 mmol) were dissolved in (10 mL) dichloromethane, and copper acetate (961 mg, 5.29 mmol) and triethylamine (804 mg, 7.96 mmol) were added. The reaction was carried out at room temperature for 3 hours. After the reaction was completed, the reaction solution was concentrated and purified by silica gel column (dichloromethane) to obtain the compound (UB-055c, 120 mg, 16.9% yield) as a yellow solid. LCMS [M+1]⁺=269.1.

### Step 2: UB-055e

### N-([1,1′-biphenyl]-4-ylmethyl)-2-chloro-9-(1-methyl-1H-pyrazol-4-yl)-9H-purin-6-amine

Compound UB-055c (120 mg, 0.45 mmol), UB-055d (80 mg, 0.43 mmol) and triethylamine (130 mg, 1.29 mmol) were dissolved in isopropanol (2 mL). The reaction was stirred at 80° C. for 2 hours. After the reaction was completed, the reaction solution was cooled to room temperature. The reaction solution was filtered to obtain the desired compound

UB-055e (95 mg, 53% yield) as a white solid. LCMS [M+1]⁺=416.2.

### Step 3: UB-055

### N-([1,1′-biphenyl]-4-ylmethyl)-9-(1-methyl-1H-pyrazol-4-yl)-2-(piperazin-1-yl)-9H-purin-6-amine

Compound UB-055f (99 mg, 1.15 mmol), UB-055e (95 mg, 0.23 mmol) and DIPEA (238 mg, 1.84 mmol) were dissolved in NMP (3 mL). The reaction was stirred under microwave at 180° C. for 3 hours. After the reaction was completed, the reaction solution was cooled to room temperature. Water was added until solid precipitated from the reaction solution and the solid was filtered. The filter residue was dissolved and purified by silica gel column (DCM/MeOH=60%) and lyophilized to give the desired compound UB-055 (64 mg, 59% yield) as a white solid. LCMS [M+1]⁺=466.2. ¹H NMR (400 MHz, DMSO-d₆) δ 8.29 (s, 1H), 8.29 (s, 1H), 8.28-8.12 (m, 2H), 8.15 (d, J=4.4 Hz, 1H), 7.98 (s, 1H), 7.98 (s, 1H), 7.68-7.55 (m, 4H), 7.66-7.58 (m, 4H), 7.48-7.42 (m, 4H), 7.52-7.39 (m, 4H), 7.34 (t, J=7.3 Hz, 1H), 7.34 (t, J=7.3 Hz, 1H), 4.66 (s, 2H), 4.66 (s, 2H), 3.92 (s, 3H), 3.92 (s, 3H), 3.69-3.61 (m, 4H), 3.75-3.58 (m, 4H), 2.74 (s, 4H), 2.74 (s, 4H).

### Examples 59-63: Synthesis of Compounds UB-056 to UB-063

The synthesis of compounds UB-059, and UB-060 are similar to the synthesis of UB-032.

The synthesis of compound UB-061 is similar to the synthesis of UB-041.

The synthesis of compounds UB-062, and UB-063 are similar to the synthesis of UB-043.

TABLE A1

| Structure & code | | |
|---|---|---|
| name | Name & properties | LCMS [M + 1]⁺ & ¹H-NMR |
| 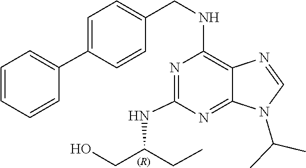 | (R)-2-((6-(([1,1′-biphenyl]- 4-ylmethyl)amino)- 9-isopropyl-9H-purin-2- yl)amino)butan-1-ol Yellow solid compound (Yield 31.2%) | MS[M + 1]⁺ = 431.6; ¹H NMR (400 MHz, CDCl₃) δ 7.66-7.58 (m, 4H), 7.57 (s, 1H), 7.53-7.44 (m, 4H), 7.38 (t, J = 7.3 Hz, 1H), 6.77 (s, 1H), 5.00 (d, J = 6.0 Hz, 1H), 4.83 (s, 2H), 4.61 (dt, J = 13.5, 6.8 Hz, 1H), 4.01- 3.91 (m, 1H), 3.85 (dd, J = 10.7, 2.3 Hz, 1H), 3.67 (dd, J = 10.5, 7.6 Hz, 1H), 1.73-1.57 (m, 2H), 1.53 (d, J = 6.7 Hz, 6H), 1.05 (t, J = 7.4 Hz, 3H). |
| UB-007 | | |
| 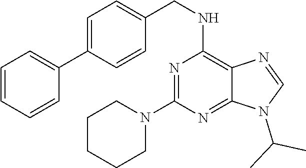 [SMILES: CC(C)n1cnc2c(NCc3ccc(-c4ccccc4)cc3)nc(N3CCCCC3)nc21] | N-([1,1′-biphenyl]-4-yl methyl)-9-isopropyl-2- (Piperidin-1-yl)-9H-purin- 6-amine Yellow solid (Yield 14.8%) | MS[M + 1]⁺ = 427.5. |
| UB-008 | | |
| 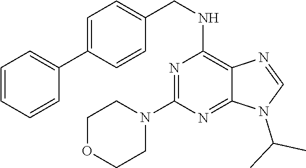 [SMILES: CC(C)n1cnc2c(NCc3ccc(-c4ccccc4)cc3)nc(N3CCOCC3)nc21] | N-([1,1′-biphenyl]-4-yl methyl)-9-isopropyl-2- morpholino-9H-purin- 6-amine Yellow solid compound (Yield 17%) | MS[M + 1]⁺ = 429.6; ¹H NMR (400 MHz, CDCl₃) δ 7.68 (s, 1H), 7.59 (dd, J = 15.8, 7.8 Hz, 4H), 7.51-7.43 (m, 4H), 7.38 (t, J = 7.3 Hz, 1H), 6.64 (s, 1H), 4.85 (s, 2H), 4.75- 4.60 (m, 1H), 3.80 (d, J = 3.4 Hz, 8H), 1.56 (d, J = 6.7 Hz, 6H). |
| UB-009 | | |
| 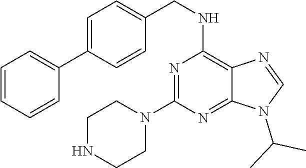 [SMILES: CC(C)n1cnc2c(NCc3ccc(-c4ccccc4)cc3)nc(N3CCNCC3)nc21] | N-([1,1′-biphenyl]-4-yl methyl)-9-isopropyl-2- (piperazin-1-yl)-9H-purin- 6-amine Yellow solid compound (Yield 37.5%) | MS[M + 1]⁺ = 428.6; ¹H NMR (400 MHz, CDCl₃) δ 7.80 (s, 1H), 7.57 (dd, J = 12.3, 7.9 Hz, 4H), 7.45 (dd, J = 12.5, 7.7 Hz, 4H), 7.36 (s, 1H), 4.79 (s, 2H), 4.66 (dt, J = 12.9, 6.3 Hz, 2H), 4.11 (s, 4H), 3.22 (s, 4H), 1.55 (d, J = 6.7 Hz, 6H). |
| UB-010 | | |
| 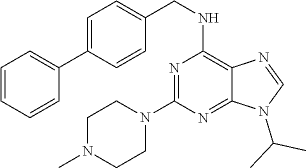 [SMILES: CC(C)n1cnc2c(NCc3ccc(-c4ccccc4)cc3)nc(N3CCN(C)CC3)nc21] | N-([1,l′-biphenyl]-4-yl methyl)-9-isopropyl-2- (4-methylpiperazin-1-yl)- 9H-purin-6-amine Yellow solid compound (Yield 42%) | MS[M + 1]⁺ = 442.7; ¹H NMR (400 MHz, CDCl₃) δ 7.59 (dd, J = 12.8, 7.9 Hz, 5H), 7.50-7.44 (m, 4H), 7.37 (t, J = 7.3 Hz, 1H), 6.43 (s, 1H), 4.85 (s, 2H), 4.73-4.63 (m, 1H), 4.00 (s, 4H), 2.67 (s, 4H), 2.49 (s, 3H), 1.56 (d, J = 6.8 Hz, 6H). |
| UB-011 | | |
| 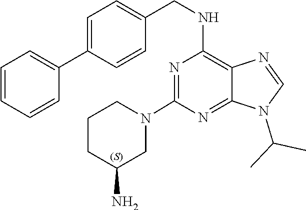 | (S)-N-([1,1′-biphenyl]- 4-ylmethyl)-2-(3-amino piperidin-1-yl)-9-isopropyl- 9H-purin-6-amine Yellow solid compound (Yield 66%) | MS[M + 1]⁺ = 442.7; ¹H NMR (500 MHz, DMSO-d₆) δ 8.17 (s, 2H), 7.81 (s, 1H), 7.61 (dd, J = 15.5, 7.7 Hz, 4H), 7.44 (t, J = 7.8 Hz, 4H), 7.34 (t, J = 7.3 Hz, 1H), 6.17 (s, 1H), 4.63 (s, 2H), 4.57-4.44 (m, 1H), 3.61 (d, J = 7.2 Hz, 1H), 2.91 (t, J = 11.1 Hz, 1H), 1.95 (t, J = 15.9 Hz, 4H), 1.45 (t, J = 12.4 Hz, 7H), 1.32-1.07 (m, 2H). |
| UB-012 | | |
| 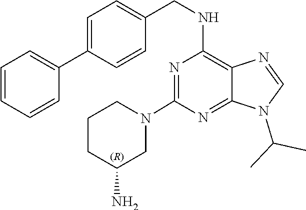 | (R)-N-([1,1′-biphenyl]- 4-ylmethyl)-2-(3-amino piperidin-1-yl)-9-isopropyl- 9H-purin-6-amine Yellow solid compound (Yield 38%) | MS[M + 1]⁺ = 442.7; ¹H NMR (500 MHz, DMSO-d₆) δ 8.09 (s, 1H), 7.89 (s, 1H), 7.61 (dd, J = 18.2, 7.8 Hz, 4H), 7.50 (d, J = 7.7 Hz, 2H), 7.44 (t, J = 7.7 Hz, 2H), 7.34 (t, J = 7.3 Hz, 1H), 4.75-4.56 (m, 3H), 4.30 (d, J = 12.9 Hz, 1H), 3.54-3.21 (m, 2H), 3.09 (dd, J = 13.9, 6.2 Hz, 2H), 3.00 (t, J = 10.9 Hz, 1H), 2.51 (s, 1H), 2.01 (d, J = 10.0 Hz, 1H), 1.73 (dd, J = 9.2, 4.0 Hz, 1H), 1.63-1.52 (m, 1H), 1.47 (t, J = 10.0 Hz, 7H). |
| UB-013 | | |
| 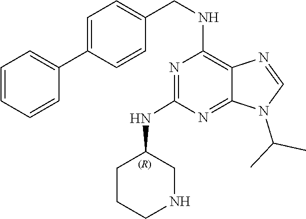 | (R)-N6-([1,1′-biphenyl]- 4-ylmethyl)-9-isopropyl- N2-(Piperidin-3-yl)- 9H-purin-2,6-diamine Yellow solid compound (Yield 17.6%) | MS[M + 1]⁺ = 442.5; ¹H NMR (400 MHz, DMSO-d₆) δ 8.05 (s, 1H), 7.90 (s, 1H), 7.63 (dd, J = 14.2, 8.0 Hz, 4H), 7.48 (dd, J = 16.7, 8.5 Hz, 4H), 7.37 (t, J = 7.3 Hz, 1H), 4.80- 4.47 (m, 4H), 4.31 (d, J = 12.9 Hz, 1H), 3.03 (d, J = 7.5 Hz, 4H), 2.02-1.92 (m, 1H), 1.79- 1.69 (m, 1H), 1.51 (d, J = 6.8 Hz, 9H). |
| UB-014 | | |
| 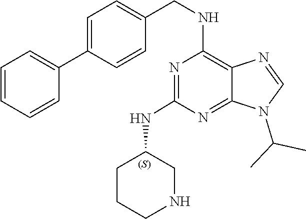 | (S)-N6-([1,1′-biphenyl]- 4-ylmethyl)-9-isopropyl- N2-(Piperidin-3-yl)- 9H-purin-2,6-diamine Yellow solid compound (Yield 22.6%) | MS[M + 1]⁺ = 442.5. |
| UB-015 | | |
| 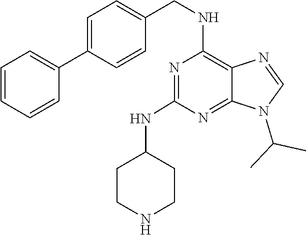 [SMILES: CC(C)n1cnc2c(NCc3ccc(-c4ccccc4)cc3)nc(NC3CCNCC3)nc21] | N6-([1,1′-biphenyl]-4-yl- methyl)-9-isopropyl-N2- (Piperidin-4-yl)-9H- purin-2,6-diamine Yellow solid compound (Yield 28.4%) | MS[M + 1]⁺ = 442.3; ¹H NMR (400 MHz, DMSO-d₆) δ 7.80 (s, 1H), 7.59 (dd, J = 17.4, 7.8 Hz, 4H), 7.47-7.40 (m, 4H), 7.33 (t, J = 7.3 Hz, 1H), 6.17 (d, J = 7.2 Hz, 1H), 4.64 (s, 2H), 4.52 (dt, J = 13.2, 6.5 Hz, 1H), 3.72 (s, 1H), 2.95 (d, J = 11.3 Hz, 2H), 2.56 (d, J = 11.2 Hz, 1H), 1.86 (s, 2H), 1.78 (s, 2H), 1.46 (d, J = 6.7 Hz, 6H), 1.32 (dd, J = 16.4, 8.8 Hz, 2H), 1.24 (d, J = 6.1 Hz, 1H). |
| UB-016 | | |
| 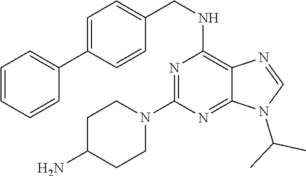 [SMILES: CC(C)n1cnc2c(NCc3ccc(-c4ccccc4)cc3)nc(N3CCC(N)CC3)nc21] | N-([1,1′-biphenyl]-4-yl methyl)-2-(4-amino- piperidin-1-yl)-9-isopropyl- 9H-purin-6-amine Yellow solid compound (Yield 66%) | MS[M + 1]⁺ = 442.6; ¹H NMR (500 MHz, DMSO-d₆) δ 8.09 (s, 1H), 7.87 (s, 1H), 7.78 (s, 2H), 7.65-7.60 (m, 2H), 7.58 (d, J = 8.2 Hz, 2H), 7.48-7.42 (m, 4H), 7.34 (t, J = 7.4 Hz, 1H), 4.68-4.54 (m, 4H), 3.26-3.16 (m, 1H), 2.86 (t, J = 11.7 Hz, 2H), 2.54 (s, 1H), 1.88 (d, J = 10.5 Hz, 2H), 1.47 (d, J = 6.8 Hz, 6H), 1.37 (dd, J = 19.5, 10.2 Hz, 2H). |
| UB-017 | | |
| 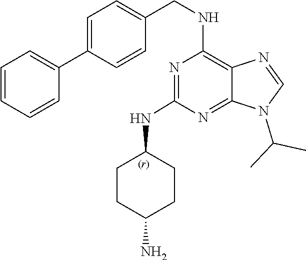 | N6-([1,1′-biphenyl]-4-yl- methyl)-N2-((1r,4r)-4- aminocyclohexyl)-9- isopropyl-9H-purin-2,6- diamine Yellow solid compound (Yield 37%) | MS[M + 1]⁺ = 456.3; ¹H NMR (500 MHz, DMSO-d₆) δ 8.08 (s, 1H), 7.89 (s, 2H), 7.62 (d, J = 7.3 Hz, 2H), 7.59 (d, J = 8.2 Hz, 2H), 7.48 (d, J = 7.9 Hz, 2H), 7.44 (t, J = 7.7 Hz, 2H), 7.34 (t, J = 7.3 Hz, 1H), 4.79-4.49 (m, 4H), 4.27 (d, J = 13.1 Hz, 1H), 3.15-2.97 (m, 3H), 2.54 (s, 1H), 2.05-1.94 (m, 1H), 1.73 (dd, J = 8.9, 4.4 Hz, 1H), 1.54 (d, J = 8.7 Hz, 1H), 1.48 (d, J = 6.8 Hz, 8H). |
| UB-018 | | |
| 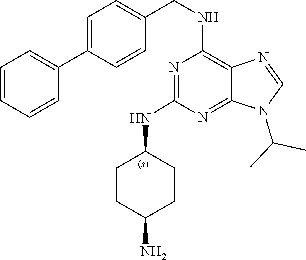 | N6-([1,1′-biphenyl]-4-yl- methyl)-N2-((1s,4s)-4- aminocyclohexyl)-9- isopropyl-9H-purin-2,6- diamine Yellow solid compound (Yield 11.4%) | MS[M + 1]⁺ = 456.6. |
| UB-019 | | |
| 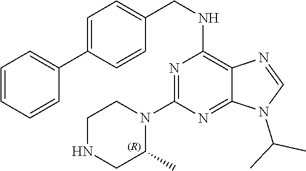 | (R)-N-([1,1′-biphenyl]- 4-ylmethyl)-9-isopropyl- 2-(2-methylpiperazin-1- yl)-9H-purin-6-amine White solid compound (Yield 47.5%) | MS[M + 1]⁺ = 442.3; ¹H NMR (400 MHz, DMSO-d₆) δ 7.99 (s, 1H), 7.84 (s, 1H), 7.64 7.59 (m, 2H), 7.58 (d, J = 8.2 Hz, 2H), 7.47- 7.40 (m, 4H), 7.33 (t, J = 7.3 Hz, 1H), 4.70 (dd, J = 15.0, 11.6 Hz, 2H), 4.56 (dt, J = 13.4, 6.7 Hz, 2H), 4.38-4.29 (m, 1H), 3.00-2.83 (m, 2H), 2.79 (s, 2H), 1.47 (dd, J = 6.7, 1.0 Hz, 6H), 1.08 (d, J = 6.5 Hz, 3H). |
| UB-020 | | |
| 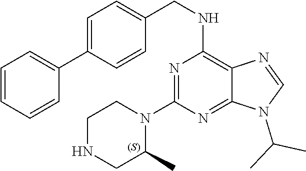 | (S)-N-([1,1′-biphenyl]- 4-ylmethyl)-9-isopropyl- 2-(2-methylpiperazin-1- yl)-9H-purin-6-amine White solid compound (Yield 10%) | MS [M + 1]⁺ = 442.3;. 1H NMR (400 MHz, DMSO-d₆) δ 8.11 (s, 1H), 7.88 (s, 1H), 7.60 (dd, J = 13.8, 8.0 Hz, 4H), 7.47-7.42 (m, 4H), 7.34 (t, J = 7.3 Hz, 1H), 4.87 (s, 1H), 4.56 (ddd, J = 33.3, 26.3, 16.3 Hz, 4H), 3.15 (d, J = 12.0 Hz, 1H), 3.09-2.90 (m, 3H), 2.74 (t, J = 22.8 Hz, 1H), 1.47 (d, J = 6.7 Hz, 6H), 1.24 (d, J = 6.0 Hz, 1H), 1.12 (d, J = 6.5 Hz, 3H). |
| UB-021 | | |
| 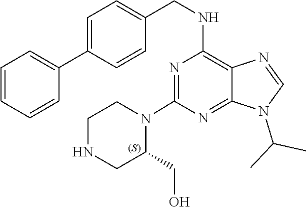 | (S)-(1-(6-(([1,1′-biphenyl]- 4-ylmethyl)amino)- 9-isopropyl-9H-purin-2- yl)piperazin-2-yl) methanol White solid compound (Yield 29%) | MS [M + 1]⁺ = 458.3; ¹H NMR (400 MHz, DMSO-d₆) δ 8.00 (s, 1H), 7.84 (s, 1H), 7.65- 7.60 (m, 2H), 7.58 (d, J = 8.2 Hz, 2H), 7.48- 7.40 (m, 4H), 7.33 (t, J = 7.3 Hz, 1H), 4.67- 4.51 (m, 4H), 4.43 (d, J = 12.9 Hz, 1H), 3.79 (t, J = 9.3 Hz, 1H), 3.42 (dd, J = 9.8, 4.7 Hz, 2H), 3.23 (d, J = 12.3 Hz, 2H), 3.00-2.87 (m, 2H), 2.68 (dd, J = 12.3, 4.0 Hz, 1H), 2.64- 2.54 (m, 1H), 1.46 (d, J = 6.2 Hz, 6H). |
| UB-022 | | |
| 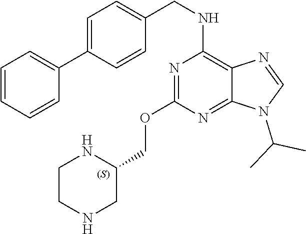 | (S)-N-([1,l′-biphenyl]- 4-ylmethyl)-9-isopropyl- 2-(piperazin-2-ylmethoxy)- 9H-purin-6-amine Light yellow solid compound (Yield 60%) | 458.3; ¹H NMR (400 MHz, DMSO-d₆) δ 7.94 (s, 1H), 7.82 (s, 1H), 7.65-7.60 (m, 2H), 7.58 (d, J = 8.2 Hz, 2H), 7.48-7.41 (m, 4H), 7.33 (t, J = 7.3 Hz, 1H), 4.62 (s, 2H), 4.55 (m, 3H), 4.37 (d, J = 12.5 Hz, 1H), 3.80 (t, J = 9.4 Hz, 1H), 3.42-3.35 (m, 2H), 3.16 (d, J = 12.0 Hz, 1H), 2.86 (t, J = 12.8 Hz, 2H), 2.58 (dd, J = 12.1, 3.8 Hz, 1H), 1.46 (dd, J = 6.7, 0.9 Hz, 6H). |
| UB-033 | | |
| 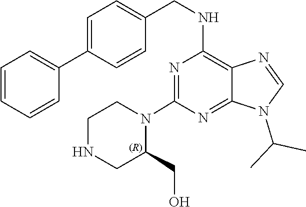 | (R)-(1-(6-(([1,1′-biphenyl]- 4-ylmethyl)amino)- 9-isopropyl-9H-purin-2- yl)piperazin-2-yl) methanol Light yellow solid compound (Yield 15.5%) | MS[M + 1]⁺ = 458.3; ¹H NMR (400 MHz, DMSO-d₆) δ 8.09 (s, 1H), 7.87 (s, 1H), 7.65- 7.60 (m, 2H), 7.58 (d, J = 8.2 Hz, 2H), 7.48 7.41 (m, 4H), 7.34 (t, J = 7.3 Hz, 1H), 4.70 (s, 1H), 4.67-4.58 (m, 2H), 4.54 (dd, J = 13.3, 5.7 Hz, 2H), 3.78 (t, J = 9.2 Hz, 1H), 3.49 (dd, J = 10.1, 4.8 Hz, 1H), 3.37 (s, 1H), 3.07 (dd, J = 24.9, 12.1 Hz, 2H), 2.84 (dd, J = 12.3, 3.7 Hz, 1H), 2.71 (dd, J = 23.4, 11.4 Hz, 1H), 1.50-1.44 (m, 6H). |
| UB-023 | | |
| 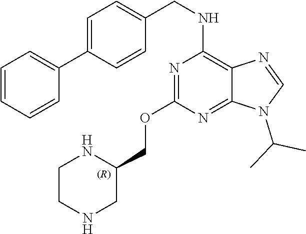 | (R)-N-([1,1′-biphenyl]- 4-ylmethyl)-9-isopropyl- 2-(piperazin-2-ylmethoxy)- 9H-purin-6-amine Light yellow solid compound (Yield 49%) | MS[M + 1]⁺ = 458.3; ¹H NMR (400 MHz, DMSO-d₆) δ 8.39 (s, 1H), 8.05 (s, 1H), 7.61 (dd, J = 11.1, 7.8 Hz, 4H), 7.43 (dd, J = 11.9, 5.2 Hz, 4H), 7.35 (d, J = 7.3 Hz, 1H), 4.80- 4.50 (m, 3H), 4.06 (d, J = 5.6 Hz, 2H), 2.86 (d, J = 9.2 Hz, 2H), 2.77 (d, J = 10.6 Hz, 1H), 2.70 (d, J = 11.1 Hz, 1H), 2.61-2.55 (m, 1H), 2.32 (d, J = 10.7 Hz, 1H), 1.49 (d, J = 6.7 Hz, 6H). |
| UB-034 | | |
| 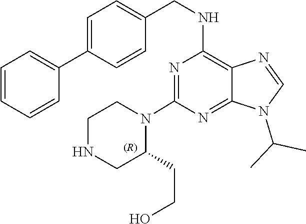 | (R)-2-(1-(6-(([1,1′-biphenyl]- 4-ylmethyl)amino)- 9-isopropyl-9H-purin- 2-yl)piperazin-2-yl) ethan-1-ol White solid compound (Yield 32%) | MS[M + 1]⁺ = 472.3; ¹H NMR (400 MHz, DMSO-d₆) δ 7.97 (s, 1H), 7.83 (s, 1H), 7.62 (dd, J = 5.2, 3.3 Hz, 2H), 7.58 (d, J = 8.3 Hz, 2H), 7.44 (dd, J = 8.0, 6.8 Hz, 4H), 7.37- 7.29 (m, 1H), 4.65 (s, 3H), 4.53 (m, 1H), 4.44 (d, J = 11.2 Hz, 1H), 3.36 (t, J = 6.9 Hz, 2H), 2.91-2.77 (m, 3H), 2.69-2.60 (m, 1H), 1.84 (d, J = 6.6 Hz, 2H), 1.46 (dd, J = 6.7, 3.4 Hz, 6H). |
| UB-024 | | |
| 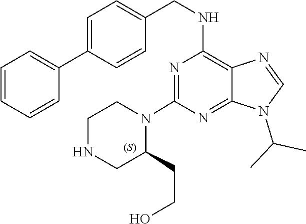 | (S)-2-(1-(6-(([1,1′-biphenyl]- 4-ylmethyl)amino)- 9-isopropyl-9H-purin- 2-yl)piperazin-2-yl) ethan-1-ol White solid compound (Yield 33.8%) | MS[M + 1]⁺ = 472.2; ¹H NMR (400 MHz, DMSO-d₆) δ 8.00 (s, 1H), 7.84 (s, 1H), 7.64- 7.60 (m, 2H), 7.58 (d, J = 8.2 Hz, 2H), 7.47- 7.41 (m, 4H), 7.34 (d, J = 7.4 Hz, 1H), 4.66 (d, J = 30.1 Hz, 3H), 4.54 (dd, J = 13.5, 6.8 Hz, 1H), 4.50-4.44 (m, 1H), 3.37 (t, J = 6.7 Hz, 3H), 2.91 (t, J = 10.9 Hz, 3H), 2.75- 2.64 (m, 1H), 1.93-1.80 (m, 2H), 1.46 (dd, J = 6.7, 3.2 Hz, 6H). |
| UB-025 | | |
| 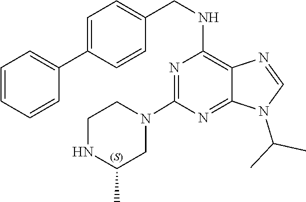 | (S)-N-([1,1′-biphenyl]- 4-ylmethyl)-9-isopropyl- 2-(3-methylpiperazin-1- yl)-9H-purin-6-amine White solid compound (Yield 30%) | MS[M + 1]⁺ = 442; ¹H NMR (400 MHz, CDCl₃) δ 7.99 (s, 1H), 7.84 (s, 1H), 7.65- 7.54 (m, 4H), 7.44 (dt, J = 7.8, 3.6 Hz, 4H), 7.38-7.29 (m, 1H), 4.57 (td, J = 13.5, 13.0, 6.2 Hz, 3H), 4.44 (dd, J = 12.7, 3.0 Hz, 2H), 2.86 (d, J = 11.1 Hz, 1H), 2.68 (td, J = 12.0, 2.7 Hz, 1H), 2.57 (td, J = 11.4, 4.7 Hz, 3H), 2.31 (dd, J = 12.5, 10.3 Hz, 1H), 1.46 (d, J = 6.7 Hz, 6H), 0.98 (d, J = 6.2 Hz, 3H). |
| UB-026 | | |
| 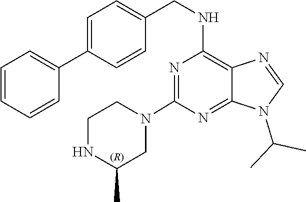 | (R)-N-([1,1′-biphenyl]- 4-ylmethyl)-9-isopropyl- 2-(3-methylpiperazin-1- yl)-9H-purin-6-amine White solid compound (Yield 45%) | MS[M + 1]⁺ = 442.3; ¹H NMR (400 MHz, DMSO-d₆) δ 7.98 (s, 1H), 7.84 (s, 1H), 7.64- 7.55 (m, 4H), 7.47-7.41 (m, 4H), 7.33 (t, J = 7.3 Hz, 1H), 4.56 (dt, J = 13.4, 6.7 Hz, 3H), 4.44 (d, J = 12.3 Hz, 2H), 2.85 (d, J = 11.3 Hz, 1H), 2.67 (t, J = 12.0 Hz, 1H), 2.61- 2.53 (m, 2H), 2.30 (dd, J = 12.3, 10.4 Hz, 1H), 1.46 (d, J = 6.7 Hz, 6H), 0.97 (d, J = 6.2 Hz, 3H). |
| UB-027 | | |
| 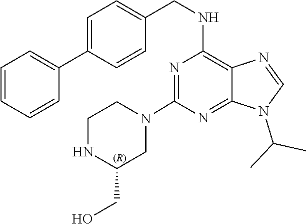 | (R)-(4-(6-(([1,1′-biphenyl]- 4-ylmethyl)amino)- 9-isopropyl-9H-purin-2- yl)piperazin-2-yl) methanol White solid compound (Yield 54%) | MS[M + 1]⁺ = 458.2; ¹H NMR (400 MHz, DMSO-d₆) δ 8.06 (s, 1H), 7.87 (s, 1H), 7.60 (dd, J = 16.3, 7.7 Hz, 4H), 7.48-7.41 (m, 4H), 7.33 (t, J = 7.3 Hz, 1H), 4.87 (s, 1H), 4.69-4.42 (m, 5H), 3.42 (d, J = 3.4 Hz, 2H), 3.01 (d, J = 11.4 Hz, 1H), 2.85 (t, J = 11.2 Hz, 1H), 2.79-2.54 (m, 3H), 1.47 (d, J = 6.7 Hz, 6H). |
| UB-028 | | |
| 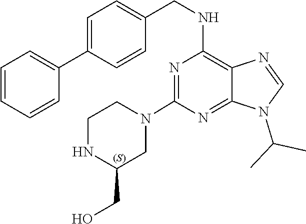 | (S)-(4-(6-(([1,1′-biphenyl]- 4-ylmethyl)amino)- 9-isopropyl-9H-purin-2- yl)piperazin-2-yl) methanol White solid compound (Yield 51.0%) | MS[M + 1]⁺ = 458.3; ¹H NMR (400 MHz, DMSO-d₆) δ 8.00 (s, 1H), 7.85 (s, 1H), 7.64- 7.60 (m, 2H), 7.58 (d, J = 8.2 Hz, 2H), 7.49- 7.41 (m, 4H), 7.33 (t, J = 7.3 Hz, 1H), 4.68 (t, J = 5.1 Hz, 1H), 4.61 (d, J = 6.3 Hz, 1H), 4.56 (dd, J = 13.4, 6.8 Hz, 2H), 4.45 (d, J = 12.6 Hz, 1H), 3.38-3.34 (m, 2H), 2.93 (d, J = 11.5 Hz, 1H), 2.76 (t, J = 12.0 Hz, 1H), 2.60 (t, J = 9.8 Hz, 2H), 2.48-2.39 (m, 1H), 1.46 (d, J = 6.7 Hz, 6H). |
| UB-029 | | |
| 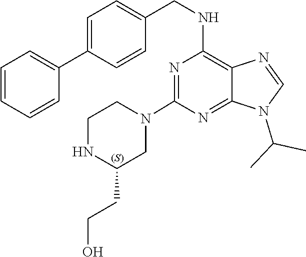 | (S)-2-(4-(6-(([1,1′-biphenyl]- 4-ylmethyl)amino)- 9-isopropyl-9H-purin- 2-yl)piperazin-2-yl) ethan-1-ol Light yellow solid compound (Yield 59.0%) | MS[M + 1]⁺ = 472.3; ¹H NMR (400 MHz, DMSO-d₆) δ 8.01 (s, 1H), 7.84 (s, 1H), 7.64 7.60 (m, 2H), 7.58 (d, J = 8.2 Hz, 2H), 7.44 (t, J = 8.1 Hz, 4H), 7.33 (t, J = 7.3 Hz, 1H), 4.57 (td, J = 13.3, 6.5 Hz, 3H), 4.48 (d, J = 10.8 Hz, 1H), 4.42 (d, J = 12.8 Hz, 1H), 3.53 (td, J = 6.4, 2.2 Hz, 2H), 2.90 (d, J = 11.8 Hz, 1H), 2.76 (d, J = 11.5 Hz, 1H), 2.62 (dd, J = 16.8, 10.5 Hz, 2H), 2.45 (d, J = 10.3 Hz, 1H), 1.54-1.48 (m, 2H), 1.46 (d, J = 6.8 Hz, 6H). |
| UB-030 | | |
| 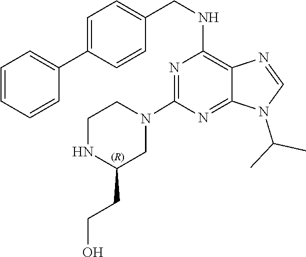 | (R)-2-(4-(6-(([1,1′-biphenyl]- 4-ylmethyl)amino)- 9-isopropyl-9H-purin- 2-yl)piperazin-2-yl) ethan-1-ol Yellow solid compound (Yield 36%) | MS[M + 1]⁺ = 472.3; ¹H NMR (400 MHz, DMSO-d₆) δ 8.19 (s, 1H), 7.91 (s, 1H), 7.64- 7.56 (m, 5H), 7.47-7.41 (m, 5H), 7.33 (dd, J = 10.4, 4.3 Hz, 2H), 4.65-4.45 (m, 6H), 3.56 (dd, J = 11.3, 5.8 Hz, 2H), 3.18 (d, J = 12.1 Hz, 1H), 3.09 (d, J = 11.0 Hz, 3H), 2.87 (s, 3H), 1.67 (td, J = 14.5, 7.0 Hz, 3H), 1.47 (d, J = 6.8 Hz, 8H). |
| UB-031 | | |
| 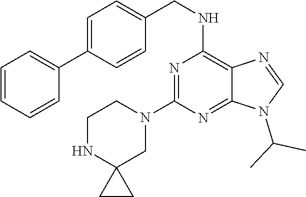 [SMILES: CC(C)n1cnc2c(NCc3ccc(-c4ccccc4)cc3)nc(N3CCNC4(CC4)C3)nc21] | N-([1,1′-biphenyl]-4-yl methyl)-9-isopropyl-2- (4,7-diazaspiro[2.5]octan- 7-yl)-9H-purin-6- amine White solid compound (Yield 48%) | MS[M + 1]⁺ = 454.3; ¹H NMR (400 MHz, DMSO-d₆) δ 8.01 (s, 1H), 7.83 (s, 1H), 7.66- 7.59 (m, 2H), 7.56 (d, J = 8.2 Hz, 2H), 7.43 (dd, J = 15.7, 7.8 Hz, 4H), 7.33 (t, J = 7.3 Hz, 1H), 4.54 (dt, J = 13.5, 6.7 Hz, 3H), 3.73- 3.63 (m, 2H), 3.53 (s, 2H), 2.74 (s, 2H), 1.45 (d, J = 6.8 Hz, 6H), 0.37 (s, 4H). |
| UB-059 | | |
| 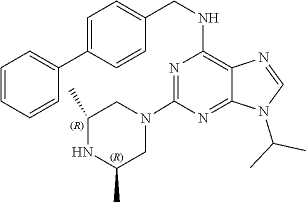 | N-([1,1′-biphenyl]-4-yl methyl)-2-((3R,5R)-3,5- dimethylpiperazin-1-yl)- 9-isopropyl-9H-purin- 6-amine White solid compound (Yield 42%) | MS[M + 1]⁺ = 456.3; ¹H NMR (400 MHz, DMSO-d₆) δ 8.03 (s, 1H), 7.84 (s, 1H), 7.65 7.60 (m, 2H), 7.57 (d, J = 8.2 Hz, 2H), 7.48 7.40 (m, 4H), 7.34 (d, J = 7.3 Hz, 1H), 4.56 (dt, J = 13.4, 6.7 Hz, 3H), 3.76 (dd, J = 12.7, 3.0 Hz, 2H), 3.41 (dd, J = 12.8, 6.2 Hz, 3H), 3.15 (s, 2H), 1.46 (dd, J = 6.7, 4.7 Hz, 6H), 1.01 (d, J = 6.3 Hz, 6H). |
| UB-032 | | |
| 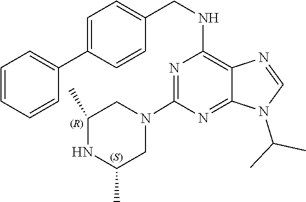 | N-([1,1′-biphenyl]-4-yl methyl)-2-((3R,5S)-3,5- dimethylpiperazin-1-yl)- 9-isopropyl-9H-purin- 6-amine White solid compound (Yield 56%) | MS[M + 1]⁺ = 456.3; ¹H NMR (400 MHz, DMSO-d₆) δ 8.01 (s, 1H), 7.84 (s, 1H), 7.66- 7.59 (m, 2H), 7.57 (d, J = 8.2 Hz, 2H), 7.49 7.38 (m, 4H), 7.33 (t, J = 7.3 Hz, 1H), 4.58 (dd, J = 13.5, 6.8 Hz, 3H), 4.50 (t, J = 11.3 Hz, 2H), 2.63 (s, 2H), 2.22 (t, J = 11.5 Hz, 2H), 1.46 (d, J = 6.8 Hz, 6H), 0.98 (d, J = 6.2 Hz, 6H). |
| UB-060 | | |
| 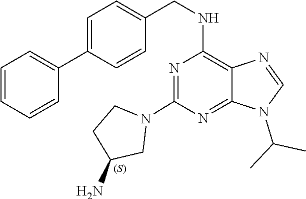 | (S)-N-([1,1′-biphenyl]- 4-ylmethyl)-2-(3-amino pyrrolidin-1-yl)-9- isopropyl- 9H-purin-6-amine White solid compound (Yield 29%) | MS[M + 1]⁺ = 428.3; 1H NMR (400 MHz, DMSO-d₆) δ 7.93 (s, 1H), 7.80 (s, 1H), 7.66- 7.55 (m, 4H), 7.51-7.40 (m, 4H), 7.33 (t, J = 7.3 Hz, 1H), 4.65 (s, 1H), 4.56 (dt, J = 13.4, 6.7 Hz, 1H), 3.66 (t, J = 5.4 Hz, 1H), 3.59 (dd, J = 11.5, 5.7 Hz, 2H), 3.53-3.44 (m, 2H), 3.27-3.18 (m, 2H), 2.06 (td, J = 13.1, 6.8 Hz, 1H), 1.90 (s, 2H), 1.73 (dd, J = 12.4, 6.4 Hz, 1H), 1.47 (d, J = 6.8 Hz, 6H). |
| UB-035 | | |
| 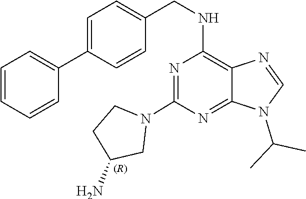 | (R)-N-([1,1′-biphenyl]- 4-ylmethyl)-2-(3-amino pyrrolidin-1-yl)-9- isopropyl- 9H-purin-6-amine White solid compound (Yield 10%) | MS[M + 1]⁺ = 428.2;. ¹H NMR (400 MHz, DMSO-d₆) δ 7.88 (s, 1H), 7.79 (d, J = 5.9 Hz, 1H), 7.64-7.56 (m, 4H), 7.45 (dd, J = 14.6, 7.5 Hz, 4H), 7.33 (t, J = 7.3 Hz, 1H), 4.78- 4.50 (m, 3H), 3.53 (dddd, J = 17.9, 15.8, 14.0, 10.9 Hz, 4H), 3.13 (dd, J = 10.8, 5.0 Hz, 1H), 1.99 (dt, J = 12.3, 6.7 Hz, 1H), 1.63 (td, J = 13.1, 6.6 Hz, 1H), 1.47 (d, J = 6.8 Hz, 6H). |
| UB-036 | | |
| 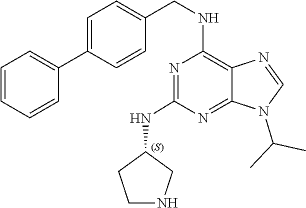 | (S)-N6-([1,1′-biphenyl]- 4-ylmethyl)-9-isopropyl- N2-(pyrrolidin-3-yl)- 9H-purin-2,6-diamine Yellow solid compound (Yield 56.3%) | MS[M + 1]⁺ = 428.2; ¹H NMR (400 MHz, DMSO-d₆) δ 7.79 (s, 1H), 7.60 (dd, J = 15.0, 7.9 Hz, 4H), 7.44 (t, J = 7.6 Hz, 4H), 7.33 (t, J = 7.3 Hz, 1H), 6.28 (d, J = 6.6 Hz, 1H), 4.67 (s, 1H), 4.53 (dt, J = 13.6, 6.7 Hz, 1H), 4.20 (d, J = 6.2 Hz, 1H), 2.90 (dd, J = 11.4, 6.6 Hz, 1H), 2.87-2.81 (m, 1H), 2.73-2.64 (m, 1H), 2.57 (dd, J = 11.0, 4.2 Hz, 1H), 1.91 (dd, J = 12.8, 6.3 Hz, 1H), 1.63-1.50 (m, 1H), 1.47 (d, J = 6.7 Hz, 6H). |
| UB-037 | | |
| 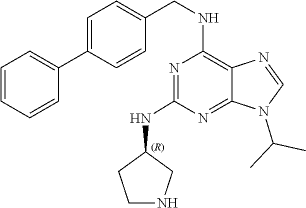 | (R)-N6-([1,1′-biphenyl]- 4-ylmethyl)-9-isopropyl- N2-(pyrrolidin-3-yl)- 9H-purin-2,6-diamine White solid compound (Yield 87%) | MS[M + 1]⁺ = 428.3; ¹H NMR (400 MHz, DMSO-d₆) δ 8.71 (s, 1H), 8.07 (s, 1H), 7.91 (s, 1H), 7.69-7.55 (m, 2H), 7.48-7.39 (m, 2H), 7.34 (t, J = 7.3 Hz, 1H), 6.73 (s, 1H), 4.67 (s, 1H), 4.56 (dt, J = 13.5, 6.8 Hz, 1H), 4.39 (d, J = 4.8 Hz, 1H), 3.40-3.27 (m, 2H), 3.28-3.16 (m, 1H), 3.12 (s, 1H), 2.12 (dd, J = 13.4, 7.0 Hz, 1H), 1.97 (d, J = 5.7 Hz, 1H), 1.48 (d, J = 6.8 Hz, 6H). |
| UB-038 | | |
| 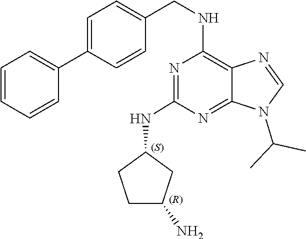 | N6-([1,1′-biphenyl]-4-yl- methyl)-N2-((1S,3R)-3- aminocyclopentyl)-9- isopropyl-9H-purin-2,6- diamine White solid compound (Yield 8%) | MS[M + 1]⁺ = 442.3; ¹H NMR (400 MHz, DMSO-d₆) 1H NMR (401 MHz, ) δ 7.88 (s, 1H), 7.82 (s, 1H), 7.61 (dd, J = 13.6, 7.8 Hz, 4H), 7.44 (dd, J = 11.9, 5.3 Hz, 4H), 7.34 (t, J = 7.3 Hz, 1H), 6.44 (d, J = 7.1 Hz, 1H), 4.66 (s, 2H), 4.53 (dt, J = 13.4, 6.8 Hz, 1H), 4.18 (d, J = 7.0 Hz, 1H), 2.43-2.30 (m, 1H), 2.00 (dd, J = 14.8, 7.0 Hz, 1H), 1.89 (s, 2H), 1.65 (d, J = 4.8 Hz, 2H), 1.47 (d, J = 6.7 Hz, 6H), 1.36-1.28 (m, 2H), 1.25 (s, 2H), 1.22-1.22 (m, 1H). |
| UB-039 | | |
| 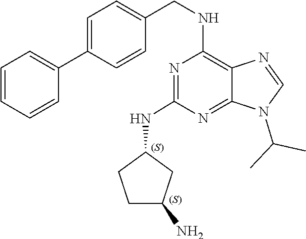 | N6-([1,1′-biphenyl]-4-yl- methyl)-N2-((1S,3S)-3- aminocyclopentyl)-9- isopropyl-9H-purin-2,6- diamine Yellow solid compound (Yield 44.8%) | MS[M + 1]⁺ = 442.3; ¹H NMR (400 MHz, DMSO-d₆) δ 7.78 (s, 1H), 7.64-7.61 (m, 2H), 7.58 (d, J = 8.2 Hz, 2H), 7.47-7.40 (m, 5H), 7.33 (t, J = 7.3 Hz, 1H), 6.19 (d, J = 7.0 Hz, 1H), 4.67 (s, 2H), 4.53 (dt, J = 13.3, 6.6 Hz, 1H), 4.31 (dd, J = 14.1, 7.1 Hz, 1H), 2.07- 2.00 (m, 1H), 1.90-1.82 (m, 1H), 1.68 (dd, J = 13.1, 6.6 Hz, 1H), 1.64-1.56 (m, 1H), 1.47 (d, J = 6.7 Hz, 6H), 1.43-1.34 (m, 2H), 1.25-1.14 (m, 2H). |
| UB-040 | | |
| 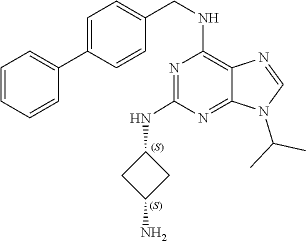 | N6-([1,1′-biphenyl]-4-yl- methyl)-N2-((1s,3s)-3- aminocyclobutyl)-9- isopropyl-9H-purin-2,6- diamine White solid compound (Yield 60%) | MS[M + 1]⁺ = 428.3; ¹H NMR (400 MHz, DMSO-d₆) δ 9.60 (d, J = 117.0 Hz, 1H), 8.73 (s, 1H), 8.31 (s, 3H), 7.64 (d, J = 7.7 Hz, 4H), 7.55-7.44 (m, 4H), 7.36 (t, J = 7.3 Hz, 1H), 5.30 (d, J = 16.8 Hz, 1H), 4.86-4.62 (m, 3H), 4.13 (s, 1H), 2.67 (s, 2H), 2.13 (d, J = 7.7 Hz, 2H), 1.53 (d, J = 6.3 Hz, 6H), 1.23 (s, 1H). |
| UB-041 | | |
| 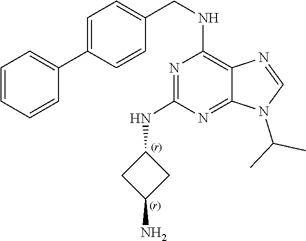 | N6-([1,1′-biphenyl]-4-yl- methyl)-N2-((1r,3r)-3- aminocyclobutyl)-9- isopropyl-9H-purin-2,6- diamine White solid compound (Yield 21%) | MS[M + 1]⁺ = 428.3; ¹H NMR (400 MHz, DMSO-d₆) δ 7.80 (d, J = 5.3 Hz, 1H), 7.66- 7.53 (m, 4H), 7.48-7.38 (m, 4H), 7.34 (dd, J = 8.3, 6.4 Hz, 1H), 6.64 (s, 1H), 4.66 (s, 1H), 4.53 (dt, J = 13.4, 6.7 Hz, 1H), 4.43 (dd, J = 13.5, 6.9 Hz, 1H), 3.67-3.47 (m, 1H), 2.27- 2.18 (m, 2H), 2.13 (s, 2H), 1.47 (d, J = 6.8 Hz, 6H). |
| UB-061 | | |

TABLE A2

| Structure & code | | |
|---|---|---|
| name | Name & properties | LCMS [M + 1]⁺ & ¹H-NMR |
| 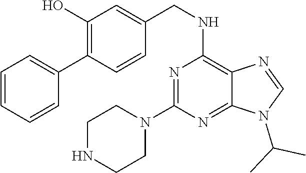 [SMILES: CC(C)n1cnc2c(NCc3ccc(-c4ccccc4)c(O)c3)nc(N3CCNCC3)nc21] | 4-((((9-isopropyl-2-(piperazin-1-yl)- 9H-purin-6-yl)amino)methyl)-[1,1′- biphenyl]-2-ol White solid compound (Yield 29%) | MS[M + 1]⁺ = 444.1; ¹H NMR (400 MHz, DMSO-d₆) δ 9.42 (s, 1H), 8.85 (s, 2H), 8.16 (d, J = 14.5 Hz, 1H), 7.93 (s, 1H), 7.56-7.44 (m, 2H), 7.36 (t, J = 7.6 Hz, 2H), 7.26 (t, J = 7.3 Hz, 1H), 7.16 (d, J = 7.8 Hz, 1H), 6.94 (s, 1H), 6.87 (d, J = 7.9 Hz, 1H), 4.73-4.32 (m, 3H), 3.87 (d, J = 4.8 Hz, 4H), 3.08 (s, 4H), 1.48 (d, J = 6.8 Hz, 6H). |
| UB-042 | | |
| 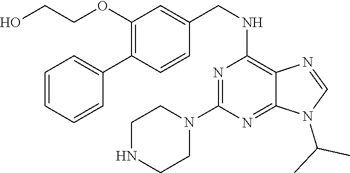 [SMILES: CC(C)n1cnc2c(NCc3ccc(-c4ccccc4)c(OCCO)c3)nc(N3CCNCC3)nc21] | 2-((4-(((9-isopropyl-2-(piperazin-1-yl)- 9H-purin-6-yl)amino)methyl)-[1,1′- biphenyl]-2-yl)oxy)ethanol Light yellow solid compound (Yield 49.0%) | MS[M + 1]⁺ = 488.3; ¹H NMR (400 MHz, DMSO-d₆) δ 7.97 (s, 1H), 7.85 (s, 1H), 7.56- 7.48 (m, 2H), 7.36 (t, J = 7.5 Hz, 2H), 7.31-7.24 (m, 1H), 7.24-7.15 (m, 2H), 7.02 (d, J = 7.7 Hz, 1H), 4.76 (s, 1H), 4.57 (dt, J = 13.5, 6.7 Hz, 3H), 3.98 (t, J = 5.2 Hz, 2H), 3.62 (dd, J = 12.8, 8.1 Hz, 6H), 2.71 (dd, J = 8.3, 3.4 Hz, 4H), 1.46 (d, J = 6.8 Hz, 6H). |
| UB-043 | | |
| 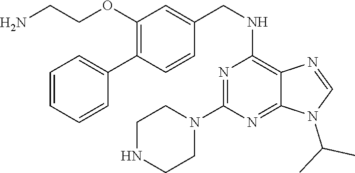 [SMILES: CC(C)n1cnc2c(NCc3ccc(-c4ccccc4)c(OCCN)c3)nc(N3CCNCC3)nc21] | N-((2-(2-aminoethoxy)-[1,l′-biphenyl]- 4-yl)methyl)-9-isopropyl-2-(piperazin- 1-yl)-9H-purin-6-amine White solid compound (Yield 78%) | MS[M + 1]⁺ = 487.3; ¹H NMR (400 MHz, DMSO-d₆) δ 7.97 (s, 1H), 7.88-7.80 (m, 1H), 7.48 (d, J = 7.2 Hz, 2H), 7.38 (t, J = 7.6 Hz, 2H), 7.28 (t, J = 7.3 Hz, 1H), 7.21 (d, J = 7.7 Hz, 1H), 7.16 (s, 1H), 7.02 (d, J = 7.7 Hz, 1H), 4.57 (dt, J = 13.5, 6.7 Hz, 3H), 3.89 (t, J = 5.7 Hz, 2H), 3.69 (s, 1H), 3.64-3.52 (m, 4H), 2.79 (t, J = 5.7 Hz, 2H), 2.75-2.63 (m, 4H), 1.46 (d, J = 6.8 Hz, 6H). |
| UB-062 | | |
| 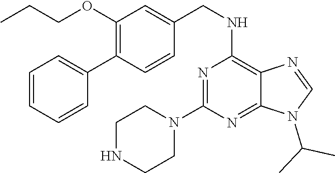 [SMILES: CCCOc1cc(CNc2nc(N3CCNCC3)nc3c2ncn3C(C)C)ccc1-c1ccccc1] | 9-isopropyl-2-(piperazin-1-yl)-N-((2- propoxy-[1,1′-biphenyl]-4-yl)methyl)- 9H-purin-6-amine White solid compound (Yield 25%) | MS[M + 1]⁺ = 486.2; ¹H NMR (400 MHz, DMSO-d₆) δ 8.24 (s, 1H), 8.04 (s, 1H), 7.87 (s, 1H), 7.46 (d, J = 7.2 Hz, 2H), 7.37 (t, J = 7.6 Hz, 2H), 7.28 (t, J = 7.3 Hz, 1H), 7.20 (d, J = 7.7 Hz, 1H), 7.15 (s, 1H), 7.01 (d, J = 7.0 Hz, 1H), 4.57 (dd, J = 13.5, 6.8 Hz, 3H), 3.89 (t, J = 6.4 Hz, 2H), 3.70 (s, 5H), 2.83 (s, 4H), 1.64 (dd, J = 13.9, 6.7 Hz, 2H), 1.47 (d, J = 6.8 Hz, 6H), 0.89 (t, J = 7.4 Hz, 3H). |
| UB-044 | | |
| 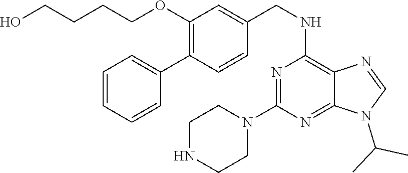 [SMILES: CC(C)n1cnc2c(NCc3ccc(-c4ccccc4)c(OCCCCO)c3)nc(N3CCNCC3)nc21] | 4-((4-(((9-isopropyl-2-(piperazin-1-yl)- 9H-purin-6-yl)amino)methyl)-[1,1′- biphenyl]-2-yl)oxy)butan-1-ol Light yellow solid compound (Yield 48.1%) | MS[M + 1]⁺ = 516.3; ¹H NMR (400 MHz, DMSO-d₆) δ 8.14 (s, 1H), 7.91 (s, 1H), 7.48- 7.42 (m, 2H), 7.36 (dd, J = 15.2, 7.4 Hz, 2H), 7.28 (t, J = 7.3 Hz, 1H), 7.19 (dd, J = 14.0, 7.2 Hz, 2H), 7.01 (d, J = 7.0 Hz, 1H), 4.58 (dd, J = 13.5, 6.7 Hz, 3H), 3.94 (t, J = 6.5 Hz, 2H), 3.82 (d, J = 3.8 Hz, 4H), 3.39 (d, J = 6.4 Hz, 2H), 3.00 (s, 4H), 1.66 (dd, J = 14.6, 6.7 Hz, 2H), 1.48 (t, J = 6.8 Hz, 8H). |
| UB-045 | | |
| 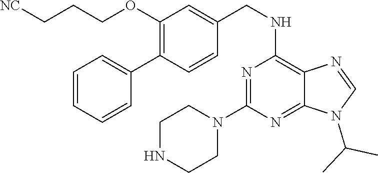 [SMILES: CC(C)n1cnc2c(NCc3ccc(-c4ccccc4)c(OCCCC#N)c3)nc(N3CCNCC3)nc21] | 4-((4-(((9-isopropyl-2-(piperazin-1-yl)- 9H-purin-6-yl)amino)methyl)-[1,1′- biphenyl]-2-yl)oxy)butyronitrile White solid compound (Yield 30%) | MS[M + 1]⁺ = 511.3; ¹H NMR (400 MHz, DMSO-d₆) δ 8.14 (s, 1H), 7.90 (s, 1H), 7.49- 7.43 (m, 2H), 7.39 (t, J = 7.6 Hz, 2H), 7.31 (dt, J = 9.3, 4.3 Hz, 1H), 7.25-7.16 (m, 2H), 7.04 (d, J = 7.8 Hz, 1H), 4.58 (dt, J = 13.5, 6.7 Hz, 2H), 4.00 (t, J = 6.0 Hz, 2H), 3.87-3.71 (m, 4H), 3.07-2.90 (m, 4H), 2.07- 1.82 (m, 2H), 1.47 (d, J = 6.8 Hz, 6H). |
| UB-063 | | |
| 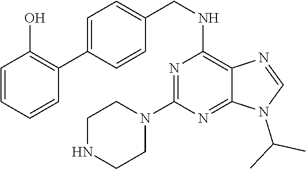 [SMILES: CC(C)n1cnc2c(NCc3ccc(-c4ccccc4O)cc3)nc(N3CCNCC3)nc21] | 4′-(((9-isopropyl-2-(piperazin-1-yl)- 9H-purin-6-yl)amino)methyl)-[1,1′- biphenyl]-2-ol White solid compound (Yield 33.9%) | MS[M + 1]⁺ = 444.3; ¹H NMR (400 MHz, DMSO-d₆) δ 9.44 (s, 1H), 7.96 (s, 1H), 7.84 (s, 1H), 7.44 (d, J = 8.2 Hz, 2H), 7.37 (d, J = 8.1 Hz, 2H), 7.20 (dd, J = 7.6, 1.6 Hz, 1H), 7.15-7.09 (m, 1H), 6.91 (d, J = 8.0 Hz, 1H), 6.84 (t, J = 7.4 Hz, 1H), 4.69-4.47 (m, 3H), 3.61 (d, J = 5.0 Hz, 4H), 2.71 (d, J = 4.5 Hz, 4H), 1.46 (d, J = 6.7 Hz, 6H). |
| UB-046 | | |
| 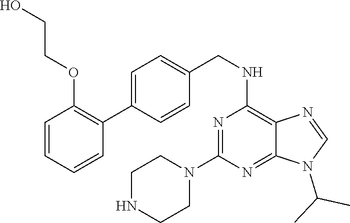 [SMILES: CC(C)n1cnc2c(NCc3ccc(-c4ccccc4OCCO)cc3)nc(N3CCNCC3)nc21] | 2-((4′-(((9-isopropyl-2-(piperazin-1-yl)- 9H-purin-6-yl)amino)methyl)- [1,1′-biphenyl]-2-yl)oxy)ethan-1-ol Light yellow solid compound (Yield 78.5%) | MS[M + 1]⁺ = 488.1; 1H NMR (400 MHz, DMSO-d₆) δ 8.04 (d, J = 19.2 Hz, 1H), 7.86 (s, 1H), 7.48 (d, J = 8.2 Hz, 2H), 7.37 (d, J = 8.2 Hz, 2H), 7.29 (dd, J = 13.3, 4.8 Hz, 2H), 7.09 (d, J = 8.0 Hz, 1H), 6.99 (t, J = 7.0 Hz, 1H), 4.58 (td, J = 13.8, 7.1 Hz, 3H), 4.01 (t, J = 5.1 Hz, 2H), 3.68-3.62 (m, 6H), 2.81- 2.72 (m, 4H), 1.47 (d, J = 6.8 Hz, 6H). |
| UB-047 | | |
| 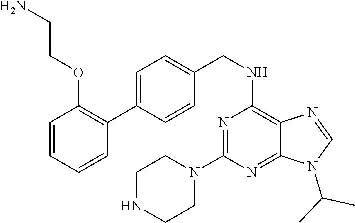 [SMILES: CC(C)n1cnc2c(NCc3ccc(-c4ccccc4OCCN)cc3)nc(N3CCNCC3)nc21] | N-((2′-(2-aminoethoxy)-[1,1′- biphenyl]-4-yl)methyl)-9-isopropyl-2- (piperazin-1-yl)-9H-purin-6-amine White solid compound (Yield 29.3%) | MS[M + 1]⁺ = 487.3; ¹H NMR (400 MHz, DMSO-d₆) δ 7.97 (s, 1H), 7.86-7.81 (s, 1H), 7.44 (d, J = 8.2 Hz, 2H), 7.38 (d, J = 8.2 Hz, 2H), 7.27 (dt, J = 7.7, 4.5 Hz, 2H), 7.08 (d, J = 7.9 Hz, 1H), 7.00 (t, J = 7.1 Hz, 1H), 4.57 (td, J = 13.6, 6.8 Hz, 3H), 3.93 (t, J = 5.6 Hz, 2H), 3.70 (d, J = 24.2 Hz, 1H), 3.65-3.55 (m, 4H), 2.81 (t, J = 5.6 Hz, 2H), 2.70 (s, 4H), 1.46 (d, J = 6.8 Hz, 6H). |
| UB-048 | | |
| 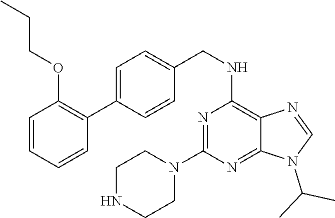 [SMILES: CCCOc1ccccc1-c1ccc(CNc2nc(N3CCNCC3)nc3c2ncn3C(C)C)cc1] | 9-isopropyl-2-(piperazin-1-yl)-N-((2′- propoxy-[1,1′-biphenyl]-4-yl)methyl)- 9H-purin-6-amine White solid compound (Yield 70%) | MS[M + 1]⁺ = 486.3; ¹H NMR (400 MHz, DMSO-d₆) δ 7.97 (s, 1H), 7.84 (s, 2H), 7.42 (d, J = 8.2 Hz, 3H), 7.37 (d, J = 8.2 Hz, 3H), 7.32-7.23 (m, 3H), 7.06 (d, J = 8.1 Hz, 1H), 6.98 (t, J = 7.3 Hz, 2H), 4.68-4.50 (m, 4H), 3.92 (t, J = 6.3 Hz, 3H), 3.65-3.54 (m, 6H), 2.70 (s, 6H), 1.65 (dd, J = 13.8, 6.6 Hz, 3H), 1.46 (d, J = 6.8 Hz, 9H), 0.91 (t, J = 7.4 Hz, 5H). |
| UB-049 | | |
| 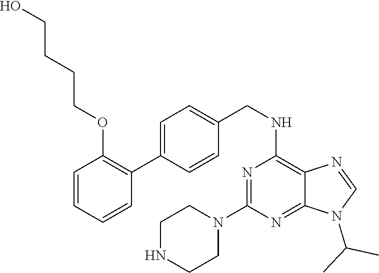 [SMILES: CC(C)n1cnc2c(NCc3ccc(-c4ccccc4OCCCCO)cc3)nc(N3CCNCC3)nc21] | 4-((4′-(((9-isopropyl-2-(piperazin-1-yl)- 9H-purin-6-yl)amino)methyl)-[1,1′- biphenyl]-2-yl)oxy)butan-1-ol White solid compound (Yield 16.7%) | MS[M + 1]⁺ = 516.3; ¹H NMR (400 MHz, DMSO-d₆) δ 7.97 (s, 1H), 7.84 (s, 1H), 7.42 (d, J = 8.3 Hz, 2H), 7.37 (d, J = 8.3 Hz, 2H), 7.31-7.22 (m, 2H), 7.07 (d, J = 7.8 Hz, 1H), 7.02-6.95 (m, 1H), 4.73-4.50 (m, 3H), 3.97 (t, J = 6.6 Hz, 2H), 3.65-3.57 (m, 4H), 3.40 (t, J = 6.4 Hz, 2H), 2.77-2.66 (m, 4H), 1.68 (s, 2H), 1.48 (dd, J = 14.1, 7.5 Hz, 8H). |
| UB-050 | | |
| 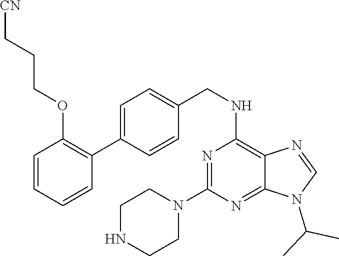 [SMILES: CC(C)n1cnc2c(NCc3ccc(-c4ccccc4OCCCC#N)cc3)nc(N3CCNCC3)nc21] | 4-((4′-(((9-isopropyl-2-(piperazin-1- yl)-9H-purin-6-yl)amino)methyl)-[1,1′- biphenyl]-2-yl)oxy)butyronitrile White solid compound (Yield 14%) | MS[M + 1]⁺ = 511.2; ¹H NMR (400 MHz, DMSO-d₆) δ 7.99 (s, 1H), 7.85 (s, 1H), 7.44- 7.37 (m, 4H), 7.34-7.24 (m, 2H), 7.10 (d, J = 8.2 Hz, 1H), 7.02 (t, J = 7.5 Hz, 1H), 4.78-4.43 (m, 3H), 4.03 (t, J = 6.0 Hz, 2H), 3.62 (s, 4H), 2.73 (s, 4H), 2.53 (d, J = 5.2 Hz, 2H), 1.95 (p, J = 6.6 Hz, 2H), 1.47 (d, J = 6.8 Hz, 6H). |
| UB-051 | | |
| 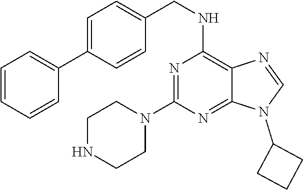 [SMILES: c1ccc(-c2ccc(CNc3nc(N4CCNCC4)nc4c3ncn4C3CCC3)cc2)cc1] | N-([1,l′-biphenyl]-4-ylmethyl)-9- cyclobutyl-2-(piperazin-1-yl)-9H- purin-6-amine White solid compound (Yield 45%) | MS[M + 1]⁺ = 440.2; ¹H NMR (400 MHz, DMSO-d₆) δ 8.01 (s, 1H), 7.94 (s, 1H), 7.66- 7.60 (m, 2H), 7.58 (d, J = 8.2 Hz, 2H), 7.49-7.39 (m, 4H), 7.33 (t, J = 7.3 Hz, 1H), 4.92-4.75 (m, 1H), 4.63 (s, 2H), 3.72-3.49 (m, 5H), 2.71 (dd, J = 14.2, 9.6 Hz, 4H), 2.59 (ddd, J = 19.3, 9.7, 2.5 Hz, 2H), 2.42-2.27 (m, 2H), 1.92-1.73 (m, 2H). |
| UB-052 | | |
| 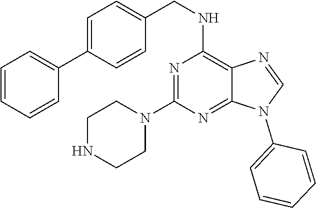 [SMILES: c1ccc(-c2ccc(CNc3nc(N4CCNCC4)nc4c3ncn4-c3ccccc3)cc2)cc1] | N-([1,1′-biphenyl]-4-ylmethyl)-9- phenyl-2-(piperazin-1-yl)-9H-purin- 6-amine Yellow solid compound (Yield 44.8%) | MS[M + 1]⁺ = 462.2; ¹H NMR (400 MHz, DMSO-d₆) δ 9.07 (s, 2H), 8.50 (s, 1H), 8.43 (s, 1H), 7.88 (d, J = 7.8 Hz, 2H), 7.65-7.60 (m, 4H), 7.57 (t, J = 7.9 Hz, 2H), 7.50-7.39 (m, 5H), 7.35 (t, J = 7.3 Hz, 1H), 4.69 (s, 2H), 3.89 (d, J = 4.7 Hz, 4H), 3.08 (s, 4H). |
| UB-053 | | |
| 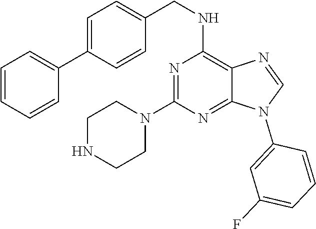 [SMILES: Fc1cccc(-n2cnc3c(NCc4ccc(-c5ccccc5)cc4)nc(N4CCNCC4)nc32)c1] | N-([1,1′-biphenyl]-4-ylmethyl)-9- (3-fluorophenyl)-2-(piperazin-1-yl)- 9H-purin-6-amine White solid compound (Yield 20%) | MS[M + 1]⁺ = 480.2; ¹H NMR (400 MHz, DMSO-d₆) δ 8.34 (s, 1H), 8.25 (s, 1H), 7.92 (dt, J = 10.8, 2.2 Hz, 1H), 7.88-7.83 (m, 1H), 7.65- 7.56 (m, 5H), 7.48-7.41 (m, 4H), 7.34 (t, J = 7.3 Hz, 1H), 7.22 (td, J = 8.5, 2.2 Hz, 1H), 4.67 (s, 2H), 3.70- 3.57 (m, 4H), 2.81-2.66 (m, 4H), 1.29 (d, J = 49.5 Hz, 3H). |
| UB-054 | | |
| 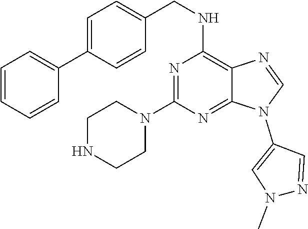 [SMILES: Cn1cc(-n2cnc3c(NCc4ccc(-c5ccccc5)cc4)nc(N4CCNCC4)nc32)cn1] | N-([1,1′-biphenyl]-4-ylmethyl)-9-(1- methyl-1H-pyrazol-4-yl)-2-(piperazin- 1-yl)-9H-purin-6-amine White solid compound (Yield 59%) | MS[M + 1]⁺ = 466.2; ¹H NMR (400 MHz, DMSO-d₆) δ 8.29 (s, 1H), 8.29 (s, 1H), 8.28-8.12 (m, 2H), 8.15 (d, J = 4.4 Hz, 1H), 7.98 (s, 1H), 7.98 (s, 1H), 7.68-7.55 (m, 4H), 7.66-7.58 (m, 4H), 7.48-7.42 (m, 4H), 7.52- 7.39 (m, 4H), 7.34 (t, J = 7.3 Hz, 1H), 7.34 (t, J = 7.3 Hz, 1H), 4.66 (s, 2H), 4.66 (s, 2H), 3.92 (s, 3H), 3.92 (s, 3H), 3.69-3.61 (m, 4H), 3.75-3.58 (m, 4H), 2.74 (s, 4H), 2.74 (s, 4H). |
| UB-055 | | |

TABLE A3

| | |
|---|---|
| UB-001 | 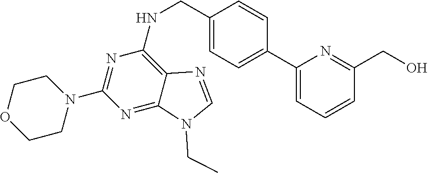 [SMILES: CCn1cnc2c(NCc3ccc(-c4cccc(CO)n4)cc3)nc(N3CCOCC3)nc21] |
| | ¹H NMR (400 MHz, DMSO-d6) δ 8.06 (s, 1H), 8.03-7.95 (m, 2H), 7.89- |
| | 7.73 (m, 3H), 7.45 (d, J = 8.1 Hz, 2H), 7.40 (d, J = 7.6 Hz, 1H), 5.43 (t, J = |
| | 5.9Hz, 1H), 4.65 (s, 1H), 4.61 (d, J = 5.8 Hz, 2H), 4.02 (q, J = 7.2 Hz, 2H), |
| | 3.61 (s, 8H), 1.35 (t, J = 7.2 Hz, 3H). LCMS [M + H]⁺ = 446.6 |
| UB-002 | 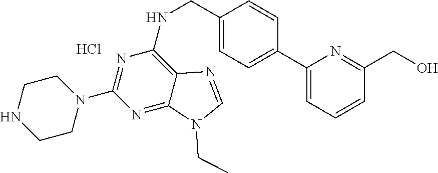 [SMILES: CCn1cnc2c(NCc3ccc(-c4cccc(CO)n4)cc3)nc(N3CCNCC3)nc21 ; Cl] |
| | ¹H NMR (400 MHz, DMSO-d6) δ 8.16 (s, 1H), 7.99 (d, J = 8.0 Hz, 2H), |
| | 7.88-7.81 (m, 2H), 7.76 (d, J = 7.8 Hz, 1H), 7.46 (d, J = 8.0 Hz, 2H), 7.41 |
| | (d, J = 7.6Hz, 1H), 5.45 (s, 1H), 4.67-4.58 (m, 3H), 4.03 (q, J = 7.2 Hz, |
| | 2H), 3.80 (t, J = 5.2 Hz, 4H), 2.96 (t, J = 5.1 Hz, 4H), 1.36 (t, J = 7.2 Hz, |
| | 3H), 1.24 (d, J = 3.5Hz, 1H) LCMS [M + H]⁺ = 445.6 |
| UB-003 | 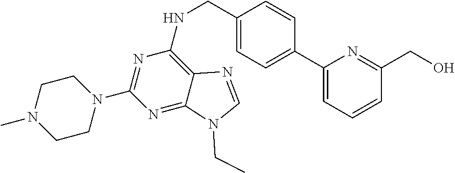 [SMILES: CCn1cnc2c(NCc3ccc(-c4cccc(CO)n4)cc3)nc(N3CCN(C)CC3)nc21] |
| | ¹H NMR (400 MHz, DMSO-d6) δ 8.05 (s, 1H), 8.01-7.96 (m, 2H), 7.87- |
| | 7.79 (m, 2H), 7.75 (d, J = 7.8 Hz, 1H), 7.45 (d, J = 8.1 Hz, 2H), 7.40 (d, J = |
| | 7.5Hz, 1H), 5.45 (t, J = 5.9 Hz, 1H), 4.62 (t, J = 8.0 Hz, 3H), 4.02 (q, J = 7.2 |
| | Hz, 2H), 3.70 (s, 4H), 2.49-2.39 (m, 4H), 2.24 (s, 3H), 1.35 (t, J = 7.2 Hz, |
| | 3H) LCMS [M + H]⁺ = 459.3 |
| UB-004 | 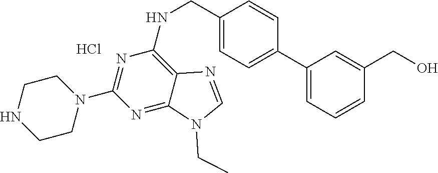 [SMILES: CCn1cnc2c(NCc3ccc(-c4cccc(CO)c4)cc3)nc(N3CCNCC3)nc21 ; Cl] |
| | ¹H NMR (400 MHz, DMSO-d6) δ 7.78 (s, 1H), 7.65-7.54 (m, 4H), 7.48- |
| | 7.40 (m, 4H), 7.37-7.30 (m, 1H), 4.63 (s, 2H), 4.01 (q, J = 7.2 Hz, 2H), |
| | 3.68-3.54 (m, 4H), 2.75-2.63 (m, 4H), 1.35 (t, J = 7.2 Hz, 3H). LCMS |
| | [M + H]⁺ = 414.4 |
| UB-005 | 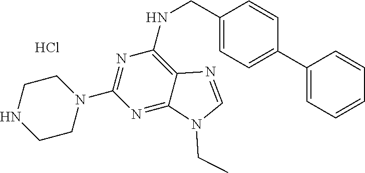 [SMILES: CCn1cnc2c(NCc3ccc(-c4ccccc4)cc3)nc(N3CCNCC3)nc21 ; Cl] |
| | ¹H NMR (400 MHz, DMSO-d₆) δ 7.99 (d, J = 8.3 Hz, 3H), 7.87-7.73 (m, |
| | 3H), 7.45 (d, J = 8.1 Hz, 2H), 7.40 (d, J = 7.6 Hz, 1H), 5.41 (s, 1H), 4.61 (d, |
| | J = 4.2 Hz, 4H), 4.01 (q, J = 7.2 Hz, 2H), 3.63 (t, J = 5.1 Hz, 4H), 2.74 (d, |
| | J = 10.2 Hz, 4H), 1.35 (t, J = 7.2 Hz, 3H). LCMS [M + H]⁺ = 444.6 |
| UB-006 | 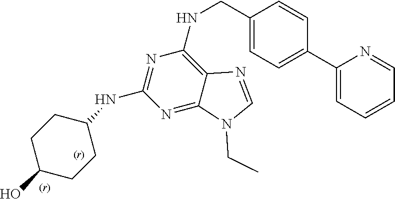 |
| | ¹H NMR (400 MHz, DMSO-d6) δ 8.67-8.60 (m, 1H), 8.04-7.97 (m, 2H), |
| | 7.93-7.82 (m, 2H), 7.72 (s, 1H), 7.45 (d, J = 8.0 Hz, 2H), 7.35-7.28 (m, |
| | 1H), 6.08 (d, J = 7.6 Hz, 1H), 4.65 (s, 2H), 4.48 (d, J = 4.5 Hz, 1H), 3.97 (q, |
| | J = 7.2 Hz, 2H), 3.61 (s, 1H), 1.79 (d, J = 11.3 Hz, 4H), 1.33 (t, J = 7.2 Hz, |
| | 3H), 1.28-1.14 (m, 5H). LCMS [M + H]⁺ = 444.9 |

TABLE A4

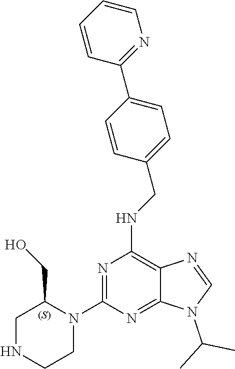

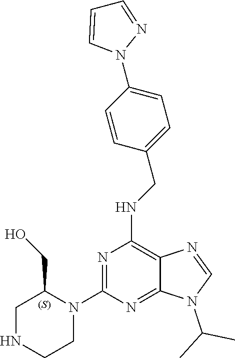

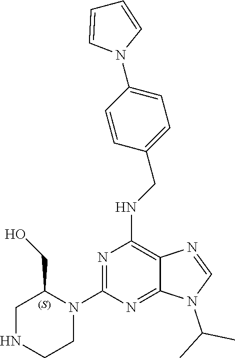

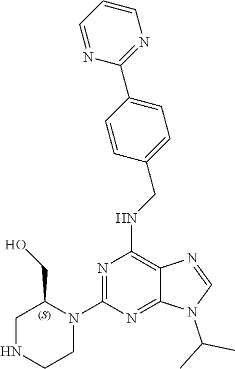

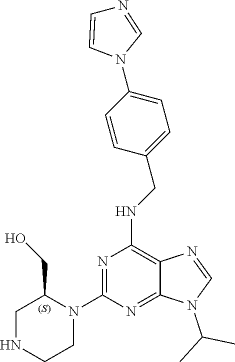

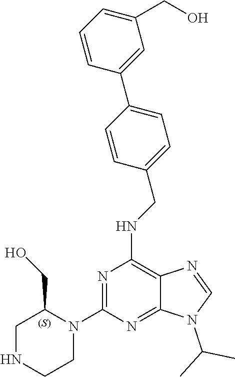

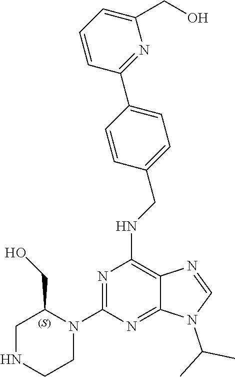

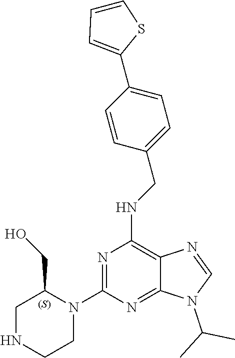

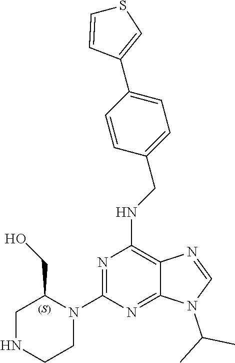

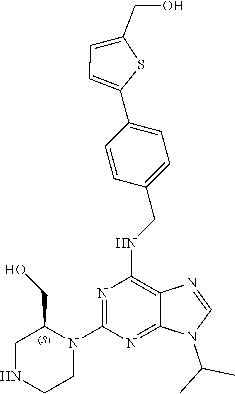

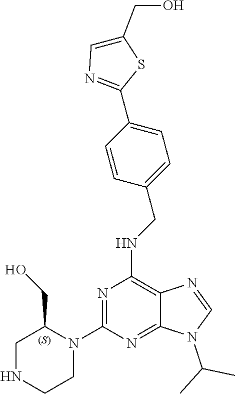

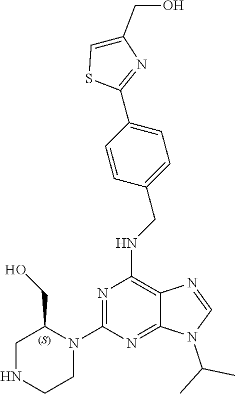

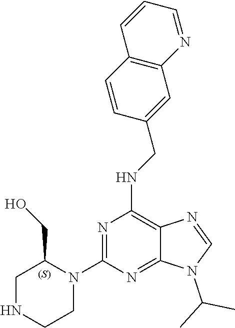

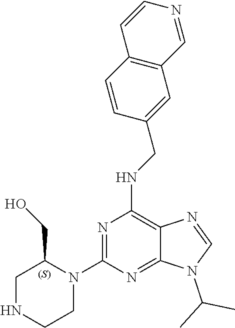

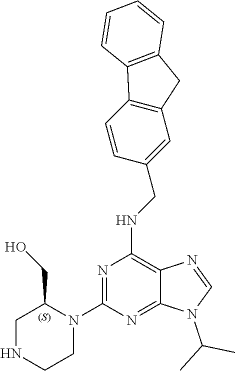

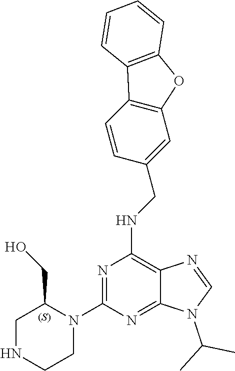

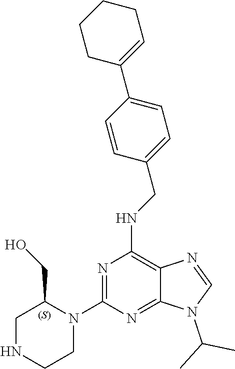

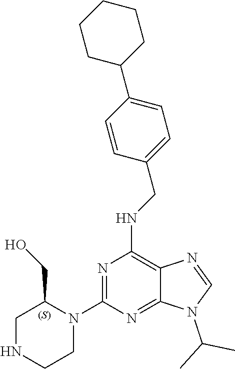

### B. Test Example

### Test Example 1: Cell Proliferation Experiment

Reagents: RPMI-1640 medium, McCoy's 5A medium, IMDM medium, MEM medium, L-15 medium, fetal bovine serum, Penicillin-Streptomycin double antibody, trypsin, etc., 2-mercaptoethanol, NEAA, pyruvate, etc.

The test cells were cultured in conventional ways, and the cells should be passed for at least 2 generations before plating.

25 μL of 1000 HT-29, N₈₇, SKOV3, Colo-205 and MDA-MB-231 cells were inoculated in a 384-well plate with black wall and transparent bottom, 25 μL of different concentrations of compounds were added, and the cells were cultured overnight under condition of 37° C. and 5% CO₂.

25 μL of 1500 H82 and H69 cells were inoculated in a 384-well plate with black wall and transparent bottom, 25 μL of different concentrations of compounds were added, and the cells were cultured for 72 hours under condition of 37° C. and 5% CO₂. The 384-well plate was equilibrated at room temperature, 25 μL of Cell Titer-Glo® reagent was added to each well, and the cells were lysed by shaking on a horizontal shaking shaker for 2 minutes, incubated at room temperature for 10 minutes to stabilize the luminescent signal, and then the chemiluminescent signal was detected using Envision.

All cells were subjected to IC₅₀ test for corresponding samples.

The fluorescence intensity of each well was detected using the Alarm blue method, and EC₅₀ was calculated.

IC₅₀ was calculated according to the following formula:

$$Y = Max + ( Min - Max ) / [ 1 + ( X / {IC}_{50} ) \times Slope ]$$

wherein, Min, Max and Slope represent the minimum, maximum and slope respectively.

The results are shown in Table 1. The compounds of the invention were subjected to cell inhibition experiments on multiple tumor cell lines. The results showed that most of the compounds exhibited strong cell killing effects in multiple tumor cells and have the potential to be developed as anti-tumor drugs.

TABLE 1

| | | Cell antiproliferation data | | | | | | |
|---|---|---|---|---|---|---|---|---|
| | | A <= 100 nM, 100 nM < B <= 300 nM, C > 300 nM | | | | | | |
| Compound | Example | | | MDA- | | | Colo- | |
| No. | No. | H82 | HT-29 | MB-231 | H69 | SKOV3 | 205 | N87 |
| UB-001 | 1 | B | A | A | A | | B | A |
| UB-002 | 2 | B | A | A | A | | A | A |
| UB-003 | 3 | | A | A | B | | A | |
| UB-004 | 4 | | A | A | C | B | B | |
| UB-005 | 5 | A | A | A | A | A | A | A |
| UB-006 | 6 | | A | A | C | A | A | |
| UB-007 | 7 | B | B | C | C | C | C | B |
| UB-008 | 8 | C | C | C | C | C | C | C |
| UB-009 | 9 | C | C | C | C | C | C | C |
| UB-010 | 10 | A | A | A | B | A | A | A |
| UB-011 | 11 | B | B | B | C | C | C | B |
| UB-012 | 12 | A | A | A | B | B | A | A |
| UB-013 | 13 | A | A | A | B | B | A | A |
| UB-014 | 14 | A | A | A | B | B | A | A |
| UB-015 | 15 | A | A | A | B | B | A | A |
| UB-017 | 17 | B | A | A | B | B | B | A |
| UB-018 | 18 | A | A | A | A | A | A | A |
| UB-019 | 19 | B | A | B | B | B | B | A |

### Test Example 2: Immunohistochemistry (IHC) Staining

Appropriate amount of compounds were added to the cultured cells. The cells were incubated in a tissue culture incubator at 37° C. for 2 hours.

After washing the cultured cells twice with PBS, 100 μL 10% neutral buffered formalin were added and the cells were fixed for 20 minutes.

After washing 3 times with PBS, immunohistochemical staining was performed using CCNK antibody and general two-step detection kit (mouse/rabbit enhanced polymer detection system, PV-9000, Zsbio Inc, Beijing).

Specifically, 100 μL of endogenous peroxidase blocker in the kit was added, cells were incubated at room temperature for 10 minutes; rinsed with PBS buffer for 3 minutes×3 times.

100 μL CCNK antibody 1:5000 dilution was added, and the cells were incubated at 37° C. for 60 minutes; rinsed with PBS buffer for 3 minutes×3 times.

100 μL reaction enhancer was added, and the cells were incubated at 37° C. for 20 minutes; rinsed with PBS buffer for 3 minutes×3 times.

100 μL enhanced enzyme-labeled goat anti-mouse/rabbit IgG polymer was added, and the cells were incubated at 37° C. for 20 minutes; rinsed with PBS buffer for 3 minutes×3 times.

The cells were placed in 75% ethanol, soaked for 3 minutes×2 times; placed in 95% ethanol, soaked for 3 minutes×2 times; after removing excess liquid, placed in anhydrous ethanol, soaked for 3 minutes×3 times; after removing excess liquid, air dried, observed under a microscope and taken pictures after sealing.

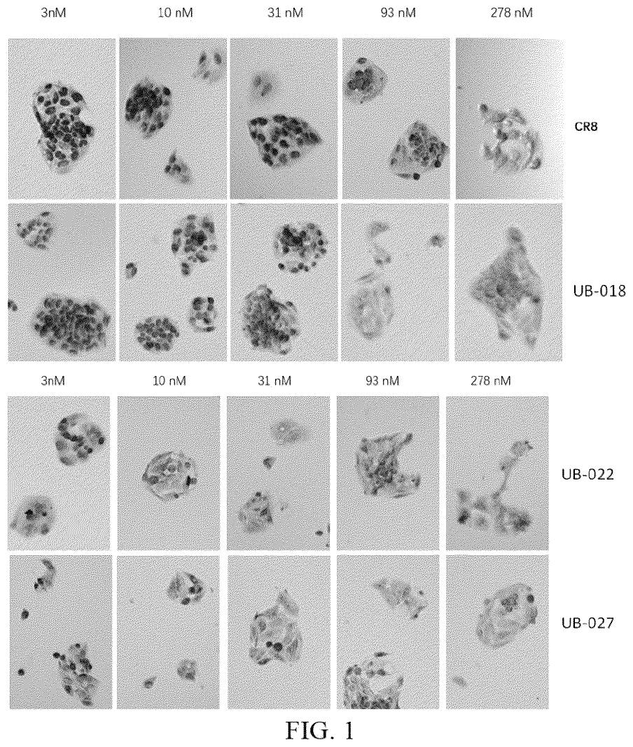

The results are shown in FIG. 1 and Table 2, it can be seen that the compounds of the invention can rapidly and effectively degrade Cyclin K proteins, and most of the compounds have significantly better activity in degrading Cyclin K than CR8.

### Test Example 3: Western Blot

A transparent 12-well plate was coated with 500 uL 0.01 mg/mL poly-D-lysine hydrobromide, and placed at 37° C. for 1 hour. The coating solution was removed, and the plate was washed twice with 1 mL PBS. 400 thousand HEK293 cells were seeded in the 12-well plate. Cells were treated with the compound for 24h, and then the culture medium was removed. After washing with PBS, the cells were lysed by adding RIPA buffer; the cell lysate were added to the loading buffer and then appropriate volume were taken and slowly added to the corresponding wells of the gel plate, and run the SDS-PAGE gel (4%-12%). After running the gel, transferred to the PVDF membrane and sealed with 5% skimmed milk powder at room temperature for 1 hour. The membrane was placed in anti-Cyclin K primary antibody diluted with 5% skimmed milk powder and shaked slowly overnight at 4 degrees. After incubation with primary antibodies, the membrane was washed 3 times using a TBST shaker; added with anti-rabbit HRP secondary antibodies diluted with 5% skimmed milk powder corresponding to the primary antibodies, and shaked slowly at room temperature for 1 hour. After incubation with secondary antibodies, the membrane was washed 3 times using the TBST shaker again. The PVDF membrane was flatted in the cassette; the strip was evenly infiltrated with ECL developer solution, and placed in ChemDoc XRS+ gel imager for taking pictures. The intensity of protein bands was analyzed quantitatively using ImageJ software.

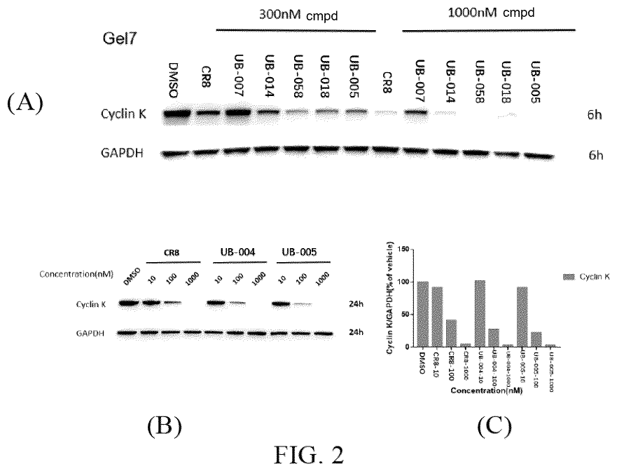

The results are shown in FIG. 2 and Table 2, it can be seen that the compounds of the invention can rapidly and effectively degrade Cyclin K proteins, and most of the compounds have significantly better activity in degrading Cyclin K than CR8.

TABLE 2

| | Cyclin K IHC data | Antiproliferative effect |
|---|---|---|
| | of HepG2 cells | on HepG2 cells |
| | A <= 31 nM, | A < 50 nM, |
| | 31 nM < B <= 93 nM, | 50 nM < B <= 100 nM, |
| | 93 nM < C <= 278 nM, | 100 nM < C <= 300 nM, |
| | D > 278 nM | D > 300 nM |
| UB-018 | B | A |
| UB-042 | B | A |
| UB-049 | D | C |
| UB-044 | D | D |
| UB-062 | D | D |
| UB-046 | D | C |
| UB-047 | D | B |
| UB-043 | D | C |
| UB-026 | B | A |
| UB-027 | A | A |
| UB-048 | D | D |
| UB-050 | D | C |
| UB-045 | D | D |
| UB-035 | D | A |
| UB-036 | C | A |
| UB-020 | B | A |
| UB-037 | C | A |
| UB-038 | C | A |
| UB-022 | A | A |
| UB-033 | C | B |
| UB-034 | C | B |
| UB-023 | C | B |
| UB-021 | C | B |
| UB-051 | D | C |
| UB-053 | D | D |
| UB-040 | B | A |
| UB-041 | C | A |
| UB-055 | D | D |
| UB-039 | C | B |
| UB-061 | B | A |
| UB-063 | D | C |
| UB-052 | C | A |
| UB-028 | A | |
| UB-025 | A | |
| UB-032 | D | |
| UB-060 | A | |
| UB-059 | B | |
| UB-054 | D | |
| UB-024 | A | |
| UB-016 | C | |
| UB-029 | B | |
| UB-030 | B | |
| UB-031 | B | |

All documents mentioned in the present invention are cited as references in this application, just as each document is individually cited as a reference. In addition, it should be understood that, after reading the above teaching content of the present invention, those skilled in the art can make various changes or modifications to the present invention, and these equivalent forms also fall within the scope defined by the appended claims of the present application.

1. A compound of formula (I), or a pharmaceutically acceptable salt thereof, 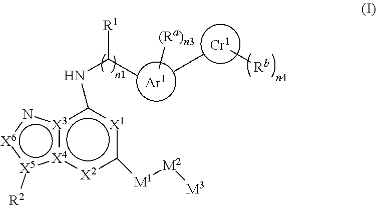
wherein,
R¹ is each independently H or C₁₋₄alkyl;
subscript n1 is 1, 2 or 3;
Ring Ar¹ is selected from the group consisting of: C₆₋₁₀ aromatic ring, 5 to 10 membered heteroaromatic ring, and 5 to 10 membered bridged ring;
Ring Cr¹ is selected from the group consisting of: H, C₃₋₁₀ carbocyclyl, 3 to 10 membered heterocyclyl, C₆₋₁₀ aryl, and 5 to 10 membered heteroaryl;
R^{a} and R^{b} are each independently selected from the group consisting of: H, R^{e} and R; or R^{a} and R^{b} together with Ring Ar¹ and Ring Cr¹ form 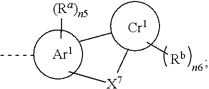
wherein,
X⁷ is each independently selected from the group consisting of: —O—, —S—, —N(R^{c})—, —C(R^{c})₂—, and —C(R^{c})₂—C(R^{c})₂—;
subscripts n5 and n6 are each independently 0, 1, 2 or 3;
R^{e} is each independently selected from the group consisting of: hydroxyl, C₁₋₆alkyl, —O—C₁₋₆alkyl, and —O—C₁₋₆alkylene-R^{f};
wherein, R^{f} is selected from the group consisting of: —CN, —OH, —NH₂, —NH(C₁₋₆alkyl), and —N(C₁₋₆alkyl)₂;
subscripts n3 and n4 are each independently 0, 1, 2, 3 or 4;
R² is selected from the group consisting of: H, CN, optionally substituted C₁₋₆alkyl, optionally substituted C₂₋₆ alkenyl, optionally substituted C₂₋₆ alkynyl, optionally substituted C₃₋₈ cycloalkyl, and optionally substituted 3 to 8 membered heterocyclyl;
X¹, X² and X⁶ are each independently N or C(R^{c});
X³, X⁴ and X⁵ are each independently N or C;
M¹ is selected from the group consisting of: null, X⁸, and (M⁴)ₛ; wherein,
X⁸ is N(R^{c}) or C(R^{c})₂;
M⁴ is each independently selected from the group consisting of: O, S, C(O)O, C(O), N(R^{c}) and C₁₋₄ alkylene;
s is 1, 2 or 3;
M² is null or a ring of formula A; 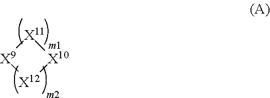
in formula A, X⁹ is the position attached to M¹, X¹⁰ is the position attached to M³; X⁹ is N or C(R^{m}), X¹⁰ is selected from the group consisting of: O, S, N or C(R^{m}); X¹¹ and X¹² are each independently selected from the group consisting of: —C(R^{m})₂—, and —N(R^{m})—;
subscripts m1 and m2 are each independently 0, 1, 2 or 3, and m1+m2≥2;
wherein, R^{m} is each independently R^{e} or R^{m1}; wherein,
R^{m1} is each independently selected from the group consisting of: hydroxyl, optionally substituted C₁₋₆ alkyl, optionally substituted C₁₋₆hydroxyalkyl, optionally substituted C₁₋₆ haloalkyl; or, two R^{m1} together form a single bond, optionally substituted C₁₋₄alkylene or optionally substituted 1 to 4 membered heteroalkylene;
M³ is selected from the group consisting of: null, R³, and —NH—R³; wherein,
R³ is selected from the group consisting of: H, optionally substituted C₁₋₆alkyl, optionally substituted C₁₋₆-hydroxyalkyl, and optionally substituted C₁₋₆-haloalkyl;
R^{c} is each independently H or C₁₋₄alkyl;
unless specifically defined, said optionally substituted means unsubstituted or one or more (such 1, 2, or 4) hydrogen atoms in the group are substituted with a substituent R, and R is selected from the group consisting of: D, halogen, C₁₋₆alkyl, C₁₋₆haloalkyl, C₁₋₆hydroxyalkyl, C₂₋₆alkenyl, C₂₋₆ alkynyl, —CN, —OR′, —NO₂, —NR′R″, —SR′, —OC(O)R′, —C(O)R′, —CO₂R′, —CONR′, —OC(O)NR′R″, —NR″C(O)R′, —NR″—C(O)NR′R″, —NR″C(O)₂R′, —S(O)R′, —S(O)₂R′, —S(O)₂NR′R″, —NR″S(O)₂R′, C₃₋₁₀cycloalkyl optionally substituted with one or more R′″, 4 to 10 membered heterocycloalkyl optionally substituted with one or more R′″, C₆₋₁₀aryl optionally substituted with one or more R′″, 5 to 10 membered heteroaryl optionally substituted with one or more R′″, —C₁₋₄alkylene-C₃₋₁₀cycloalkyl optionally substituted with one or more R′″, —C₁₋₄alkylene-4 to 10 membered heterocycloalkyl optionally substituted with one or more R′″, —C₁₋₄alkylene-C₆₋₁₀aryl optionally substituted with one or more R′″, and —C₁₋₄alkylene-5 to 10 membered heteroaryl optionally substituted with one or more R′″;
each R′ is each independently H, D, a group selected from the following group that is optionally substituted with one or more R′″: C₁₋₆alkyl, C₃₋₁₀ cycloalkyl, 4 to 10 membered heterocycloalkyl, C₆₋₁₀aryl, 5 to 10 membered heteroaryl, —C₁₋₄alkylene-C₃₋₁₀cycloalkyl, —C₁₋₄alkylene-4 to 10 membered heterocycloalkyl, —C₁₋₄alkylene-C₆₋₁₀aryl, and —C₁₋₄alkylene-5 to 10 membered heteroaryl;
each R″ is selected from the group consisting of: H, D, C₁₋₄ alkyl, C₁₋₄ haloalkyl, and C₃₋₄ cycloalkyl;
each R′″ is independently selected from the group consisting of: D, halogen, hydroxyl, nitro, CN, C₁₋₆alkyl, and C₁₋₆ haloalkyl.

2. The compound of claim 1 or the pharmaceutically acceptable salt thereof, wherein,
Ring Ar¹ is selected from the group consisting of: 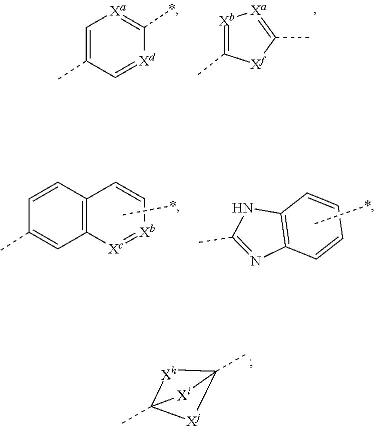
wherein, * represents the position attached to Ring Cr¹; X^{a}, X^{b}, X and X^{d} are each independently CH or N; X^{g} is selected from the group consisting of: NH, O, and S; X^{h}, Xⁱ and X^{j} are each independently —CH₂— or —CH₂—CH₂—;
and/or, Ring Cr¹ is selected from the group consisting of: 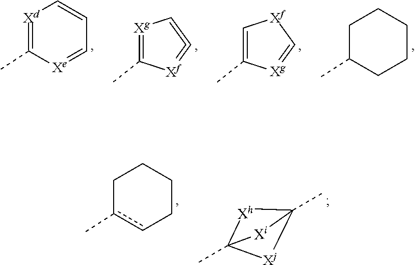
wherein, X^{d} and X^{e} are each independently N or CH; X^{f} is NH, S, or O; X^{g} is N or CH.

3. The compound of claim 1 or the pharmaceutically acceptable salt thereof, wherein, 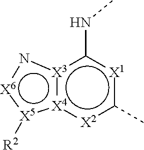
is selected from the group consisting of:

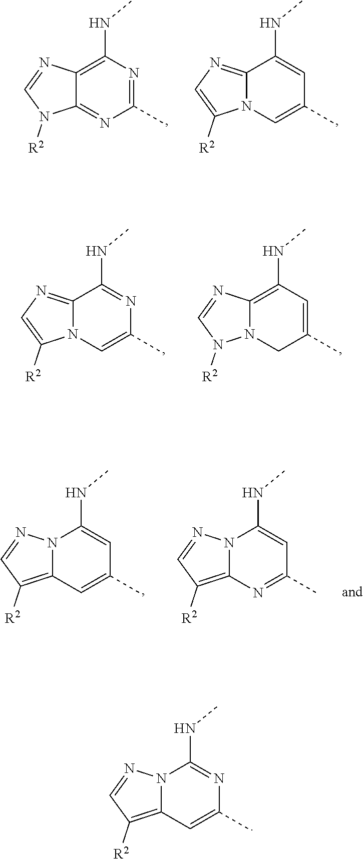

4. The compound of claim 1 or the pharmaceutically acceptable salt thereof, wherein, Ring Cr¹ is Ring Ar²; and Ring Ar² is C₆₋₁₀ Aryl.

5. The compound of claim 1 or the pharmaceutically acceptable salt thereof, wherein, Ring Ar² is phenyl.

6. The compound of claim 1 or the pharmaceutically acceptable salt thereof, wherein,
the compound is of formula I-1 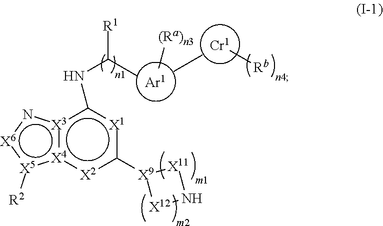
or, the compound is of formula I-2 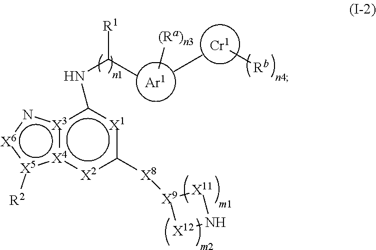
or, the compound is of formula I-3 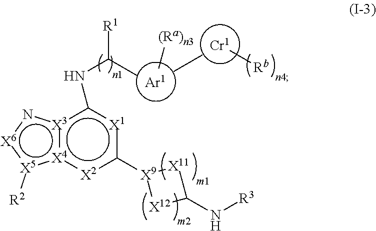
or, the compound is of formula I-4

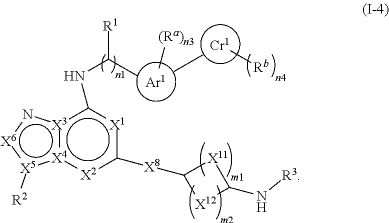

7. The compound of claim 6 or the pharmaceutically acceptable salt thereof, wherein,
the compound is a compound of formula I-1,
wherein, 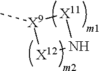
is 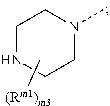
m3 is 0, 1 or 2.

8. The compound of claim 6 or the pharmaceutically acceptable salt thereof, wherein, Ring Cr¹ is Ring Ar²; and Ring Ar² is phenyl.

9. The compound of claim 1 or the pharmaceutically acceptable salt thereof, wherein the compound is selected from compounds in Table A1, A2, A3 and Table A4.

10. A pharmaceutical composition, wherein comprising
(i) the compound of claim 1 or the pharmaceutically acceptable salt thereof; and
(ii) pharmaceutically acceptable carriers.

11. A method for treating cancer, comprising administering the pharmaceutical composition of claim 10 to a subject in need thereof.

12. A conjugate or a pharmaceutically acceptable salt thereof, wherein the conjugate is a conjugate formed by the compound of claim 1 and a polypeptide element or a targeting ligand.

13. The conjugate of claim 12 or the pharmaceutically acceptable salt thereof, wherein the conjugate is of formula II, 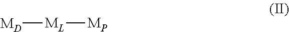
wherein,
M_{D} is a moiety derived from the compound of formula (I) according to the first aspect;
M_{L} is null or a linker moiety for connecting M_{D} and M_{P};
M_{P} is a moiety derived from the polypeptide element or the targeting ligand.

14. A pharmaceutical composition comprising
(i) the conjugate of claim 12 or the pharmaceutically acceptable salt thereof; and
(ii) pharmaceutically acceptable carriers.

15. A method for treating cancer, comprising administering the pharmaceutical composition of claim 12 to a subject in need thereof.
